SEQUENCE LISTING (67 sequences)

SEQ ID NO: 1 - protein, 863 aa, Escherichia coli
MQEQYRPEEIESKVQLHWDEKRTFEVTEDESKEKYYCLSMLPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWYYARYTCPQYKEGMLDSEAANYWLPVDIYIGGIEHAIMHLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 2 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 3 - protein, 863 aa, Artificial Sequence
  Description of Artificial Sequence Synthetic polypeptide
MEEQYRPEEIESKVQLHWDKKRTFEVTEDESKEKYYCLSVSPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDRFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWSYARYTCPQYKEGMLDSEAANYWLPVDILIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 4 - protein, 863 aa, Artificial Sequence
  Description of Artificial Sequence Synthetic polypeptide
MEEQYRPEEIESKVQLHWDEKRTFEVTEDESKEKYYCLSILPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 5 - protein, 863 aa, Artificial Sequence
  Description of Artificial Sequence Synthetic polypeptide
MEEQYRPEEIESKVQLHWDMKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 6 - protein, 863 aa, Artificial Sequence
  Description of Artificial Sequence Synthetic polypeptide
MEEQYRPEEIESKVQLHWDVKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 7 - protein, 863 aa, Artificial Sequence
  Description of Artificial Sequence Synthetic polypeptide
MEEQYRPEEIESKVQLHWDKKRTFEVTEDESKEKYYCLSIVPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 8 - protein, 863 aa, Artificial Sequence
  Description of Artificial Sequence Synthetic polypeptide
MEEQYRPEEIESKVQLHWDKKRTFEVTEDESKEKYYCLSIAPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 9 - protein, 862 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWAYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 10 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWHYARYTCPQYKEGMLDSEAANYWLPVDIAIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 11 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIIIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 12 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIVIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 13 - protein, 863 aa, Artificial Sequence
  Description of Artificial Sequence Synthetic polypeptide
MEEQYRPEEIESKVQLHWDKKRTFEVTEDESKEKYYCLSISPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWIYARYTCPQYKEGMLDSEAANYWLPVDIGIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 14 - protein, 863 aa, Artificial Sequence
  Description of Artificial Sequence Synthetic polypeptide
  MOD_RES (2)..(2): Gln or Glu
  MOD_RES (20)..(20): Glu, Lys, Val or Met
  MOD_RES (40)..(40): Met, Ile, or Val
  MOD_RES (41)..(41): Leu, Ser, Val, or Ala
  MOD_RES (252)..(252): Thr, Ala, or Arg
  MOD_RES (499)..(499): Tyr, Ala, Ile, His, or Ser
  MOD_RES (527)..(527): Tyr, Ala, Ile, Leu, or Val
  MOD_RES (537)..(537): His or Gly
MXEQYRPEEIESKVQLHWDXKRTFEVTEDESKEKYYCLSXXPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDXFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWXYARYTCPQYKEGMLDSEAANYWLPVDIXIGGIEHAIMXLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 15 - protein, 863 aa, Artificial Sequence
  Description of Artificial Sequence Synthetic polypeptide
  MOD_RES (20)..(20): Lys, Val or Met
  MOD_RES (41)..(41): Ser, Val, or Ala
  MOD_RES (499)..(499): Ala, Ile, or His
  MOD_RES (527)..(527): Ala, Ile, or Val
MEEQYRPEEIESKVQLHWDXKRTFEVTEDESKEKYYCLSIXPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWXYARYTCPQYKEGMLDSEAANYWLPVDIXIGGIEHAIMGLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGGPV

SEQ ID NO: 16 - DNA, 84 bp, Artificial Sequence
  Description of Artificial Sequence Synthetic oligonucleotide
gcccggatggtggaatcggtagacacaagggattctaaatccctcggcgttcgcgctgtgcgggttcaagtcccgctccgggta

SEQ ID NO: 17 - DNA, 84 bp, Artificial Sequence
  Description of Artificial Sequence Synthetic oligonucleotide
gcccggatggtggaatcggtagacacaagggatttcaaatccctcggcgttcgcgctgtgcgggttcaagtcccgctccgggta

SEQ ID NO: 18 - DNA, 84 bp, Artificial Sequence
  Description of Artificial Sequence Synthetic oligonucleotide
gcccggatggtggaatcggtagacacaagggattttaaatccctcggcgttcgcgctgtgcgggttcaagtcccgctccgggta

SEQ ID NO: 19 - DNA, 84 bp, Artificial Sequence
  Description of Artificial Sequence Synthetic oligonucleotide
gcccggatggtggaatcggtagacacaagggactctaaatccctcggcgttcgcgctgtgcgggttcaagtcccgctccgggca

SEQ ID NO: 20 - DNA, 84 bp, Artificial Sequence
  Description of Artificial Sequence Synthetic oligonucleotide
gcccggatggtggaatcggtagacacaagggacttcaaatccctcggcgttcgcgctgtgcgggttcaagtcccgctccgggca

SEQ ID NO: 21 - DNA, 84 bp, Artificial Sequence
  Description of Artificial Sequence Synthetic oligonucleotide
gcccggatggtggaatcggtagacacaagggactttaaatccctcggcgttcgcgctgtgcgggttcaagtcccgctccgggca

SEQ ID NO: 22 - DNA, 87 bp, Artificial Sequence
  Description of Artificial Sequence Synthetic oligonucleotide
gcccggatggtggaatcggtagacacaagggattctaaatccctcggcgttcgcgctgtgcgggttcaagtcccgctccgggtacca

SEQ ID NO: 23 - DNA, 87 bp, Artificial Sequence
  Description of Artificial Sequence Synthetic oligonucleotide
gcccggatggtggaatcggtagacacaagggatttcaaatccctcggcgttcgcgctgtgcgggttcaagtcccgctccgggtacca

SEQ ID NO: 24 - DNA, 87 bp, Artificial Sequence
  Description of Artificial Sequence Synthetic oligonucleotide
gcccggatggtggaatcggtagacacaagggattttaaatccctcggcgttcgcgctgtgcgggttcaagtcccgctccgggtacca

SEQ ID NO: 25 - DNA, 87 bp, Artificial Sequence
  Description of Artificial Sequence Synthetic oligonucleotide
gcccggatggtggaatcggtagacacaagggactctaaatccctcggcgttcgcgctgtgcgggttcaagtcccgctccgggcacca

SEQ ID NO: 26 - DNA, 87 bp, Artificial Sequence
  Description of Artificial Sequence Synthetic oligonucleotide
gcccggatggtggaatcggtagacacaagggacttcaaatccctcggcgttcgcgctgtgcgggttcaagtcccgctccgggcacca

SEQ ID NO: 27 - DNA, 87 bp, Artificial Sequence
  Description of Artificial Sequence Synthetic oligonucleotide
gcccggatggtggaatcggtagacacaagggactttaaatccctcggcgttcgcgctgtgcgggttcaagtcccgctccgggcacca

SEQ ID NO: 28 - DNA, 87 bp, Artificial Sequence
  Description of Artificial Sequence Synthetic oligonucleotide
  modified_base (35)..(37): This region may encompass “cta”, “tca”, or “tta”
gcccggatggtggaatcggtagacacaagggactnnnaatccctcggcgttcgcgctgtgcgggttcaagtcccgctccgggcacca

SEQ ID NO: 29 - DNA, 87 bp, Artificial Sequence
  Description of Artificial Sequence Synthetic oligonucleotide
  modified_base (35)..(37): This region may encompass “cta”, “tca”, or “tta”
gggcgtgtggtggaatcggtagacacaagggattnnnaatccctcggcgttcgcgctgtgcgggttcaagtcccgccgcgcccacca

SEQ ID NO: 30 - DNA, 87 bp, Artificial Sequence
  Description of Artificial Sequence Synthetic oligonucleotide
  modified_base (35)..(37): This region may encompass “cta”, “tca”, or “tta”
gggcgcgtggtggaatcggtagacacaagggattnnnaatccctcggcgttcgcgctgtgcgggttcaagtcccgccgcgcccacca

SEQ ID NO: 31 - DNA, 87 bp, Artificial Sequence
  Description of Artificial Sequence Synthetic oligonucleotide
  modified_base (35)..(37): This region may encompass “cta”, “tca”, or “tta”
gggcatgtggtggaatcggtagacacaagggattnnnaatccctcggcgttcgcgctgtgcgggttcaagtcccgccatgcccacca

SEQ ID NO: 32 - DNA, 87 bp, Artificial Sequence
  Description of Artificial Sequence Synthetic oligonucleotide
  modified_base (35)..(37): This region may encompass “cta”, “tca”, or “tta”
gggcacgtggtggaatcggtagacacaagggattnnnaatccctcggcgttcgcgctgtgcgggttcaagtcccgccgtgcccacca

SEQ ID NO: 33 - DNA, 87 bp, Artificial Sequence
  Description of Artificial Sequence Synthetic oligonucleotide
  modified_base (35)..(37): This region may encompass “cta”, “tca”, or “tta”
gggggtgtggtggaatcggtagacacaagggattnnnaatccctcggcgttcgcgctgtgcgggttcaagtcccgccgcccccacca

SEQ ID NO: 34 - DNA, 87 bp, Artificial Sequence
  Description of Artificial Sequence Synthetic oligonucleotide
  modified_base (35)..(37): This region may encompass “cta”, “tca”, or “tta”
gggggcgtggtggaatcggtagacacaagggattnnnaatccctcggcgttcgcgctgtgcgggttcaagtcccgccgtccccacca

SEQ ID NO: 35 - DNA, 87 bp, Artificial Sequence
  Description of Artificial Sequence Synthetic oligonucleotide
  modified_base (35)..(37): This region may encompass “cta”, “tca”, or “tta”
ggggatgtggtggaatcggtagacacaagggattnnnaatccctcggcgttcgcgctgtgcgggttcaagtcccgccgtccccacca

SEQ ID NO: 36 - DNA, 87 bp, Artificial Sequence
  Description of Artificial Sequence Synthetic oligonucleotide
  modified_base (35)..(37): This region may encompass “cta”, “tca”, or “tta”
ggggacgtggtggaatcggtagacacaagggattnnnaatccctcggcgttcgcgctgtgcgggttcaagtcccgccgtccccacca

SEQ ID NO: 37 - DNA, 87 bp, Artificial Sequence
  Description of Artificial Sequence Synthetic oligonucleotide
  modified_base (35)..(37): This region may encompass “cta”, “tca”, or “tta”
gcccgtatggtggaatcggtagacacaagggattnnnaatccctcggcgttcgcgctgtgcgggttcaagtcccgctgcgggcacca

SEQ ID NO: 38 - DNA, 87 bp, Artificial Sequence
  Description of Artificial Sequence Synthetic oligonucleotide
  modified_base (35)..(37): This region may encompass “cta”, “tca”, or “tta”
gggatagtggtggaatcggtagacacaagggattnnnaatccctcggcgttcgcgctgtgcgggttcaagtcccgcctatcccacca

SEQ ID NO: 39 - DNA, 87 bp, Artificial Sequence
  Description of Artificial Sequence Synthetic oligonucleotide
  modified_base (35)..(37): This region may encompass “cta”, “tca”, or “tta”
gggcatgtggtggaatcggtagacacaagggattnnnaatccctcggcgttcgcgctgtgcgggttcaagtcccgccgtgcccacca

SEQ ID NO: 40 - DNA, 87 bp, Artificial Sequence
  Description of Artificial Sequence Synthetic oligonucleotide
  modified_base (35)..(37): This region may encompass “cta”, “tca”, or “tta”
gggcagatggtggaatcggtagacacaagggattnnnaatccctcggcgttcgcgctgtgcgggttcaagtcccgctctgcccacca

SEQ ID NO: 41 - DNA, 87 bp, Artificial Sequence
  Description of Artificial Sequence Synthetic oligonucleotide
  modified_base (35)..(37): This region may encompass “cta”, “tca”, or “tta”
gggcgtatggtggaatcggtagacacaagggattnnnaatccctcggcgttcgcgctgtgcgggttcaagtcccgctgcgcccacca

SEQ ID NO: 42 - DNA, 87 bp, Artificial Sequence
  Description of Artificial Sequence Synthetic oligonucleotide
  modified_base (35)..(37): This region may encompass “cta”, “tca”, or “tta”
gcacacatggtggaatcggtagacacaagggattnnnaatccctcggcgttcgcgctgtgcgggttcaagtcccgctgtgtgcacca

SEQ ID NO: 43 - protein, 334 aa, Artificial Sequence
  Description of Artificial Sequence Synthetic polypeptide
MTKPIVFSGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLVPVGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 44 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLGPCGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 45 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLAPAGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 46 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLSPAGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 47 - protein, 334 aa, Artificial Sequence
  Description of Artificial Sequence Synthetic polypeptide
MTKPIVFAGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLGPAGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 48 - DNA, 76 bp, Artificial Sequence
  Description of Artificial Sequence Synthetic oligonucleotide
aggggcgtagttcaattggtagagcaccggtctccaaaaccgggtgttgggagttcgagtctctccgcccctgcca

SEQ ID NO: 49 - DNA, 76 bp, Artificial Sequence
  Description of Artificial Sequence Synthetic oligonucleotide
aggggcgtagttcaattggtagagcaccggtctctaaaaccgggtgttgggagttcgagtctctccgcccctgcca

SEQ ID NO: 50 - DNA, 76 bp, Artificial Sequence
  Description of Artificial Sequence Synthetic oligonucleotide
aggggcgtagttcaattggtagagcaccggtcttcaaaaccgggtgttgggagttcgagtctctccgcccctgcca

SEQ ID NO: 51 - DNA, 76 bp, Artificial Sequence
  Description of Artificial Sequence Synthetic oligonucleotide
aggggcgtagttcaattggtagagcaccggtctttaaaaccgggtgttgggagttcgagtctctccgcccctgcca

SEQ ID NO: 52 - DNA, 75 bp, Artificial Sequence
  Description of Artificial Sequence Synthetic oligonucleotide
  modified_base (34)..(36): This region may encompass “cta”, “tca”, or “tta”
tggggtatcgccaagcggtaaggcaccggattcnnnttccggcattccgaggttcgaatcctcgtaccccagcca

SEQ ID NO: 53 - DNA, 76 bp, Artificial Sequence
  Description of Artificial Sequence Synthetic oligonucleotide
  modified_base (34)..(36): This region may encompass “cta”, “tca”, or “tta”
aggggcatagctcaagcggtaaagcaccggactnnnaaaccggcagtccgaagttcgaatccccccaccccagcca

SEQ ID NO: 54 - DNA, 2589 bp, Escherichia coli
atgcaggaacagtatcgcccggaagaaattgaaagcaaagtgcagctgcattgggatgaaaaacgcacctttgaagtgaccgaagatgaaagcaaagaaaaatattattgcctgagcatgctgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatacctttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggtattatgcgcgctatacctgcccgcagtataaagaaggcatgctggatagcgaagcggcgaactattggctgccggtggatatttatattggcggcattgaacatgcgattatgcatctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 55 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 56 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcgtgagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatcgctttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggagctatgcgcgctatacctgcccgcagtataaagaaggcatgctggatagcgaagcggcgaactattggctgccggtggatattctgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 57 - DNA, 2589 bp, Artificial Sequence
  Description of Artificial Sequence Synthetic polynucleotide
atggaagaacagtatcgcccggaagaaattgaaagcaaagtgcagctgcattgggatgaaaaacgcacctttgaagtgaccgaagatgaaagcaaagaaaaatattattgcctgagcattctgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 58 - DNA, 2589 bp, Artificial Sequence
  Description of Artificial Sequence Synthetic polynucleotide
atggaagaacagtatcgcccggaagaaattgaaagcaaagtgcagctgcattgggatatgaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 59 - DNA, 2589 bp, Artificial Sequence
  Description of Artificial Sequence Synthetic polynucleotide
atggaagaacagtatcgcccggaagaaattgaaagcaaagtgcagctgcattgggatgtgaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 60 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattgtgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 61 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattgcgccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 62 - DNA, 2586 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctgggcgtatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 63 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggcattatgcgcgctatacctgcccgcagtataaagaaggcatgctggatagcgaagcggcgaactattggctgccggtggatattgcgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 64 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattattattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 65 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattgtgattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 66 - DNA, 2589 bp, Artificial Sequence
  Description of Artificial Sequence Synthetic polynucleotide
atggaagaacagtatcgcccggaagaaattgaaagcaaagtgcagctgcattgggataaaaaacgcacctttgaagtgaccgaagatgaaagcaaagaaaaatattattgcctgagcattagcccgtatccgagcggccgcctgcatatgggccatgtgcgcaactataccattggcgatgtgattgcgcgctatcagcgcatgctgggcaaaaacgtgctgcagccgattggctgggatgcgtttggcctgccggcggaaggcgcggcggtgaaaaacaacaccgcgccggcgccgtggacctatgataacattgcgtatatgaaaaaccagctgaaaatgctgggctttggctatgattggagccgcgaactggcgacctgcaccccggaatattatcgctgggaacagaaattttttaccgaactgtataaaaaaggcctggtgtataaaaaaaccagcgcggtgaactggtgcccgaacgatcagaccgtgctggcgaacgaacaggtgattgatggctgctgctggcgctgcgataccaaagtggaacgcaaagaaattccgcagtggtttattaaaattaccgcgtatgcggatgaactgctgaacgatctggataaactggatcattggccggataccgtgaaaaccatgcagcgcaactggattggccgcagcgaaggcgtggaaattacctttaacgtgaacgattatgataacaccctgaccgtgtataccacccgcccggatgcgtttatgggctgcacctatctggcggtggcggcgggccatccgctggcgcagaaagcggcggaaaacaacccggaactggcggcgtttattgatgaatgccgcaacaccaaagtggcggaagcggaaatggcgaccatggaaaaaaaaggcgtggataccggctttaaagcggtgcatccgctgaccggcgaagaaattccggtgtgggcggcgaactttgtgctgatggaatatggcaccggcgcggtgatggcggtgccgggccatgatcagcgcgattatgaatttgcgagcaaatatggcctgaacattaaaccggtgattctggcggcggatggcagcgaaccggatctgagccagcaggcgctgaccgaaaaaggcgtgctgtttaacagcggcgaatttaacggcctggatcatgaagcggcgtttaacgcgattgcggataaactgaccgcgatgggcgtgggcgaacgcaaagtgaactatcgcctgcgcgattggggcgtgagccgccagcgctattggggcgcgccgattccgatggtgaccctggaagatggcaccgtgatgccgaccccggatgatcagctgccggtgattctgccggaagatgtggtgatggatggcattaccagcccgattaaagcggatccggaatgggcgaaaaccaccgtgaacggcatgccggcgctgcgcgaaaccgatacctttgatacctttatggaaagcagctggatttatgcgcgctatacctgcccgcagtataaagaaggcatgctggatagcgaagcggcgaactattggctgccggtggatattggcattggcggcattgaacatgcgattatgggcctgctgtattttcgcttttttcataaactgatgcgcgatgcgggcatggtgaacagcgatgaaccggcgaaacagctgctgtgccagggcatggtgctggcggatgcgttttattatgtgggcgaaaacggcgaacgcaactgggtgagcccggtggatgcgattgtggaacgcgatgaaaaaggccgcattgtgaaagcgaaagatgcggcgggccatgaactggtgtataccggcatgagcaaaatgagcaaaagcaaaaacaacggcattgatccgcaggtgatggtggaacgctatggcgcggataccgtgcgcctgtttatgatgtttgcgagcccggcggatatgaccctggaatggcaggaaagcggcgtggaaggcgcgaaccgctttctgaaacgcgtgtggaaactggtgtatgaacataccgcgaaaggcgatgtggcggcgctgaacgtggatgcgctgaccgaaaaccagaaagcgctgcgccgcgatgtgcataaaaccattgcgaaagtgaccgatgatattggccgccgccagacctttaacaccgcgattgcggcgattatggaactgatgaacaaactggcgaaagcgccgaccgatggcgaacaggatcgcgcgctgatgcaggaagcgctgctggcggtggtgcgcatgctgaacccgtttaccccgcatatttgctttaccctgtggcaggaactgaaaggcgaaggcgatattgataacgcgccgtggccggtggcggatgaaaaagcgatggtggaagatagcaccctggtggtggtgcaggtgaacggcaaagtgcgcgcgaaaattaccgtgccggtggatgcgaccgaagaacaggtgcgcgaacgcgcgggccaggaacatctggtggcgaaatatctggatggcgtgaccgtgcgcaaagtgatttatgtgccgggcaaactgctgaacctggtggtgggcggcccggtg

SEQ ID NO: 67 - DNA, 87 bp, Artificial Sequence
  Description of Artificial Sequence Synthetic oligonucleotide
  modified_base (35)..(37): This region may encompass “cta”, “tca”, or “tta”
gggcaagtggtggaatcggtagacacaagggattnnnaatccctcggcgttcgcgctgtgcgggttcaagtcccgccttgcccacca

(19) United States
(12) Patent Application Publication
(10) Pub. No.: US 2022/0325269 A1
(43) Pub. Date: Oct. 13, 2022

(54) AMINOACYL-TRNA SYNTHETASES AND CELL LINES FOR SITE-SPECIFIC INCORPORATION OF UNNATURAL AMINO ACIDS

(71) Applicant: BrickBio, Inc., Woburn, MA (US)

(72) Inventor: James Sebastian Italia, Charlestown, MA (US)

(21) Appl. No.: 17/633,672

(22) PCT Filed: Aug. 7, 2020
(86) PCT No.: PCT/US2020/045506

§ 371 Date: Feb. 8, 2022

Related U.S. Application Data
(60) Provisional application No. 62/884,465, filed on Aug. 8, 2019.
(60) Provisional application No. 62/884,454, filed on Aug. 8, 2019.
(60) Provisional application No. 62/936,860, filed on Nov. 18, 2019.

(51) Int. Cl. C12N 15/10; C12P 21/02; C12N 9/00; C12N 15/67

(52) U.S. Cl. CPC C12N 15/102; C12P 21/02; C12N 9/93; C12Y 601/01004; C12N 15/67

(57) ABSTRACT

The invention relates generally to engineered tRNAs, engineered aminoacyl-tRNA synthetases, unnatural amino acids, and cells comprising the same, and their use in the incorporation of unnatural amino acids into proteins.

### CROSS-REFERENCE TO RELATED APPLICATIONS

This application claims the benefit of and priority to U.S. Provisional Patent Application Nos. 62/884,454, filed Aug. 8, 2019, 62/884,465, filed Aug. 8, 2019, and 62/936,860, filed Nov. 18, 2019, each of which are incorporated herein by a reference in their entirety for all purposes.

### FIELD OF THE INVENTION

The invention relates generally to engineered tRNAs, engineered aminoacyl-tRNA synthetases, unnatural amino acids, and cells comprising the same, and their use in the incorporation of unnatural amino acids into proteins.

### BACKGROUND

In nature, proteins are produced in cells via processes known as transcription and translation. During transcription, a gene comprising a series of codons that collectively encode a protein of interest is transcribed into messenger RNA (mRNA). During translation, a ribosome, attaches to and moves along the mRNA and incorporates specific amino acids into a polypeptide chain being synthesized (translated) from the mRNA at positions corresponding to the codons to produce the protein. During translation specific, naturally occurring amino acids coupled to transfer RNAs (tRNAs) enter the ribosome. The tRNAs, which contain an anti-codon sequence, hybridize to their respective codon sequences in mRNA and transfer the amino acid they are carrying into the nascent protein chain at the appropriate position as the protein is synthesized.

Over the :last few decades, significant efforts have been made to produce homogenous preparations of site-specifically modified proteins, e.g., mammalian proteins, on commercial scale quantities for use in a variety of applications, including, for example, therapeutics and diagnostics. Furthermore, efforts have been made to produce these modified mammalian proteins in eukaryotic cells (e.g., mammalian cells) because the proteins may be more readily produced in a properly folded and fully active form and/or post-translationally modified in a manner similar to the native protein naturally produced in a mammalian cell.

One approach for producing proteins that contain site-specific modifications involves the site-specific incorporation of one or more unnatural amino acids (UAAs) into a protein of interest. The ability to site-specifically incorporate UAAs into proteins in vivo has become a powerful tool to augment protein function or introduce new chemical functionalities not found in nature. The core elements required for this technology include: an engineered tRNA, an engineered aminoacyl-tRNA synthetase (aaRS) that charges the tRNA with a UAA, and a unique codon, e.g., a stop codon, directing the incorporation of the UAA into the protein as it is being synthesized.

Central to this approach is the use of an engineered tRNA/aaRS pair in which the aaRS charges the tRNA with the UAA of interest without cross-reacting with the tRNAs and amino acids normally present in the expression host cell. This has been accomplished by using an engineered tRNA/aaRS pair derived from an organism in different domain of life as the expression host cell so as to maximize the orthogonality between the engineered tRNA/aaRS pair (e.g., an engineered bacterial tRNA/aaRS pair) and the tRNA/aaRS pairs naturally found in the expression host cell (e.g., mammalian cell). The engineered tRNA, which is charged with the UAA via the aaRS, binds or hybridizes to the unique codon, such as a premature stop codon (U AG, UAA) present in the mRNA encoding the protein to be expressed. See, for example, FIG. 1, which shows the synthesis of a protein using an endogenous tRNA and an endogenous aaRS from the expression host cell and an engineered orthogonal tRNA and an orthogonal aaRS introduced into the host cell so as to facilitate the incorporation of a UAA into the protein as it is synthesized via the ribosome. To date, a variety of orthogonal tRNA/aaRS pairs have been produced for certain of the naturally occurring amino acids (see, e.g., U.S. Patent Publication US2017/0349891, and Zheng et al. (2018) BIOCHEM. 57:441-445). The approach facilitates the expression of proteins containing site specific modifications such as bioconjugation handles and photoactivatable crosslinkers, which can be used as therapeutics (e.g., antibody drug conjugates (ADCs), bi-specific antibodies (e.g., bispecific monoclonal antibodies), nanobodies, chemokines, vaccines, coagulation factors, hormones, and enzymes).

Although transient transfection techniques have been used to introduce engineered tRNA/aaRS pairs into expression host cells, the inability to express the proteins reproducibly, for extended periods of time, and with high titers has made this approach unsuitable for the reliable manufacture of commercial scale protein-based products. Despite the efforts made to date, there remains a need for mammalian, cell-based expression platforms that address the limitations of transient delivery of the required genetic components, to produce expression systems optimized to express proteins of interest at high titers for extended periods of time.

### SUMMARY OF THE INVENTION

The present disclosure relates, in general, to the field where orthogonal tRNA/aminoacyl-tRNA synthetase pairs are used for the incorporation of UAAs into a protein of interest as it is being synthesized. The disclosure relates to the optimization of tRNAs, aminoacyl-tRNA synthetases, and/or unnatural amino acids for use in the incorporation of unnatural amino acids into proteins, and to the construction and optimization of expression platforms (cell lines) via genome or molecular biology engineering for commercial scale production of proteins with unnatural amino acids.

In one aspect, the invention provides a prokaryotic leucyl tRNA synthetase mutein capable of charging a tRNA with an unnatural amino acid for incorporation into a protein. The tRNA synthetase mutein comprises the amino acid sequence of SEQ ID NO: 1 and (i) at least one substitution (e.g., a substitution with a hydrophobic amino acid) at a position corresponding to His537, (ii) at least one amino acid substitution selected from E20V, E20M, L41V, L41A, Y499H, Y499A, Y527I, Y527V, Y527G, and any combination thereof, (iii) at least one amino acid substitution selected from E2OK and L41S and any combination thereof and at least one amino acid substitution selected from M40I, T252A, Y499I, and Y527A, and any combination thereof, or (iv) a combination of two or more of (i), (ii) and (iii), for example, (i) and (ii), (i) and (iii), (ii) and (iii), and (i), (ii) and (iii).

In certain embodiments, the tRNA synthetase mutein may comprise E20K, M40I, L41S, T252A, Y499I, Y527A, or H537G, or any combination thereof (e.g., the tRNA synthetase mutein may comprise E20K, M40I, L41S, T252A, Y499I, Y527A, and H537G).

In certain embodiments, the leucyl-tRNA synthetase mutein comprises a substitution at position 20 with an amino acid other than a Glu or Lys, e.g., a substitution with a hydrophobic amino acid (e.g., Leu, Val, or Met). For example, the tRNA synthetase mutein may comprise: E20M, M40I, L41S, T252A, Y499I, Y527A, and H537G; or E20V, M40I, L41S, T252A, Y499I, Y527A, and H537G.

In certain embodiments, the leucyl-tRNA synthetase mutein comprises a substitution at position 41 with an amino acid other than a Leu or Ser, e.g., a substitution with a hydrophobic amino acid other than Leu (e.g., Gly, Ala, Val, or Met). For example, the tRNA synthetase mutein may comprise: E20K, M40I, L41V, T252A, Y499I, Y527A, and H537G; or E20K, M40I, L41A, T252A, Y499I, Y527A, and H537G. In certain embodiments, the tRNA synthetase mutein comprises L41V.

In certain embodiments, the leucyl-tRNA synthetase mutein comprises a substitution at position 499 with an amino acid other than a Tyr, Be or Ser, e.g., a substitution with a small hydrophobic amino acid (e.g., Gly, Ala, or Val). For example, the tRNA synthetase mutein may comprise E20K, M40I, L41S, T252A, Y499A, Y527A, and H537G. In certain embodiments, the tRNA synthetase mutein comprises a substitution at position 499 with a positively charged amino acid (e.g., Lys, Arg, or His). For example, the tRNA synthetase mutein may comprise E20K, M40I, L41S, T252A, Y499H, Y527A, and H537G.

In certain embodiments, the tRNA synthetase mutein comprises a substitution at position 527 with a hydrophobic amino acid other than Ala or Leu (e.g., Ile or Val). For example, the tRNA synthetase mutein may comprise: E20K, M40I, L41S, T252A, Y499I, Y527I, and H537G; E20K, M40I, L41S, T252A, Y499I, Y527V and H537G; or E20K, M40I,

L41S, T252A, Y499I, Y527G and H537G.

In certain embodiments, the leucyl-tRNA synthetase mutein comprises the amino acid sequence of any one of SEQ ID NOs: 2-13.

In another aspect, the invention provides a nucleic acid encoding any of the foregoing tRNA synthetase muteins.

In another aspect, the invention provides a transfer vector comprising any of the foregoing nucleic acids. In certain embodiments, the transfer vector is capable of introducing the nucleic acid into a cell. In certain embodiments, the transfer vector (or a nucleic acid from the transfer vector) can stably into the genome of the cell. In certain embodiments, the transfer vector (or a nucleic acid from the transfer vector) can be stably maintained in the cell without integration into the genome of the cell.

In another aspect, the invention provides an engineered cell comprising any of the foregoing tRNA synthetase muteins.

In another aspect, the invention provides an engineered cell comprising any of the foregoing nucleic acids, for example, where the nucleic acid is stably integrated into the genome of the cell and/or the nucleic acid is capable of being expressed in the cell to produce a corresponding tRNA synthetase mutein.

In another aspect, the invention provides an engineered cell comprising any of the foregoing transfer vectors. In certain embodiments, the transfer vector (or a nucleic acid from the transfer vector) is stably integrated into the genome of the cell. In certain embodiments, the transfer vector (or a nucleic acid from the transfer vector) is not integrated in to the genome of the cell, but is stably maintained in the cell.

In certain embodiments of any of the foregoing engineered cells, the cell further comprises a suppressor leucyl-tRNA capable of incorporating an unnatural amino acid into a protein undergoing expression in the cell. For example, the suppressor leucyl-tRNA may be selected from any one of SEQ ID NOs: 16-42. In certain embodiments, a nucleic acid encoding the suppressor leucyl-tRNA is stably integrated into the genome of the cell, and, for example, the nucleic acid is capable of being expressed in the cell to produce a corresponding suppressor tRNA.

In certain embodiments of any of the foregoing engineered cells, the unnatural amino acid is a leucine analog, for example, a leucine analog selected from a linear alkyl halide and a linear aliphatic chain comprising an alkyne, azide, cyclopropene, alkene, ketone, aldehyde, diazirine, tetrazine, or any other functional group.

In certain embodiments of any of the foregoing engineered cells, the protein is expressed from a nucleic acid sequence comprising a premature stop codon, for example, the tRNA synthetase mutein is capable of charging a suppressor leucyl tRNA with an unnatural amino acid which is incorporated into the protein at a position corresponding to the premature stop codon. In certain embodiments, the suppressor tRNA comprises an anticodon sequence that hybridizes to the premature stop codon and permits the unnatural amino to be incorporated into the protein at the position corresponding to the premature stop codon.

In certain embodiments of any of the foregoing engineered cells, the protein to be expressed in the cell is an antibody (or a fragment thereof), bispecific antibody, nanobody, affibody, viral protein, chemokine, antigen, blood coagulation factor, hormone, growth factor, enzyme, or any other polypeptide or protein.

In certain embodiments of any of the foregoing engineered cells, the cell is a prokaryotic cell (e.g., a bacterial cell) or a eukaryotic cell (e.g., a mammalian cell).

In another aspect, the invention provides a method of expressing a protein containing an unnatural amino acid. The method comprises culturing or growing any of the foregoing engineered cells under conditions that permit incorporation of the unnatural amino acid into the protein being expressed in the cell. In certain embodiments, the protein is expressed for at least 5, 10, 15, 20, 25, 30, or 35 days. In certain embodiments, the protein expressed (e.g., continuously) for at least 5, 10, 15, 20, 25, 30, or 35 days after an initial expression of the target protein.

These and other aspects and features of the invention are described in the following detailed description and claims.

### DESCRIPTION OF THE DRAWINGS

The invention can be more completely understood with reference to the following drawings.

FIG. 1 depicts a schematic overview of genetic code expansion using unnatural amino acids (UAAs).

FIGS. 2A-2C depicts a subset of UAAs that are exemplary substrates for a mutant leucyl tRNA-synthetase.

FIG. 3 depicts a subset of UAAs that are exemplary substrates for a mutant tryptophanyl tRNA-synthetase.

FIG. 4 shows the result of a leucyl tRNA synthetase mutein activity assay, using fluorescence activity as a reporter for UAA incorporation. Leucyl tRNA synthetase mutations are shown in FIG. 4A, quantified fluorescence representative of stop codon suppression and UAA incorporation is shown in FIG. 4B, and representative fluorescence images are shown in FIG. 4C.

FIG. 5 shows polyspecificity (i.e., the ability of a single synthetase to incorporate different unnatural amino acids) of the depicted leucyl tRNA synthetase muteins.

FIG. 5A depicts UAAs used in the polyspecificity assay described in Example 1, and FIG. 5B shows fluorescence images demonstrating polyspecificity.

FIG. 6 demonstrates an exemplary workflow for the generation of a stable cell line. FIG. 6A is a flowchart depicting the steps of an exemplary stable cell line generation process, and FIG. 6B is a schematic demonstrating the process from stable transfection, characterization with a dual fluorescent reporter, and integration through clonal isolation.

FIG. 7 shows fluorescence activated cell sorting (FACS) pool analysis and clonal isolation of stable Leucyl suppressor cell lines after transfection and antibiotic selection. A population shift into the 45 degree axis is indicative of changes in the conditional GFP signal and UAA incorporation. FIGS. 7A-7C depict results for controls, FIGS. 7D-7E depict results for stable cell lines obtained through lipofectamine (LF)-based transfection, and FIGS. 7F-7H depict results for stable cell lines obtained through nucleofection (NF)-based transfection. Clonal populations are identified in FIG. 71. Puromycin (Puro) concentration is indicated.

FIG. 8A and FIG. 8B show recharacterization of clonal isolates between 2-4 weeks of propagation following cell sorting via fluorescent microscopy with a conditional mCherry-GFP* reporter. GFP fluorescence, as shown in the second row of images, indicates successful incorporation of the UAA.

FIGS. 9A-9L depict FACS histograms showing the recharacterization of clonal isolates between 2-4 weeks of propagation following cell sorting with an mCherry-GFP* reporter. FIGS. 9A-9J are FACS plots of various clonal populations. FIG. 9K is a transient transfection control using the transfer vector, and FIG. 9L demonstrates a sample gate from the FACS.

FIG. 10 is a comparison of clonal suppression efficiency and protein expression in the indicated cell lines. FIG. 10A depicts average mCherry and GFP fluorescence using the conditional dual reporter across the clonal lines, FIG. 10B depicts the ratio of average mCherry and GFP fluorescence using the conditional dual reporter across the clonal lines, and FIG. 10C depicts the ratio of percentage mCherry positive cells and GFP positive cells using the conditional dual reporter across the clonal lines.

FIG. 11 is an SDS-PAGE Coomassie gel demonstrating productivity of an exemplary leucyl stable cell line, using GFP protein production as a readout of suppression activity.

FIG. 12 is a FACS comparison of stable cell line pools generated under the same selection conditions with either “wild-type” leucyl tRNA amber suppressor or h1 leucyl tRNA amber suppressor, using the conditional dual reporter as a readout. FIGS. 12A-12B are fluorescent controls, FIGS. 12C-12D show results using “wild-type” leucyl tRNA and results using h1 leucyl tRNA, respectively. FIG. 12E depicts the number of selected clones identified in each target gate P5 and P6.

FIG. 13 is a FACS pool analysis and clonal isolation of stable tryptophan suppressor cell lines after transfection and antibiotic selection. FIGS. 13A-13B represent fluorescent controls, and FIGS. 13C-13D show two different conditions for puromycin selection of tryptophan synthetase clonal isolates.

FIG. 14A depicts UAAs C5AzMe, LCA, and AzW. FIG. 14B depicts a synthetic route for C5Az. FIG. 14C depicts a synthetic route for 5-AzW. FIGS. 14D-F depict synthetic routes for LCA.

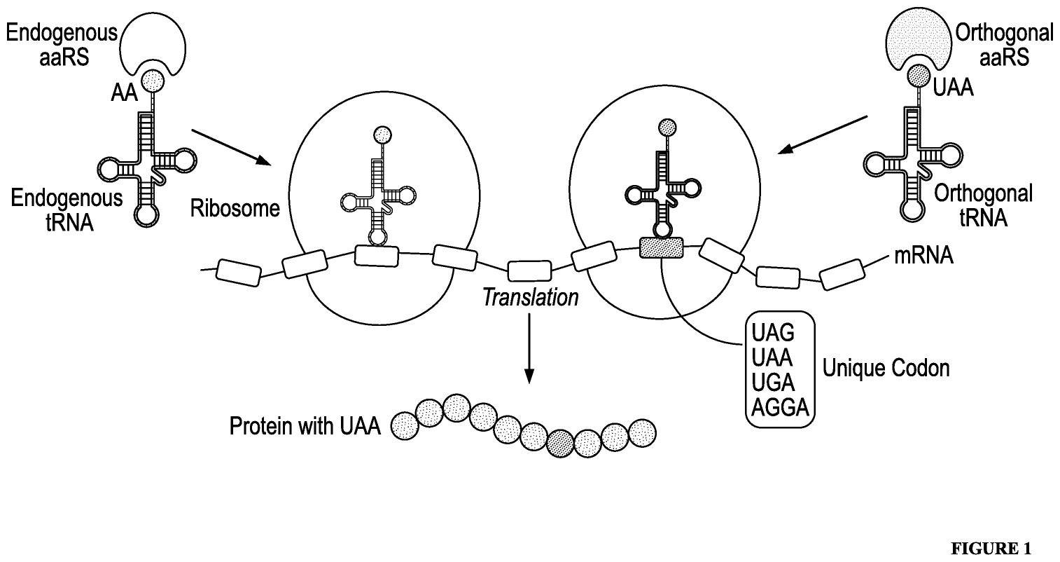

### DETAILED DESCRIPTION

The present disclosure relates, in general, to the field where orthogonal tRNA/aminoacyl-tRNA synthetase pairs are used for the incorporation of unnatural amino acids into a protein of interest. The disclosure relates to the optimization of engineered orthogonal tRNAs, engineered aminoacyl-tRNA synthetases, and/or unnatural amino acids for use in the incorporation of unnatural amino acids into proteins and to the construction and optimization of expression platforms (cell lines) via genome or molecular biology engineering for commercial scale production of proteins with unnatural amino acids.

As used herein, the term “orthogonal” refers to a molecule (e.g., an orthogonal tRNA or an orthogonal aminoacyl-tRNA synthetase) that is used with reduced efficiency by an expression system of interest (e.g., an endogenous cellular translation system). For example, an orthogonal tRNA in a translation system of interest is aminoacylated by any endogenous aminoacyl-tRNA synthetase of the translation system of interest with reduced or even zero efficiency, when compared to aminoacylation of an endogenous tRNA by an endogenous aminoacyl-tRNA synthetase. In another example, an orthogonal aminoacyl-tRNA synthetase aminoacylates any endogenous tRNA in the translation system of interest with reduced or even zero efficiency, as compared to aminoacylation of an endogenous tRNA by an endogenous aminoacyl-tRNA synthetase.

Various features and aspects of the invention are discussed in more detail below.

### I. Aminoacyl-tRNA Synthetases

The invention relates to engineered aminoacyl-tRNA synthetases (or aaRSs) capable of charging a tRNA with an unnatural amino acid for incorporation into a protein. As used herein, the term “aminoacyl-tRNA synthetase” refers to any enzyme, or a functional fragment thereof, that charges, or is capable of charging, a tRNA with an amino acid (e.g., an unnatural amino acid) for incorporation into a protein. As used herein, the term “functional fragment” of an aminoacyl-tRNA synthetase refers to fragment of a full-length aminoacyl-tRNA synthetase that retains, for example, at least 10%, at least 20%, at least 30%, at least 40%, at least 50%, at least 60%, at least 70%, at least 80%, at least 90%, at least 95%, or 100% of the enzymatic activity of the corresponding full-length tRNA synthetase (e.g., a naturally occurring tRNA synthetase). Aminoacyl-tRNA synthetase enzymatic activity may be assayed by any method known in the art. For example, in vitro aminoacylation assays are described in Hoben et al. (1985) METHODS ENZYMOL. 113:55-59 and in U.S. Patent Application Publication No. 2003/0228593 and cell-based aminoacylation assays are described in U.S. Patent Application Publication Nos. 2003/0082575 and 2005/0009049. In certain embodiments, the functional fragment comprises at least 100, 150, 200, 250, 300, 350, 400, 450, 500, 550, 600, 650, 700, 750, or 800 consecutive amino acids present in a full-length tRNA synthetase (e.g., a naturally occurring aminoacyl-tRNA synthetase).

The term aminoacyl-tRNA synthetase includes variants (i.e., muteins) having one or more mutations (e.g., amino acid substitutions, deletions, or insertions) relative to a wild-type aminoacyl-tRNA synthetase sequence. In certain embodiments, an aminoacyl-tRNA synthetase mutein may comprise, consist, or consist essentially of, a single mutation (e.g., a mutation contemplated herein), or a combination of 2, 3, 4, 5, 6, 7, 8, 9, 10, 11, 12, 13, 14, 15 or more than 15 mutations (e.g., mutations contemplated herein). It is contemplated that an aminoacyl-tRNA synthetase mutein may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 4-10, 4-7, 4-6, 4-5, 5-10, 5-7, 5-6, 6-10, 6-7, 7-10, 7-8, or 8-10 mutations (e.g., mutations contemplated herein).

An aminoacyl-tRNA synthetase mutein may comprise a conservative substitution relative to a wild-type sequence or a sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

In certain embodiments, the substrate specificity of the aminoacyl-tRNA synthetase mutein is altered relative to a corresponding (or template) wild-type aminoacyl-tRNA synthetase such that only a desired unnatural amino acid, but not any of the common 20 amino acids, is charged to the substrate tRNA.

An aminoacyl-tRNA synthetase may be derived from a bacterial source, e.g., Escherichia coli, Thermus thermophilus, or Bacillus stearothermphilus. An aminoacyl-tRNA synthetase may also be derived from an archaeal source, e.g, from the Methanosarcinacaea or Desulfitobacterium families, any of the M. barkeri (Mb), M. alvus (Ma), M. mazei (Mm) or D. hafnisense (Dh) families, Methanobacterium thermoautotrophicum, Haloferax volcanii, Halobacterium species NRC-1, or Archaeoglobus fulgidus. In other embodiments, eukaryotic sources can also be used, for example, plants, algae, protists, fungi, yeasts, or animals (e.g., mammals, insects, arthropods, etc.). As used herein, the terms “derivative” or “derived from” refer to a component that is isolated from or made using information from a specified molecule or organism. As used herein, the term “analog” refers to a component (e.g., a tRNA, tRNA synthetase, or unnatural amino acid) that is derived from or analogous with (in terms of structure and/or function) a reference component (e.g., a wild-type tRNA, a wild-type tRNA synthetase, or a natural amino acid).

In certain embodiments, derivatives or analogs have at least 40%, 50%, 60%, 70%, 80%, 90%, 100% or more of a given activity as a reference or originator component (e.g., wild type component).

It is contemplated that the aminoacyl-tRNA synthetase may aminoacylate a substrate tRNA in vitro or in vivo, and can be provided to a translation system (e.g., an in vitro translation system or a cell) as a polypeptide or protein, or as a polynucleotide that encodes the aminoacyl-tRNA synthetase.

In certain embodiments, the aminoacyl-tRNA synthetase is derived from an E. coli leucyl-tRNA synthetase and, for example, the aminoacyl-tRNA synthetase preferentially aminoacylates an E. coli leucyl tRNA (or a variant thereof) with a leucine analog over the naturally-occurring leucine amino acid.

For example, the aminoacyl-tRNA synthetase may comprise SEQ ID NO: 1, or an amino acid sequence that has at least 85%, 90%, 95%, 96%, 97%, 98%, or 99% sequence identity to SEQ ID NO: 1. In certain embodiments, the aminoacyl-tRNA synthetase comprises SEQ ID NO: 1, or a functional fragment or variant thereof, and with one, two, three, four, five or more of the following mutations: (i) a substitution of a glutamine residue at a position corresponding to position 2 of SEQ ID NO: 1, e.g., a substitution by glutamic acid (Q2E); (ii) a substitution of a glutamic acid residue at a position corresponding to position 20 of SEQ ID NO: 1, e.g., a substitution by lysine (E20K), methionine (E20M), or valine (E20V); (iii) a substitution of a methionine residue at a position corresponding to position 40 of SEQ ID NO: 1, e.g., a substitution by isoleucine (M40I) or valine (M40V); (iv) a substitution of a leucine residue at a position corresponding to position 41 of SEQ ID NO: 1, e.g., a substitution by serine (L41S), valine (L41V), or alanine (L41A); (v) a substitution of a threonine residue at a position corresponding to position 252 of SEQ ID NO: 1, e.g., a substitution by alanine (T252A) or arginine (T252R); (vi) a substitution of a tyrosine residue at a position corresponding to position 499 of SEQ ID NO: 1, e.g., a substitution by isoleucine (Y499I), serine (Y499S), alanine (Y499A), or histidine (Y499H); (vii) a substitution of a tyrosine residue at a position corresponding to position 527 of SEQ ID NO: 1, e.g., a substitution by alanine (Y527A), leucine (Y527L), isoleucine (Y527I), valine (Y527V), or glycine (Y527G); or (viii) a substitution of a histidine residue at a position corresponding to position 537 of SEQ ID NO: 1, e.g., a substitution by glycine (H537G), or any combination of the foregoing.

In certain embodiments, the aminoacyl-tRNA synthetase comprises (i) at least one substitution (e.g., a substitution with a hydrophobic amino acid) at a position corresponding to His537 of SEQ ID NO: 1, (ii) at least one amino acid substitution selected from E20V, E20M, L41V, L41A, Y499H, Y499A, Y527I, Y527V, Y527G, and any combination thereof, (iii) at least one amino acid substitution selected from E2OK and L41S and any combination thereof and at least one amino acid substitution selected from M40I, T252A, Y499I, and Y527A, and any combination thereof, or (iv) a combination of two or more of (i), (ii) and (iii), for example, (i) and (ii), (i) and (iii), (ii) and (iii) and (i), (ii) and (iii).

In certain embodiments, the aminoacyl-tRNA synthetase comprises a substitution of a glutamic acid residue at a position corresponding to position 20 of SEQ ID NO: 1, e.g., a substitution with an amino acid other than a Glu or Lys, e.g., a substitution with a hydrophobic amino acid (e.g., Leu, Val, or Met). In certain embodiments, the aminoacyl-tRNA synthetase comprises a substitution of a leucine residue at a position corresponding to position 41 of SEQ ID NO: 1, e.g., a substitution with an amino acid other than a Leu or Ser, e.g., a substitution with a hydrophobic amino acid other than Leu (e.g., Gly, Ala, Val, or Met). In certain embodiments, the aminoacyl-tRNA synthetase comprises a substitution of a tyrosine residue at a position corresponding to position 499 of SEQ ID NO: 1, e.g., a substitution with a small hydrophobic amino acid (e.g., Gly, Ala, or Val) or a substitution with a positively charged amino acid (e.g., Lys, Arg, or His). In certain embodiments, the aminoacyl-tRNA synthetase comprises a substitution of a tyrosine residue at a position corresponding to position 527 of SEQ ID NO: 1, e.g., a substitution with a hydrophobic amino acid other than Ala or Leu (e.g., Gly, Ile, Met, or Val). In certain embodiments, the tRNA synthetase mutein comprises L41V.

In certain embodiments, the aminoacyl-tRNA synthetase comprises a combination of mutations selected from: (i) Q2E, E20K, M40I, L41S, T252A, Y499I, Y527A, and H537G; (ii) Q2E, E20K, M40V, L41S, T252R, Y499S, Y527L, and H537G; (iii) Q2E, M40I, T252A, Y499I, Y527A, and H537G; (iv) Q2E, E20M, M40I, L41S, T252A, Y499I, Y527A, and H537G; (v) Q2E, E20V, M40I, L41S, T252A, Y499I, Y527A, and H537G; (vi) Q2E, E20K, M40I, L41V, T252A, Y499I, Y527A, and H537G; (vii) Q2E, E20K, M40I, L41A, T252A, Y499I, Y527A, and H537G; (viii) Q2E, E20K, M40I, L41S, T252A, Y499A, Y527A, and H537G; (ix) Q2E, E20K, M40I, L41S, T252A, Y499H, Y527A, and H537G; (x) Q2E, E20K, M40I, L41S, T252A, Y499I, Y527I, and H537G; (xi) Q2E, E20K, M40I, L41S, T252A, Y499I, Y527V, and H537G; (xii) Q2E, E20K, M40I, L41S, T252A, Y499I, Y527G, and H537G; (xiii) E20K, M40I, L41S, T252A, Y499I, Y527A, and H537G; (xiv) E20M, M40I, L41S, T252A, Y499I, Y527A, and H537G; (xv) E20V, M40I, L41S, T252A, Y499I, Y527A, and H537G; (xvi) E20K, M40I, L41V, T252A, Y499I, Y527A, and H537G; (vii) E20K, M40I, L41A, T252A, Y499I, Y527A, and H537G; (xviii) E20K, M40I, L41S, T252A, Y499A, Y527A, and H537G; (xix) E20K, M40I, L41S, T252A, Y499H, Y527A, and H537G; (xx) E20K, M40I, L41S, T252A, Y499I, Y527I, and H537G; (xxi) E20K, M40I, L41S, T252A, Y499I, Y527V, and H537G; and (xxii) E20K, M40I, L41S, T252A, Y499I, Y527G, and H537G.

In certain embodiments, the aminoacyl-tRNA synthetase comprises the amino acid sequence of any one of SEQ ID NOs: 2-13, or an amino acid sequence that has at least 85%, 90%, 95%, 96%, 97%, 98%, or 99% sequence identity to any one of SEQ ID NOs: 2-13.

In certain embodiments, the tRNA synthetase mutein comprises the amino acid sequence of SEQ ID NO: 14, wherein X₂ is Q or E, X₂₀ is E, K, V or M, X₄₀ is M, I, or V, X₄₁ is L, S, V, or A, X₂₅₂ is T, A, or R, X₄₉₉ is Y, A, I, H, or S, X₅₂₇ is Y, A, I, L, or V, and X₅₃₇ is H or G, and the tRNA synthetase mutein comprises at least one mutation (for example, 2, 3, 4, 5, 6, 7, 8, 9, or more mutations) relative to SEQ ID NO: 1. In certain embodiments, the tRNA synthetase mutein comprises the amino acid sequence of SEQ ID NO: 15, wherein X₂₀ is K, V or M, X₄₁ is S, V, or A, X₄₉₉ is A, I, or H, and X₅₂₇ is A, I, or V, and the tRNA synthetase mutein comprises at least one mutation relative to SEQ ID NO: 1.

In certain embodiments, the aminoacyl-tRNA synthetase is derived from an E. coli tryptophanyl-tRNA synthetase and, for example, the aminoacyl-tRNA synthetase preferentially aminoacylates an E. coli tryptophanyl tRNA (or a variant thereof) with a tryptophan analog over the naturally-occurring tryptophan amino acid.

For example, the aminoacyl-tRNA synthetase may comprise SEQ ID NO: 43, or an amino acid sequence that has at least 85%, 90%, 95%, 96%, 97%, 98%, or 99% sequence identity to SEQ ID NO: 43. In certain embodiments, the aminoacyl-tRNA synthetase comprises SEQ ID NO: 43, or a functional fragment or variant thereof, but with one or more of the following mutations: (i) a substitution of a serine residue at a position corresponding to position 8 of SEQ ID NO: 43, e.g., a substitution by alanine (S8A); (ii) a substitution of a valine residue at a position corresponding to position 144 of SEQ ID NO: 43, e.g., a substitution by serine (V144S), glycine (V144G) or alanine (V144A); (iii) a substitution of a valine residue at a position corresponding to position 146 of SEQ ID NO: 43, e.g., a substitution by alanine (V146A), isoleucine (V146I), or cysteine (V146C). In certain embodiments, the aminoacyl-tRNA synthetase comprises a combination of mutations selected from: (i) S8A, V144S, and V146A, (ii) S8A, V144G, and V1461, (iii) S8A, V144A, and V146A, and (iv) S8A, V144G, and V146C.

In certain embodiments, the aminoacyl-tRNA synthetase comprises the amino acid sequence of any one of SEQ ID NOs: 44-47, or an amino acid sequence that has at least 85%, 90%, 95%, 96%, 97%, 98%, or 99% sequence identity to any one of SEQ ID NOs: 44-47.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default =2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; −X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

Methods for producing proteins, e.g., aminoacyl-tRNA synthetases, are known in the art. For example, DNA molecules encoding a protein of interest can be synthesized chemically or by recombinant DNA methodologies. The resulting DNA molecules encoding the protein interest can be ligated to other appropriate nucleotide sequences, including, for example, expression control sequences, to produce conventional gene expression constructs (i.e., expression vectors) encoding the desired protein. Production of defined gene constructs is within routine skill in the art.

Nucleic acids encoding desired proteins (e.g, aminoacyl-tRNA synthetases) can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the desired protein.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. The gene construct can be introduced into eukaryotic host cells using conventional techniques.

A protein of interest (e.g, an aminoacyl-tRNA synthetase) can be produced by growing (culturing) a host cell transfected with an expression vector encoding such a protein under conditions that permit expression of the protein. Following expression, the protein can be harvested and purified or isolated using techniques known in the art, e.g., affinity tags such as glutathione-S-transferase (GST) or histidine tags.

Additional methods for producing aminoacyl-tRNA synthetases, and for altering the substrate specificity of the synthetase can be found in U.S. Patent Application Publication Nos. 2003/0108885 and 2005/0009049, Hamano-Takaku et al. (2000) JOURNAL OF BIOL. CHEM. 275(51):40324-40328, Kiga et al. (2002) PROC. NATL. ACAD. SCI. USA 99(15): 9715-9723, and Francklyn et al. (2002) RNA, 8:1363-1372.

The invention also encompasses nucleic acids encoding aminoacyl-tRNA synthetases disclosed herein. For example, nucleotide sequences encoding leucyl-tRNA synthetase muteins disclosed herein are depicted in SEQ ID NOs: 55-66. Accordingly, the invention provides a nucleic acid comprising the nucleotide sequence of any one of SEQ ID NOs: 55-66, or a nucleotide sequence that has at least 85%, 90%, 95%, 96%, 97%, 98%, or 99% sequence identity to any one of SEQ ID NOs: 55-66. The invention also provides a nucleic acid comprising a nucleotide sequence encoding the amino acid sequence encoded by any one of SEQ ID NOs: 55-66, or a nucleotide sequence that has at least 85%, 90%, 95%, 96%, 97%, 98%, or 99% sequence identity to a nucleotide sequence encoding the amino acid sequence encoded by any one of SEQ ID NOs: 55-66.

### II. tRNAs

The invention relates to transfer RNAs (tRNAs) that mediate the incorporation of unnatural amino acids into proteins.

During protein synthesis, a tRNA molecule delivers an amino acid to a ribosome for incorporation into a growing protein (polypeptide) chain. tRNAs typically are about 70 to 100 nucleotides in length. Active tRNAs contain a 3′ CCA sequence that may be transcribed into the tRNA during its synthesis or may be added later during post-transcriptional processing. During aminoacylation, the amino acid that is attached to a given tRNA molecule is covalently attached to the 2′ or 3′ hydroxyl group of the 3′-terminal ribose to form an aminoacyl-tRNA (aa-tRNA). It is understood that an amino acid can spontaneously migrate from the 2′-hydroxyl group to the 3′-hydroxyl group and vice versa, but it is incorporated into a growing protein chain at the ribosome from the 3′-OH position. A loop at the other end of the folded aa-tRNA molecule contains a sequence of three bases known as the anticodon. When this anticodon sequence hybridizes or base-pairs with a complementary three-base codon sequence in a ribosome-bound mRNA, the aa-tRNA binds to the ribosome and its amino acid is incorporated into the polypeptide chain being synthesized by the ribosome. Because all tRNAs that base-pair with a specific codon are aminoacylated with a single specific amino acid, the translation of the genetic code is effected by tRNAs. Each of the 61 non-termination codons in an mRNA directs the binding of its cognate aa-tRNA and the addition of a single specific amino acid to the growing polypeptide chain being synthesized by the ribosome. The term “cognate” refers to components that function together, e.g., a tRNA and an aminoacyl-tRNA synthetase.

Suppressor tRNAs are modified tRNAs that alter the reading of a mRNA in a given translation system. For example, a suppressor tRNA may read through a codon such as a stop codon, a four base codon, or a rare codon. The use of the word in suppressor is based on the fact, that under certain circumstance, the modified tRNA “suppresses” the typical phenotypic effect of the codon in the mRNA. Suppressor tRNAs typically contain a mutation (modification) in either the anticodon, changing codon specificity, or at some position that alters the aminoacylation identity of the tRNA. The term “suppression activity” refers to the ability of a tRNA, e.g., a suppressor tRNA, to read through a codon (e.g., a premature stop codon) that would not be read through by the endogenous translation machinery in a system of interest.

In certain embodiments, a tRNA (e.g., a suppressor tRNA) contains a modified anticodon region, such that the modified anticodon hybridizes with a different codon than the corresponding naturally occurring anticodon.

In certain embodiments, a tRNA comprises an anticodon that hybridizes to a codon selected from UAG (i.e., an “amber” termination codon), UGA (i.e., an “opal” termination codon), and UAA (i.e., an “ochre” termination codon).

In certain embodiments, a tRNA comprises an anticodon that hybridizes to a non-standard codon, e.g., a 4- or 5-nucleotide codon. Examples of four base codons include AGGA, CUAG, UAGA, and CCCU. Examples of five base codons include AGGAC, CCCCU, CCCUC, CUAGA, CUACU, and UAGGC. tRNAs comprising an anticodon that hybridizes to a non-standard codon, e.g., a 4- or 5-nucleotide codon, and methods of using such tRNAs to incorporate unnatural amino acids into proteins are described, for example, in Moore et al. (2000) J. MOL. BIOL. 298:195; Hohsaka et al. (1999) J. AM. CHEM. SOC. 121:12194; Anderson et al. (2002) CHEMISTRY AND BIOLOGY 9:237-244; Magliery (2001) J. MOL. BIOL. 307: 755-769; and PCT Publication No. WO2005/007870.

As used herein, the term “tRNA” includes variants having one or more mutations (e.g., nucleotide substitutions, deletions, or insertions) relative to a reference (e.g., a wild-type) tRNA sequence. In certain embodiments, a tRNA may comprise, consist, or consist essentially of, a single mutation (e.g., a mutation contemplated herein), or a combination of 2, 3, 4, 5, 6, 7, 8, 9, 10, 11, 12, 13, 14, 15 or more than 15 mutations (e.g., mutations contemplated herein). It is contemplated that a tRNA may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 mutations (e.g., mutations contemplated herein).

In certain embodiments, a variant suppressor tRNA has increased activity to incorporate an unnatural amino acid (e.g., an unnatural amino acid contemplated herein) into a mammalian protein relative to a counterpart wild-type suppressor tRNA (in this context, a wild-type suppressor tRNA refers to a suppressor tRNA that corresponds to a wild-type tRNA molecule but for any modifications to the anti-codon region to impart suppression activity). The activity of the variant suppressor tRNA may be increased relative to the wild type suppressor tRNA, for example, by about 2.5 to about 200 fold, about 2.5 to about 150 fold, about 2.5 to about 100 fold about 2.5 to about 80 fold, about 2.5 to about 60 fold, about 2.5 to about 40 fold, about 2.5 to about 20 fold, about 2.5 to about 10 fold, about 2.5 to about 5 fold, about 5 to about 200 fold, about 5 to about 150 fold, about 5 to about 100 fold, about 5 to about 80 fold, about 5 to about 60 fold, about 5 to about 40 fold, about 5 to about 20 fold, about 5 to about 10 fold, about 10 to about 200 fold, about 10 to about 150 fold, about 10 to about 100 fold, about 10 to about 80 fold, about 10 to about 60 fold, about 10 to about 40 fold, about 10 to about 20 fold, about 20 to about 200 fold, about 20 to about 150 fold, about 20 to about 100 fold, about 20 to about 80 fold, about 20 to about 60 fold, about 20 to about 40 fold, about 40 to about 200 fold, about 40 to about 150 fold, about 40 to about 100 fold, about 40 to about 80 fold, about 40 to about 60 fold, about 60 to about 200 fold, about 60 to about 150 fold, about 60 to about 100 fold, about 60 to about 80 fold, about 80 to about 200 fold, about 80 to about 150 fold, about 80 to about 100 fold, about 100 to about 200 fold, about 100 to about 150 fold, or about 150 to about 200 fold.

It is contemplated that the tRNA may function in vitro or in vivo and can be provided to a translation system (e.g., an in vitro translation system or a cell) as a mature tRNA (e.g., an aminoacylated tRNA), or as a polynucleotide that encodes the tRNA.

A tRNA may be derived from a bacterial source, e.g., Escherichia coli, Thermus thermophilus, or Bacillus stearothermphilus. A tRNA may also be derived from an archaeal source, e.g, from the Methanosarcinacaea or Desulfitobacterium families, any of the M. barkeri (Mb), M. alvus (Ma), M. mazei (Mm) or D. hafnisense (Dh) families, Methanobacterium thermoautotrophicum, Haloferax volcanii, Halobacterium species NRC-1, or Archaeoglobus fulgidus. In other embodiments, eukaryotic sources can also be used, for example, plants, algae, protists, fungi, yeasts, or animals (e.g., mammals, insects, arthropods, etc.).

In certain embodiments, the tRNA is derived from an E. coli leucyl tRNA and, for example, is preferentially charged with a leucine analog over the naturally-occurring leucine amino acid by an aminoacyl-tRNA synthetase derived from an E. coli leucyl-tRNA synthetase, e.g., an aminoacyl-tRNA synthetase contemplated herein.

For example, the tRNA may comprise, consist essentially of, or consist of the nucleotide sequence of any one of SEQ ID NOs: 16-42, or a nucleotide sequence that has at least 85%, 90%, 95%, 96%, 97%, 98%, or 99% sequence identity to any one of SEQ ID NOs: 16-42.

In certain embodiments, the tRNA is derived from an E. coli tryptophanyl tRNA and, for example, is preferentially charged with a tryptophan analog over the naturally-occurring tryptophan amino acid by an aminoacyl-tRNA synthetase derived from an E. coli tryptophanyl-tRNA synthetase, e.g., an aminoacyl-tRNA synthetase contemplated herein.

For example, the tRNA may comprise, consist essentially of, or consist of the nucleotide sequence of any one of SEQ ID NOs: 49-53, or a nucleotide sequence that has at least 85%, 90%, 95%, 96%, 97%, 98%, or 99% sequence identity to any one of SEQ ID NOs: 49-53.

It is understood that, throughout the description, in each instance where a tRNA comprises, consists essentially of, or consists of a nucleotide sequence including one or more thymines (T), a tRNA is also contemplated that comprises, consists essentially of, or consists of the same nucleotide sequence including a uracil (U) in place of one or more of the thymines (T), or a uracil (U) in place of all the thymines (T). Similarly, in each instance where a tRNA comprises, consists essentially of, or consists of a nucleotide sequence including one or more uracils (U), a tRNA is also contemplated that comprises, consists essentially of, or consists of a nucleotide sequence including a thymine (T) in place of the one or more of the uracils (U), or a thymine (T) in place of all the uracils (U). In addition, additional modifications to the bases can be present.

Methods for producing recombinant tRNA are described in U.S. Patent Application Publication Nos. 2003/0108885 and 2005/0009049, Forster et al. (2003) PROC. NATL. ACAD. SCI. USA 100(11):6353-6357, and Feng et al. (2003), PROC. NATL. ACAD. SCI. USA 100(10): 5676-5681.

A tRNA may be aminoacylated (i.e., charged) with a desired unnatural amino acid (UAA) by any method, including enzymatic or chemical methods.

Enzymatic molecules capable of charging a tRNA include aminoacyl-tRNA synthetases, e.g., aminoacyl-tRNA synthetases disclosed herein. Additional enzymatic molecules capable of charging tRNA include ribozymes, for example, as described in Illangakekare et al. (1995) SCIENCE 267:643-647, Lohse et al. (1996) NATURE 381:442-444, Murakami et al. (2003) CHEMISTRY AND BIOLOGY 10:1077-1084, U.S. Patent Application Publication No. 2003/0228593,

Chemical aminoacylation methods include those described in Hecht (1992) ACC. CHEM. RES. 25:545, Heckler et al. (1988) BIOCHEM. 1988, 27:7254, Hecht et al. (1978) J. BIOL. CHEM. 253:4517, Cornish et al. (1995) ANGEW. CHEM. INT. ED. ENGL. 34:621, Robertson et al. (1991) J. AM. CHEM. SOC. 113:2722, Noren et al. (1989) SCIENCE 244:182, Bain et al. (1989) J. AM. CHEM. SOC. 111:8013, Bain et al. (1992) NATURE 356:537, Gallivan et al. (1997) CHEM. BIOL. 4:740, Turcatti et al. (1996) J. BIOL. CHEM. 271:19991, Nowak et al. (1995) SCIENCE 268:439, Saks et al. (1996) J. BIOL. CHEM. 271:23169, and Hohsaka et al. (1999) J. AM. CHEM. SOC. 121:34.

### III. Unnatural Amino Acids (UAAs)

The invention relates to unnatural amino acids (UAAs) and their incorporation into proteins.

As used herein, an unnatural amino acid refers to any amino acid, modified amino acid, or amino acid analogue other than the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. See, e.g., Biochemistry by L. Stryer, 3rd ed. 1988, Freeman and Company, New York, for structures of the twenty natural amino acids. The term unnatural amino acid also includes amino acids that occur by modification (e.g. post-translational modifications) of a natural amino acid but are not themselves naturally incorporated into a growing polypeptide chain by the translation complex.

Because unnatural amino acids typically differ from natural amino acids only in the structure of the side chain, unnatural amino acids may, for example, form amide bonds with other amino acids in the same manner in which they are formed in naturally occurring proteins. However, the unnatural amino acids have side chain groups that distinguish them from the natural amino acids. For example, the side chain may comprise an alkyl-, aryl-, acyl-, keto-, azido-, hydroxyl-, hydrazine, cyano-, halo-, hydrazide, alkenyl, alkyl, ether, thiol, seleno-, sulfonyl-, borate, boronate, phospho, phosphono, phosphine, heterocyclic, enone, imine, aldehyde, ester, thioacid, hydroxylamine, amine, and the like, or any combination thereof. Other non-naturally occurring amino acids include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto-containing amino acids, amino acids comprising polyethylene glycol or polyether, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

In addition to unnatural amino acids that contain novel side chains, unnatural amino acids also optionally comprise modified backbone structures.

Many unnatural amino acids are based on natural amino acids, such as tyrosine, glutamine, phenylalanine, and the like. Tyrosine analogs include para-substituted tyrosines, ortho-substituted tyrosines, and meta substituted tyrosines, wherein the substituted tyrosine comprises a keto group (including but not limited to, an acetyl group), a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, or the like. In addition, multiply substituted aryl rings are also contemplated. Glutamine analogs include, but are not limited to, α-hydroxy derivatives, γ-substituted derivatives, cyclic derivatives, and amide substituted glutamine derivatives. Exemplary phenylalanine analogs include, but are not limited to, para-substituted phenylalanines, ortho-substituted phenylalanines, and meta-substituted phenylalanines, wherein the substituent comprises a hydroxy group, a methoxy group, a methyl group, an allyl group, an aldehyde, an azido, an iodo, a bromo, a keto group (including but not limited to, an acetyl group), or the like. Specific examples of unnatural amino acids include, but are not limited to, a p-acetyl-L-phenylalanine, a p-propargyl-phenylalanine, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine, and the like.

Examples of structures of a variety of unnatural amino acids are provided in U.S. Patent Application Publication Nos. 2003/0082575 and 2003/0108885, PCT Publication No. WO 2002/085923, and Kiick et al. (2002) PROC. NATL. ACAD. SCI. USA 99:19-24.

An unnatural amino acid in a polypeptide may be used to attach another molecule to the polypeptide, including but not limited to, a label, a dye, a polymer, a water-soluble polymer, a derivative of polyethylene glycol, a photoactivatable crosslinker, a radionuclide, a cytotoxic compound, a drug, an affinity label, a photoaffinity label, a reactive compound, a resin, a second protein or polypeptide or polypeptide analog, an antibody or antibody fragment, a metal chelator, a cofactor, a fatty acid, a carbohydrate, a polynucleotide, a DNA, a RNA, an antisense polynucleotide, a saccharide, a water-soluble dendrimer, a cyclodextrin, an inhibitory ribonucleic acid, a biomaterial, a nanoparticle, a spin label, a fluorophore, a metal-containing moiety, a radioactive moiety, a novel functional group, a group that covalently or noncovalently interacts with other molecules, a photocaged moiety, an actinic radiation excitable moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, a quantum dot, a nanotransmitter, an immunomodulatory molecule, or any combination of the above.

Any suitable unnatural amino acid can be used with the methods described herein for incorporation into a protein of interest.

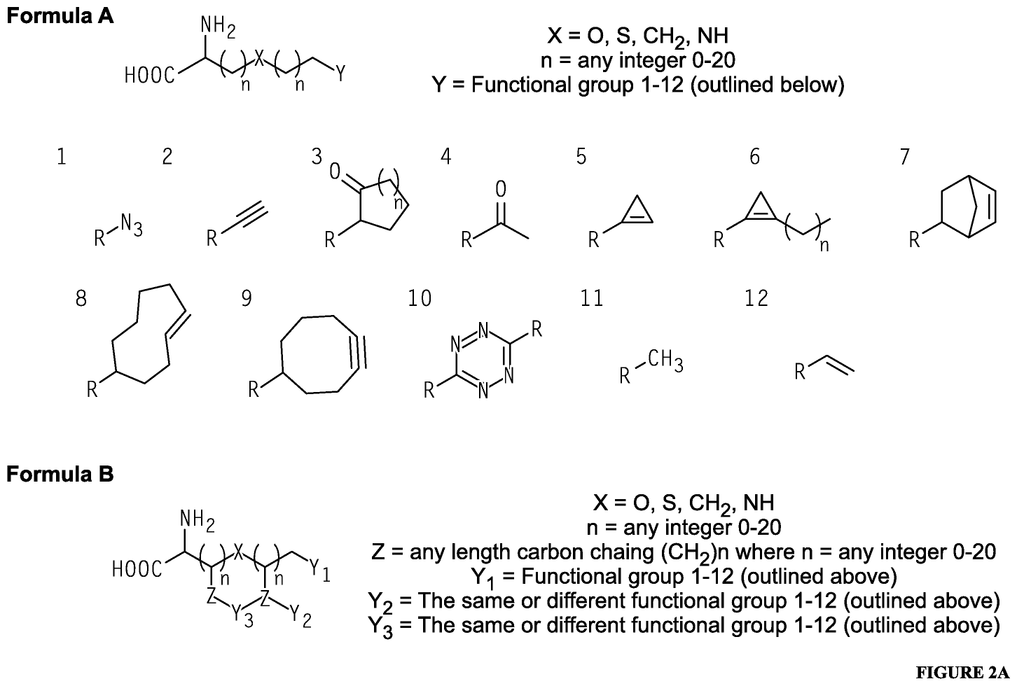

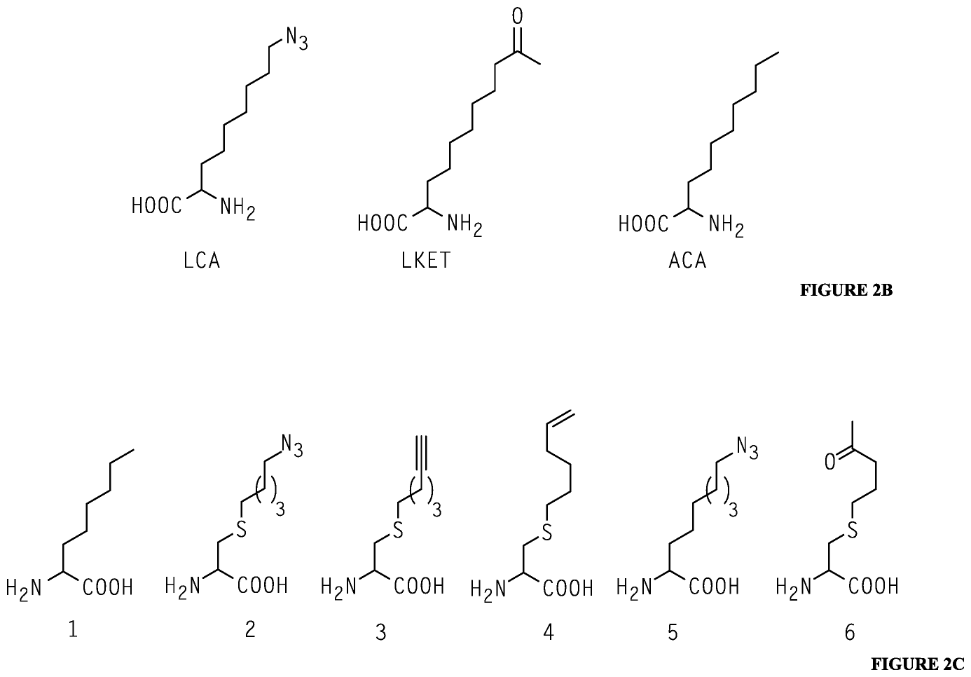

The unnatural amino acid may be a leucine analog. The invention provides a leucine analog depicted in FIG. 2A, or a composition comprising the leucine analog. For example, Formula A in FIG. 2A depicts an amino acid analog containing a side chain including a carbon containing chain n units (0-20 units) long. An O, S, CH₂, or NH is present in at position X, and another carbon containing chain of n units (0-20 units) long can follow. A functional group Y is attached to the terminal carbon of second carbon containing chain (for example, functional groups 1-12 as depicted in FIG. 2A, where R represents a linkage to the terminal carbon atom the second carbon containing side chain). In one example, these functional groups can be used for bioconjugation of any amenable ligand to any protein of interest that is amenable to site-specific UAA incorporation. Formula B in FIG. 2A depicts a similar amino acid analog containing an side chains denoted as either Z-Y₂ or Z-Y₃ attached to the second carbon containing chain or the first carbon containing chain, respectively. Z represents a carbon chain comprising (CH₂)n units, where n is any integer from 0-20. Y₂ or Y₃, independently, can be the same or different groups as those of Yi. The invention also provides a leucine analog depicted in FIG. 2B (LCA, LKET, or ACA), or a composition comprising the leucine analog depicted in FIG. 2B. Additional exemplary leucine analogs include those selected from linear alkyl halides and linear aliphatic chains comprising a functional group, for example, an alkyne, azide, cyclopropene, alkene, ketone, aldehyde, diazirine, or tetrazine functional group, as well as structures 1-6 shown in FIG. 2C. However, it is contemplated that the amino and carboxylate groups both attached to the first carbon of any amino acid shown in FIGS. 2A-2C would constitute portions of peptide bonds when the leucine analog is incorporated into a protein or polypeptide chain.

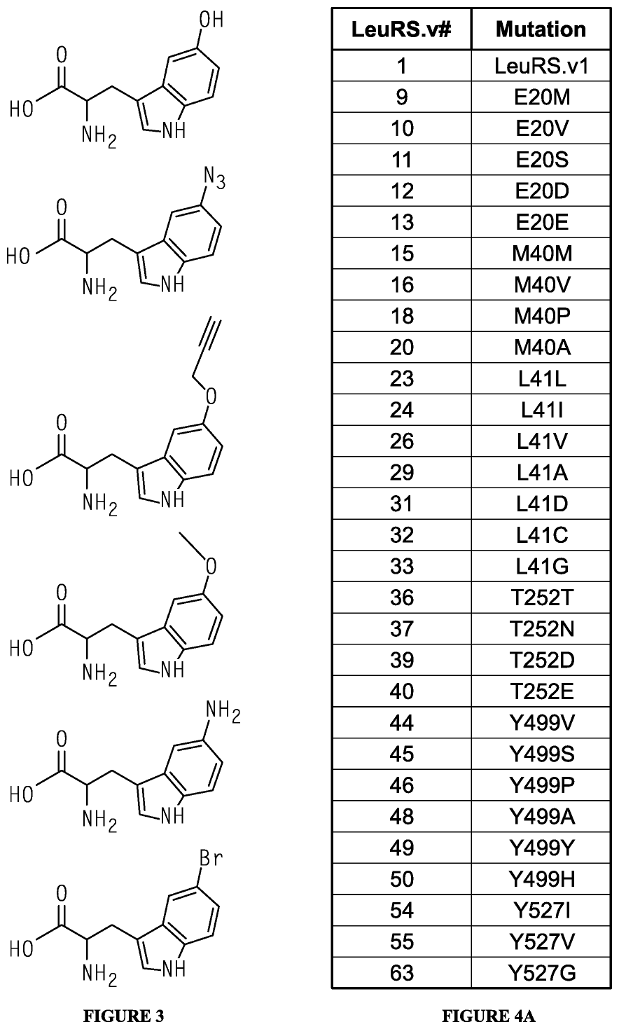

In certain embodiments, the unnatural amino acid is a tryptophan analog (also referred to herein as a derivative). Exemplary tryptophan analogs include 5-azidotryptophan, 5-propargyloxytryptophan, 5-aminotryptophan, 5-methoxytryptophan, 5-O-allyltryptophan or 5-bromotryptophan. Additional exemplary tryptophan analogs are depicted in FIG. 3. However, it is contemplated that the amino and carboxylate groups both attached to the first carbon of the tryptophan analogs in FIG. 3 would constitute portions of peptide bonds when the tryptophan analog is incorporated into a protein or polypeptide chain.

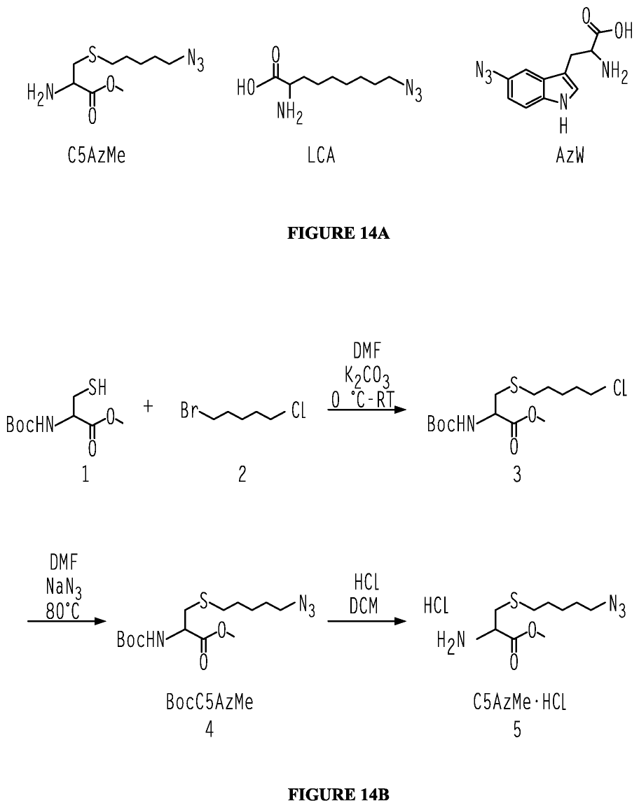

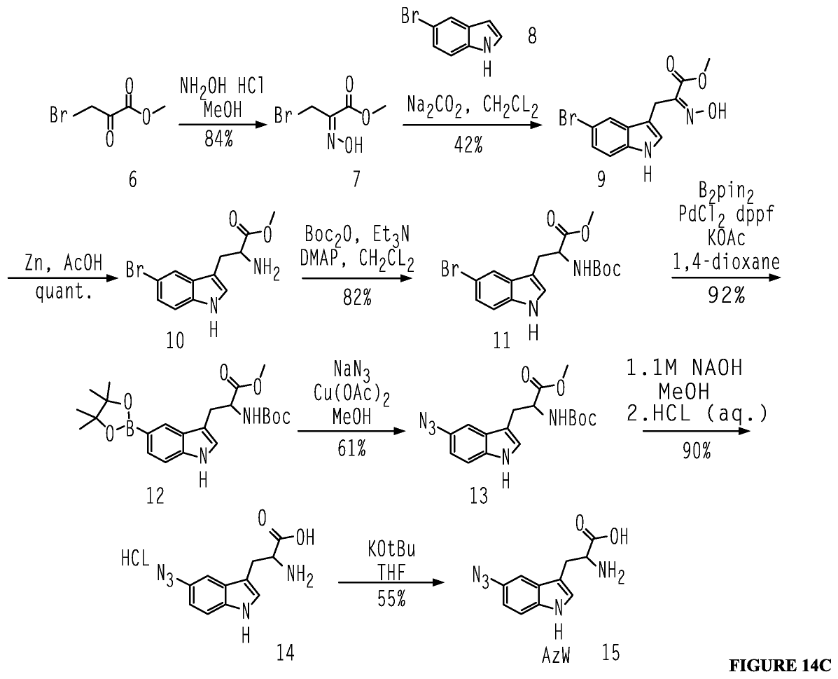

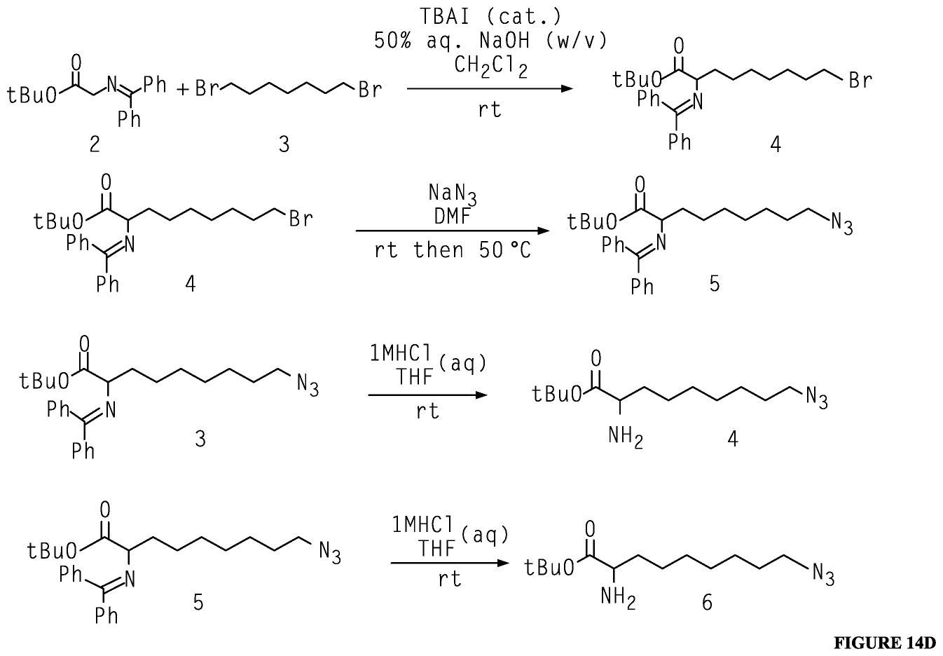

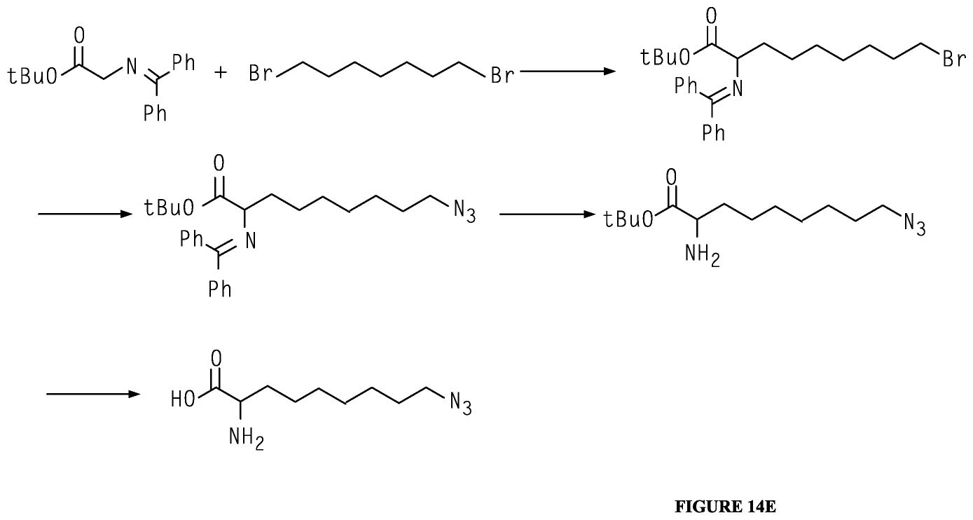

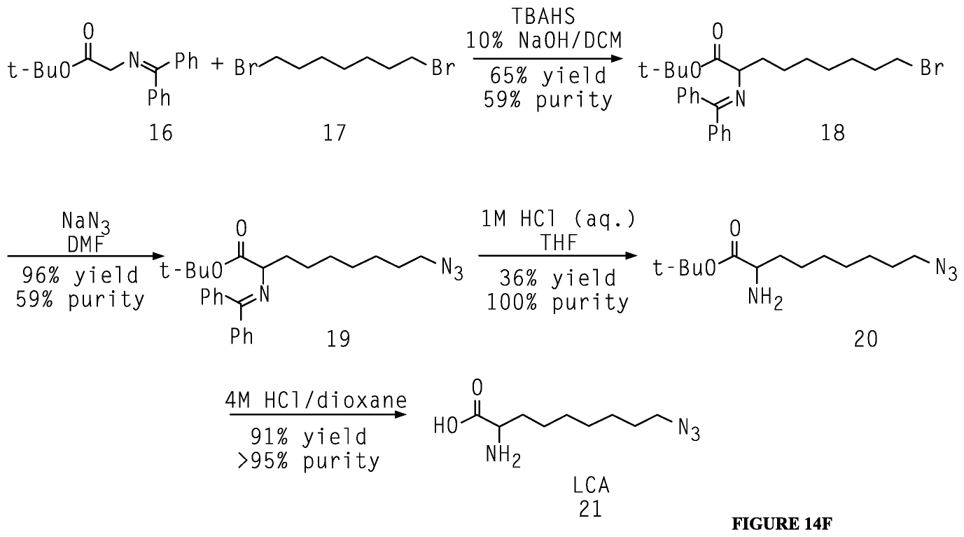

In addition, the UAAs set forth in FIG. 14A, referred to as C5AzMe (a leucyl analog), LCA (a leucyl analog) and AzW (a tryptophan analog) can be used in the practice of the invention.

C5AzMe (Compound 5 as shown in FIG. 14B) can be prepared in a manner similar to the synthesis outlined in FIG. 14B. Compound 5 can be furnished by, for example, the deprotection of Compound 4. Deprotection of Compound 4 comprises the removal of a protecting group (e.g. Boc). Conditions for deprotection may include, but are not limited to, HCl in DCM. Compound 4 can be generated, for example, via nucleophilic substitution of Compound 3 when exposed to a suitable nucleophile (e.g. N₃⁻). Exemplary conditions for nucleophilic substitution include, but are not limited to, NaN₃ in DMF at 80° C. Compound 3 can be prepared, for example, via nucleophilic addition of Compound 1 to Compound 2. Exemplary conditions for nucleophilic addition include, but are not limited to, K₂CO₃ at 0° C. to RT. Furthermore, if desired, the ester of Compound 5 can be removed by exposure to mild aqueous basic conditions to produce the carboxylic acid form of the UAA.

AzW (Compound 15 as shown in FIG. 14C) can be prepared in a manner similar to the synthesis outlined in FIG. 14C. Compound 15 can be prepared, for example, under basic conditions from its hydrochloride salt 14. Exemplary basic conditions include, but are not limited to, KOtBu in THF. Hydrochloride salt 14 can be prepared, for example, via saponification followed by deprotection of Compound 13. Conditions for saponification and deprotection of a protecting group (e.g., Boc) are known to a person of ordinary skill in the art. For example, saponification can be accomplished using 1M NaOH in MeOH. In certain embodiments, conditions for deprotection include, but are not limited to, HCl (aq.). Compound 13 can be synthesized, for example, through a metal-mediated coupling of Compound 12 with a suitable azide source. Compound 13 can be made, for example, from Compound 12 using NaN₃, Cu(OAc)₂ in MeOH. Compound 12 can, for example, be prepared from Compound 11 through metal-catalyzed boronation of Compound 11. Exemplary conditions for metal-catalyzed boronation include, but are not limited to B₂pin₂, PdCl₂·dppf, and KOAc in 1,4-dioxane. Compound 11 can be prepared, for example, via protection of Compound 10 using a suitable protecting group (e.g., Boc). Protection of Compound 10 can be accomplished using Boc₂O, Et₃N, and DMAP in CH₂Cl₂. Compound 10 can be synthesized, for example, from Compound 9 under conditions suitable for reducing an oxime, for example, using Zn in AcOH. Compound 9 can synthesized, for example, via nucleophilic addition of indole 8 to Compound 7. Narcoleptic addition of Compound 8 to Compound 7 can occur in the presence of Na₂CO₃ in CH₂Cl₂. Compound 7 can be prepared, for example, by exposing Compound 6 to hydroxylamine hydrochloride in methanol.

LCA (Compound 21 as shown in FIG. 14F) can be prepared in a manner similar to the synthesis outlined in FIG. 14F. Compound 21 can be prepared, for example, from Compound 20 through exposure of Compound 20 to a suitable acid, for example, but not limited to, 4M HCl in dioxane. Compound 20 can be generated through hydrolyzation of imine 19. Hydrolyzation of imine 19 can be accomplished, for example, using 1M HCl (aq.) in THF. Compound 19 can be generated, for example, via nucleophilic substitution of Compound 18 when exposed to a suitable nucleophile (e.g. N₃⁻). Exemplary conditions for nucleophilic substitution include, but are not limited to, NaN₃ in DMF. Compound 18 can be prepared via nucleophilic addition of the enolate of Compound 16 to Compound 17. Suitable conditions for accomplishing synthesis of compound from Compounds 16 and 17 include, but are not limited to, tetrabutylammonium hydrogensulfate (TBAHS) and 10% NaOH in DCM. Additional methods for synthesis of LCA are shown in FIGS. 14D and 14E.

Many unnatural amino acids are commercially available, e.g., from Sigma-Aldrich (St. Louis, Mo., USA), Novabiochem (Darmstadt, Germany), or Peptech (Burlington, Mass., USA). Those that are not commercially available can be synthesized using standard methods known to those of ordinary skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional exemplary publications describing the synthesis of unnatural amino acids appear in PCT Publication No. WO2002/085923, U.S. Patent Application Publication No. 2004/0198637, Matsoukas et al. (1995) J. MED. CHEM. 38:4660-4669, King et al. (1949) J. CHEM. SOC. 3315-3319, Friedman et al. (1959) J. AM. CHEM. SOC. 81:3750-3752, Craig et al. (1988) J. ORG. CHEM. 53:1167-1170, Azoulay et al. (1991) EUR. J. MED. CHEM. 26:201-5, Koskinen et al. (1989) J. ORG. CHEM. 54:1859-1866, Christie et al. (1985) J. ORG. CHEM. 50:1239-1246, Barton et al. (1987) TETRAHEDRON 43:4297-4308, and Subasinghe et al. (1992) J. MED. CHEM. 35:4602-7.

### IV. Vectors

tRNAs, aminoacyl-tRNA synthetases, or any other molecules of interest may be expressed in a cell of interest by incorporating a gene encoding the molecule into an appropriate expression vector. As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system.

tRNAs, aminoacyl-tRNA synthetases, or any other molecules of interest may be introduced to a cell of interest by incorporating a gene encoding the molecule into an appropriate transfer vector. The term “transfer vector” refers to a vector comprising a recombinant polynucleotide which can be used to deliver the polynucleotide to the interior of a cell. It is understood that a vector may be both an expression vector and a transfer vector.

Vectors (e.g., expression vectors or transfer vectors) include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both (including but not limited to, shuttle vectors) and selection markers for both prokaryotic and eukaryotic systems.

In certain embodiments, the vector comprises a regulatory sequence or promoter operably linked to the nucleotide sequence encoding the suppressor tRNA and/or the tRNA synthetase. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Exemplary promoters which may be employed include, but are not limited to, the retroviral LTR, the SV40 promoter, the human cytomegalovirus (CMV) promoter, the U6 promoter, the EF1α promoter, the CAG promoter, the H1 promoter, the UbiC promoter, the PGK promoter, the 7SK promoter, a pol II promoter, a pol III promoter, or any other promoter (e.g., cellular promoters such as eukaryotic cellular promoters including, but not limited to, the histone, pol III, and (3-actin promoters). Other viral promoters which may be employed include, but are not limited to, adenovirus promoters, TK promoters, and B19 parvovirus promoters. The selection of a suitable promoter will be apparent to those skilled in the art from the teachings contained herein. In certain embodiments, a vector comprises a nucleotide sequence encoding an aminacyl-tRNA synthetase operably linked to a CMV or an EF1α promoter and/or a nucleotide sequence encoding a suppressor tRNA operably linked to a U6 or an H1 promoter.

In certain embodiments, the vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyorriavirus vectors (e.g., simian vacuolating virus 40 (SV40) vectors), poxvirus vectors, and pseudotype virus vectors.

The virus may be a RNA virus (having a genome that is composed of RNA) or a DNA virus (having a genome composed of DNA). In certain embodiments, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HS V-2), epstein-barr virus (EMI), cytomegalovirus (CMV)), papillomoviruses (e.g., HPV), polyomaviruses (e.g., simian vacuolating virus 40 (SV40)), and poxviruses vaccinia virus, cowpox virus, smallpox virus, fowlpox virus, sheeppox virus, myxoma virus). In certain embodiments, the viral vector is a RNA virus vector. Exemplary RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

Adeno-Associated Virus (AAV) Vectors

In certain embodiments, a vector is an adeno-associated virus (AAV) vector. AAV is a small, nonenveloped icosahedral virus of the genus Dependoparvovirus and family Parvovirus. AAV has a single-stranded linear DNA genome of approximately 4.7 kb. AAV is capable of infecting both dividing and quiescent cells of several tissue types, with different AAV serotypes exhibiting different tissue tropism.

AAV includes numerous serologically distinguishable types including serotypes AAV-1 to AAV-12, as well as more than 100 serotypes from nonhuman primates (See, e.g., Srivastava (2008) J. CELL BIOCHEM., 105(1): 17-24, and Gao et al. (2004) J. VIROL., 78(12), 6381-6388). The serotype of the AAV vector used in the present invention can be selected by a skilled person in the art based on the efficiency of delivery, tissue tropism, and immunogenicity. For example, AAV-1, AAV-2, AAV-4, AAV-5, AAV-8, and AAV-9 can be used for delivery to the central nervous system; AAV-1, AAV-8, and AAV-9 can be used for delivery to the heart; AAV-2 can be used for delivery to the kidney; AAV-7, AAV-8, and AAV-9 can be used for delivery to the liver; AAV-4, AAV-5, AAV-6, AAV-9 can be used for delivery to the lung, AAV-8 can be used for delivery to the pancreas, AAV-2, AAV-5, and AAV-8 can be used for delivery to the photoreceptor cells; AAV-1, AAV-2, AAV-4, AAV-5, and AAV-8 can be used for delivery to the retinal pigment epithelium; AAV-1, AAV-6, AAV-7, AAV-8, and AAV-9 can be used for delivery to the skeletal muscle. In certain embodiments, the AAV capsid protein comprises a sequence as disclosed in U.S. Pat. No. 7,198,951, such as, but not limited to, AAV-9 (SEQ ID NOs: 1-3 of U.S. Pat. No. 7,198,951), AAV-2 (SEQ ID NO: 4 of U.S. Pat. No. 7,198,951), AAV-1 (SEQ ID NO: 5 of U.S. Pat. No. 7,198,951), AAV-3 (SEQ ID NO: 6 of U.S. Pat. No. 7,198,951), and AAV-8 (SEQ ID NO: 7 of U.S. Pat. No. 7,198,951). AAV serotypes identified from rhesus monkeys, e.g., rh.8, rh.10, rh.39, rh.43, and rh.74, are also contemplated in the instant invention. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous gene of interest. Accordingly, in certain embodiments, the AAV vector comprises a genome comprising an expression cassette for an exogenous gene flanked by a 5′ ITR and a 3′ ITR. The ITRs may be derived from the same serotype as the capsid or a derivative thereof. Alternatively, the ITRs may be of a different serotype from the capsid, thereby generating a pseudotyped AAV. In certain embodiments, the ITRs are derived from AAV-2. In certain embodiments, the ITRs are derived from AAV-5. At least one of the ITRs may be modified to mutate or delete the terminal resolution site, thereby allowing production of a self-complementary AAV vector.

The rep and cap proteins can be provided in trans, for example, on a plasmid, to produce an AAV vector. A host cell line permissive of AAV replication must express the rep and cap genes, the ITR-flanked expression cassette, and helper functions provided by a helper virus, for example adenoviral genes E1a, E1b55K, E2a, E4orf6, and VA (Weitzman et al., Adeno-associated virus biology. Adeno-Associated Virus: Methods and Protocols, pp. 1-23, 2011). Methods for generating and purifying AAV vectors have been described in detail (See e.g., Mueller et al., (2012) CURRENT PROTOCOLS IN MICROBIOLOGY, 14D.1.1-14D.1.21, Production and Discovery of Novel Recombinant Adeno-Associated Viral Vectors). Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, and 8,163,543, U.S. Patent Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the present invention. Similarly, it is contemplated that any adenoviral type may be used, and a person of skill in the art will be able to identify AAV and adenoviral types suitable for the production of their desired recombinant AAV vector (rAAV). AAV particles may be purified, for example by affinity chromatography, iodixonal gradient, or CsCl gradient.

AAV vectors may have single-stranded genomes that are 4.7 kb in size, or are larger or smaller than 4.7 kb, including oversized genomes that are as large as 5.2 kb, or as small as 3.0 kb. Thus, where the exogenous gene of interest to be expressed from the AAV vector is small, the AAV genome may comprise a stuffer sequence. Further, vector genomes may be substantially self-complementary thereby allowing for rapid expression in the cell. In certain embodiments, the genome of a self-complementary AAV vector comprises from 5′ to 3′: a 5′ ITR; a first nucleic acid sequence comprising a promoter and/or enhancer operably linked to a coding sequence of a gene of interest; a modified ITR that does not have a functional terminal resolution site; a second nucleic acid sequence complementary or substantially complementary to the first nucleic acid sequence; and a 3′ ITR. AAV vectors containing genomes of all types are suitable for use in the method of the present invention.

Non-limiting examples of AAV vectors include pAAV-MCS (Agilent Technologies), pAAVK-EF1α-MCS (System Bio Catalog # AAV502A-1), pAAVK-EF1α-MCS1-CMV-MCS2 (System Bio Catalog # AAV503A-1), pAAV-ZsGreen1 (Clontech Catalog #6231), pAAV-MCS2 (Addgene Plasmid #46954), AAV-Stuffer (Addgene Plasmid #106248), pAAVscCBPIGpluc (Addgene Plasmid #35645), AAVS1_Puro_PGK1_3×FLAG_Twin_Strep (Addgene Plasmid #68375), pAAV-RAM-d2TTA::TRE-MCS-WPRE-pA (Addgene Plasmid #63931), pAAV-UbC (Addgene Plasmid #62806), pAAVS1-P-MCS (Addgene Plasmid #80488), pAAV-Gateway (Addgene Plasmid #32671), pAAV-Puro_siKD (Addgene Plasmid #86695), pAAVS1-Nst-MCS (Addgene Plasmid #80487), pAAVS1-Nst-CAG-DEST (Addgene Plasmid #80489), pAAVS1-P-CAG-DEST (Addgene Plasmid #80490), pAAVf-EnhCB-lacZnls (Addgene Plasmid #35642), and pAAVS1-shRNA (Addgene Plasmid #82697). These vectors can be modified to be suitable for therapeutic use. For example, an exogenous gene of interest can be inserted in a multiple cloning site, and a selection marker (e.g., puro or a gene encoding a fluorescent protein) can be deleted or replaced with another (same or different) exogenous gene of interest. Further examples of

AAV vectors are disclosed in U.S. Pat. Nos. 5,871,982, 6,270,996, 7,238,526, 6,943,019, 6,953,690, 9,150,882, and 8,298,818, U.S. Patent Publication No. 2009/0087413, and PCT Publication Nos. WO2017075335A1, WO2017075338A2, and WO2017201258A1.

Lentivirus Vectors

In certain embodiments, the viral vector can be a retroviral vector. Examples of retroviral vectors include moloney murine leukemia virus vectors, spleen necrosis virus vectors, and vectors derived from retroviruses such as rous sarcoma virus, harvey sarcoma virus, avian leukosis virus, human immunodeficiency virus, myeloproliferative sarcoma virus, and mammary tumor virus. Retroviral vectors are useful as agents to mediate retroviral-mediated gene transfer into eukaryotic cells.

In certain embodiments, the retroviral vector is a lentiviral vector. Exemplary lentiviral vectors include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by the gene(s) of interest. Often, the structural genes (i.e., gag, pol, and env), are removed from the retroviral backbone using genetic engineering techniques known in the art. Accordingly, a minimum retroviral vector comprises from 5′ to 3′: a 5′ long terminal repeat (LTR), a packaging signal, an optional exogenous promoter and/or enhancer, an exogenous gene of interest, and a 3′ LTR. If no exogenous promoter is provided, gene expression is driven by the 5′ LTR, which is a weak promoter and requires the presence of Tat to activate expression. The structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

The genes can be incorporated into the proviral backbone in several general ways. The most straightforward constructions are ones in which the structural genes of the retrovirus are replaced by a single gene that is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Accordingly, the new gene(s) are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain embodiments, the R region comprises a trans-activation response (TAR) genetic element, which interacts with the trans-activator (tat) genetic element to enhance viral replication. This element is not required in embodiments wherein the U3 region of the 5′ LTR is replaced by a heterologous promoter.

In certain embodiments, the retroviral vector comprises a modified 5′ LTR and/or 3′ LTR. Modifications of the 3′ LTR are often made to improve the safety of lentiviral or retroviral systems by rendering viruses replication-defective. In specific embodiments, the retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In a further embodiment, the 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also included in the invention.

In certain embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system.

Adjacent the 5′ LTR are sequences necessary for reverse transcription of the genome (the tRNA primer binding site) and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. VIROLOGY, 69(4):2101-09). The packaging signal may be a minimal packaging signal (also referred to as the psi [Ψ] sequence) needed for encapsidation of the viral genome.

In certain embodiments, the retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) CELL, 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In particular embodiments, the retroviral vector backbones comprise one or more FLAP elements upstream or downstream of the heterologous genes of interest in the vectors. For example, in particular embodiments, a transfer plasmid includes a FLAP element. In one embodiment, a vector of the invention comprises a FLAP element isolated from HIV-1.

In certain embodiments, the retroviral vector (e.g., lentiviral vector) further comprises an export element. In one embodiment, retroviral vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. VIROL. 65: 1053; and Cullen et al., (1991) CELL 58: 423) and the hepatitis B virus post-transcriptional regulatory element (HPRE). Generally, the RNA export element is placed within the 3′ UTR of a gene, and can be inserted as one or multiple copies.

In certain embodiments, the retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. VIROL., 73:2886); the posttranscriptional regulatory element present in hepatitis B virus (HPRE) (Huang et al., MOL. CELL. BIOL., 5:3864); and the like (Liu et al., (1995), GENES DEV., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In certain embodiments, vectors of the invention lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain embodiments, vectors of the invention lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in certain embodiments, the retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded. Illustrative examples of polyadenylation signals that can be used in a vector of the invention, includes an ideal polyadenylation sequence (e.g., AATAAA, ATTAAA AGTAAA), a bovine growth hormone polyadenylation sequence (BGHpA), a rabbit β-globin polyadenylation sequence (rβgpA), or another suitable heterologous or endogenous polyadenylation sequence known in the art.

In certain embodiments, a retroviral vector further comprises an insulator element. Insulator elements may contribute to protecting retrovirus-expressed sequences, e.g., therapeutic genes, from integration site effects, which may be mediated by cis-acting elements present in genomic DNA and lead to deregulated expression of transferred sequences (i.e., position effect; see, e.g., Burgess-Beusse et al., (2002) PROC. NATL. ACAD. Sci., USA, 99:16433; and Zhan et al., 2001, HUM. GENET., 109:471). In certain embodiments, the retroviral vector comprises an insulator element in one or both LTRs or elsewhere in the region of the vector that integrates into the cellular genome. Suitable insulators for use in the invention include, but are not limited to, the chicken β-globin insulator (see Chung et al., (1993). CELL 74:505; Chung et al., (1997) PROC. NATL. ACAD. SCI., USA 94:575; and Bell et al., 1999. CELL 98:387). Examples of insulator elements include, but are not limited to, an insulator from a β-globin locus, such as chicken HS4.

Non-limiting examples of lentiviral vectors include pLVX-EF1alpha-AcGFP1-C1 (Clontech Catalog #631984), pLVX-EF1alpha-IRES-mCherry (Clontech Catalog #631987), pLVX-Puro (Clontech Catalog #632159), pLVX-IRES-Puro (Clontech Catalog #632186), pLenti6/V5-DEST™ (Thermo Fisher), pLenti6.2/V5-DEST™ (Thermo Fisher), pLKO.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous gene of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

Adenoviral Vectors

In certain embodiments, the viral vector can be an adenoviral vector. Adenoviruses are medium-sized (90-100 nm), non-enveloped (naked), icosahedral viruses composed of a nucleocapsid and a double-stranded linear DNA genome. The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

A human adenovirus can be used as the source of the adenoviral genome for the adenoviral vector. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serogroup or serotype. Adenoviral serotypes 1 through 51 are available from the American Type Culture Collection (ATCC, Manassas, Va.). Non-group C adenoviral vectors, methods of producing non-group C adenoviral vectors, and methods of using non- group C adenoviral vectors are disclosed in, for example, U.S. Pat. Nos. 5,801,030, 5,837,511, and 5,849,561, and PCT Publication Nos. WO1997/012986 and WO1998/053087.

Non-human adenovirus (e.g., ape, simian, avian, canine, ovine, or bovine adenoviruses) can be used to generate the adenoviral vector (i.e., as a source of the adenoviral genome for the adenoviral vector). For example, the adenoviral vector can be based on a simian adenovirus, including both new world and old world monkeys (see, e.g., Virus Taxonomy: VHIth Report of the International Committee on Taxonomy of Viruses (2005)). A phylogeny analysis of adenoviruses that infect primates is disclosed in, e.g., Roy et al. (2009) PLOS PATHOG. 5(7):e1000503. A gorilla adenovirus can be used as the source of the adenoviral genome for the adenoviral vector. Gorilla adenoviruses and adenoviral vectors are described in, e.g., PCT Publication Nos. WO2013/052799, WO2013/052811, and WO2013/052832. The adenoviral vector can also comprise a combination of subtypes and thereby be a “chimeric” adenoviral vector.

The adenoviral vector can be replication-competent, conditionally replication-competent, or replication-deficient. A replication-competent adenoviral vector can replicate in typical host cells, i.e., cells typically capable of being infected by an adenovirus. A conditionally-replicating adenoviral vector is an adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., a promoter. Conditionally-replicating adenoviral vectors are further described in U.S. Pat. No. 5,998,205. A replication-deficient adenoviral vector is an adenoviral vector that requires complementation of one or more gene functions or regions of the adenoviral genome that are required for replication, as a result of, for example, a deficiency in one or more replication-essential gene function or regions, such that the adenoviral vector does not replicate in typical host cells, especially those in a human to be infected by the adenoviral vector.

Preferably, the adenoviral vector is replication-deficient, such that the replication-deficient adenoviral vector requires complementation of at least one replication-essential gene function of one or more regions of the adenoviral genome for propagation (e.g., to form adenoviral vector particles). The adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad)). See, e.g., Morsy et al. (1998) PROC. NATL. ACAD. SCI. USA 95: 965-976, Chen et al. (1997) PROC. NATL. ACAD. SCI. USA 94: 1645-1650, and Kochanek et al. (1999) HUM. GENE THER. 10(15):2451-9. Examples of replication-deficient adenoviral vectors are disclosed in U.S. Pat. Nos. 5,837,511, 5,851,806, 5,994,106, 6,127,175, 6,482,616, and 7,195,896, and PCT Publication Nos. WO1994/028152, WO1995/002697, WO1995/016772, WO1995/034671, WO1996/022378, WO1997/012986, WO1997/021826, and WO2003/022311.

The replication-deficient adenoviral vector of the invention can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al. (1977) J. GEN. VIROL. 36: 59-72), PER.C6 cells (described in, e.g., PCT Publication No. WO1997/000326, and U.S. Pat. Nos. 5,994,128 and 6,033,908), and 293-ORF6 cells (described in, e.g., PCT Publication No. WO1995/034671 and Brough et al. (1997) J. VIROL. 71: 9206-9213). Other suitable complementing cell lines to produce the replication-deficient adenoviral vector of the invention include complementing cells that have been generated to propagate adenoviral vectors encoding transgenes whose expression inhibits viral growth in host cells (see, e.g., U.S. Patent Publication No. 2008/0233650). Additional suitable complementing cells are described in, for example, U.S. Pat. Nos. 6,677,156 and 6,682,929, and PCT Publication No. WO2003/020879. Formulations for adenoviral vector-containing compositions are further described in, for example, U.S. Pat. Nos. 6,225,289, and 6,514,943, and PCT Publication No. WO2000/034444.

Additional exemplary adenoviral vectors, and/or methods for making or propagating adenoviral vectors are described in U.S. Pat. Nos. 5,559,099, 5,837,511, 5,846,782, 5,851,806, 5,994,106, 5,994,128, 5,965,541, 5,981,225, 6,040,174, 6,020,191, 6,083,716, 6,113,913, 6,303,362, 7,067,310, and 9,073,980.

Commercially available adenoviral vector systems include the ViraPower™ Adenoviral Expression System available from Thermo Fisher Scientific, the AdEasy™ adenoviral vector system available from Agilent Technologies, and the Adeno-X™ Expression System 3 available from Takara Bio USA, Inc.

### V. Host Cells and Cell Lines

Also encompassed by the invention are host cells or cell lines (e.g., prokaryotic or eukaryotic host cells or cell lines) that include a tRNA, aminoacyl-tRNA synthetase, unnatural amino acid, nucleic acid, and/or vector disclosed herein. The nucleic acid encoding the engineered tRNA and aminoacyl-tRNA synthetase can be expressed in an expression host cell either as an autonomously replicating vector within the expression host cell (e.g., a plasmid, or viral particle) or via a stable integrated element or series of stable integrated elements in the genome of the expression host cell, e.g., a mammalian host cell.

Host cells are genetically engineered (including but not limited to, transformed, transduced or transfected), for example, using nucleic acids or vectors disclosed herein. For example, in certain embodiments, one or more vectors include coding regions for an orthogonal tRNA, an orthogonal aminoacyl-tRNA synthetase, and, optionally, a protein to be modified by the inclusion of one or more UAAs, which are operably linked to gene expression control elements that are functional in the desired host cell or cell line. For example, the genes encoding tRNA synthetase and tRNA and an optional selectable marker (e.g., an antibiotic resistance gene, e.g., a puromycin resistance cassette) can be integrated in a transfer vector (e.g., a plasmid, which can be linearized prior to transfection), where for example, the genes encoding the tRNA synthetase can be under the control of a polymerase II promoter (e.g., CMV, EF1α, UbiC, or PGK, e.g., CMV or EF1α) and the genes encoding the tRNA can be under the control of a polymerase III promoter (e.g., U6, 7SK, or H1, e.g., U6). The vectors are transfected into cells and/or microorganisms by standard methods including electroporation or infection by viral vectors, and clones can selected via expression of the selectable marker (for example, by antibiotic resistance).

Exemplary prokaryotic host cells or cell lines include cells derived from a bacteria, e.g., Escherichia coli, Thermus thermophilus, Bacillus stearothermophilus, Pseudomonas fluorescens, Pseudomonas aeruginosa, and Pseudomonas putida. Exemplary eukaryotic host cells or cell lines include cells derived from a plant (e.g., a complex plant such as a monocot or dicot), an algae, a protist, a fungus, a yeast (including Saccharomyces cerevisiae), or an animal (including a mammal, an insect, an arthropod, etc.). Additional exemplary host cells or cell lines include HEK293, HEK293T, Expi293, CHO, CHOK1, Sf9, Sf21, HeLa, U20S, A549, HT1080, CAD, P19, NIH 3T3, L929, N2a, MCF-7, Y79, SO-RB50, HepG2, DUKX-X11, J558L, BHK, COS, Vero, NSO, or ESCs. It is understood that a host cell or cell line can include individual colonies, isolated populations (monoclonal), or a heterogeneous mixture of cells.

A contemplated cell or cell line includes, for example, one or multiple copies of an orthogonal tRNA/aminoacyl-tRNA synthetase pair, optionally stably maintained in the cell's genome or another piece of DNA maintained by the cell. For example, the cell or cell line may contain one or more copies of (i) a tryptophanyl tRNA/aminoacyl-tRNA synthetase pair (wild type or engineered) stably maintained by the cell, and/or (ii) a leucyl tRNA/aminoacyl-tRNA synthetase pair (wild-type or engineered) stably maintained by the cell.

For example, in certain embodiments, the cell line is a stable cell line and the cell line comprises a genome having stably integrated therein (i) a nucleic acid sequence encoding an aminoacyl-tRNA synthetase (e.g., a prokaryotic tryptophanyl-tRNA synthetase mutein capable of charging a tRNA with an unnatural amino acid or a prokaryotic leucyl-tRNA synthetase mutein capable of charging a tRNA with an unnatural amino acid, e.g., a tRNA synthetase mutein disclosed herein); and/or (ii) a nucleic acid sequence encoding a suppressor tRNA (e.g., prokaryotic suppressor tryptophanyl-tRNA capable of being charged with an unnatural amino acid or prokaryotic suppressor leucyl-tRNA capable of being charged with an unnatural amino acid, e.g., a suppressor tRNA disclosed herein).

In certain embodiments, the cell line is capable of expressing the target protein for at least 5, 10, 15, 20, 25, 30, or 35 days (e.g., when the cells are maintained in continuous culture). In certain embodiments, the cell line is capable of expressing the target protein for from 5 to 30 days, 5 to 20 days, 5 to 10 days, 10 to 30 days, 10 to 20 days, or 20 to 30 days (e.g., when the cells are maintained in continuous culture).

In certain embodiments, the cell line is capable of expressing the target protein at a level of expression that is at least 30%, 40%, 50%, 60%, 70%, 80%, or 90%, or about 100% of the level of expression of the template protein expressed in a corresponding cell line from the gene lacking a premature stop codon, for example, the cell line is capable of expressing the target protein at the level of expression for at least 5, 10, 15, 20, 25, 30, or 35 days (e.g., when the cells are maintained in continuous culture).

Methods to introduce a nucleic acid encoding a tRNA and/or an aminoacyl-tRNA synthetase into the genome of a cell of interest, or to stably maintain the nucleic acid in DNA replicated by the cell that is outside of the genome, are well known in the art.

The nucleic acid encoding the tRNA and/or an aminoacyl-tRNA synthetase can be provided to the cell in an expression vector, transfer vector, or DNA cassette, e.g., an expression vector, transfer vector, or DNA cassette disclosed herein. The expression vector transfer vector, or DNA cassette encoding the tRNA and/or aminoacyl-tRNA synthetase can contain one or more copies of the tRNA and/or aminoacyl-tRNA synthetase optionally under the control of an inducible or constitutively active promoter. The expression vector, transfer vector, or DNA cassette may, for example, contain other standard components (enhancers, terminators, etc.). It is contemplated that the nucleic acid encoding the tRNA and the nucleic acid encoding the aminoacyl-tRNA synthetase may be on the same or different vector, may be present in the same or different ratios, and may be introduced into the cell, or stably integrated in the cellular genome, at the same time or sequentially.

One or multiple copies of a DNA cassette encoding the tRNA and/or aminoacyl-tRNA synthetase can be integrated into a host cell genome or stably maintained in the cell using a transposon system (e.g., PiggyBac), a viral vector (e.g., a lentiviral vector or other retroviral vector), CRISPR/Cas9 based recombination, electroporation and natural recombination, a B×B1 recombinase system, or using a replicating/maintained piece of DNA (such as one derived from Epstein-Barr virus).

In order to select for cell lines which stably maintain the nucleic acid encoding the tRNA and/or aminoacyl-tRNA synthetase and/or are efficient at incorporating UAAs into a protein of interest, a selectable marker can be used. Exemplary selectable markers include zeocin, puromycin, neomycin, dihydrofolate reductase (DHFR), glutamine synthetase (GS), mCherry-EGFP fusion, or other fluorescent proteins. In certain embodiments, a gene encoding a selectable marker protein (or a gene encoding a protein required for a detectable function, e.g., viability, in the presence of the selectable marker) may include a premature stop codon, such that the protein will only be expressed if the cell line is capable of incorporating a UAA at the site of the premature stop codon.

In certain embodiments, to develop a host cell or cell line including two or more tRNA/aminoacyl-tRNA synthetase pairs, one can use multiple identical or distinct UAA directing codons in order to identify host cells or cell lines which have incorporated multiple copies of the two or more tRNA/aminoacyl-tRNA synthetase pairs through iterative rounds of genomic integration and selection. Host cells or cell lines which contain enhanced UAA incorporation efficiency, low background, and decreased toxicity can first be isolated via a selectable marker containing one or more stop codons. Subsequently, the host cells or cell lines can be subjected to a selection scheme to identify host cells or cell lines which contain the desired copies of tRNA/aminoacyl-tRNA synthetase pairs and express a gene of interest (either genomically integrated or not) containing one or more stop codons. Protein expression may be assayed using any method known in the art, including for example, Western blot using an antibody that binds the protein of interest or a C-terminal tag.

The host cells or cell lines be cultured in conventional nutrient media modified as appropriate for such activities as, for example, screening steps, activating promoters or selecting transformants. These cells can optionally be cultured into transgenic organisms. Other useful references, e.g. for cell isolation and culture (e.g., for subsequent nucleic acid isolation) include Freshney (1994) Culture of Animal Cells, a Manual of Basic Technique, third edition, Wiley-Liss, New York; Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg N.Y.) and Atlas and Parks (eds.) The Handbook of Microbiological Media (1993) CRC Press, Boca Raton, Fla.

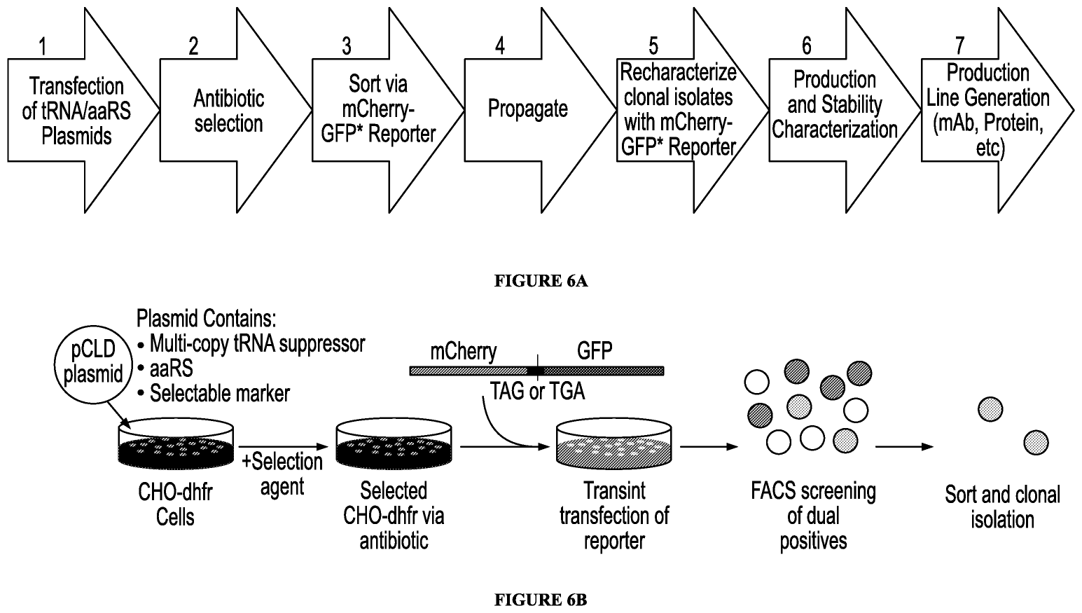

The production of an exemplary cell line capable of producing antibodies incorporating a UAA is described in Roy et al. (2020) MABS 12(1), e1684749). Additional exemplary methods to generate stable cell lines for the incorporation of a UAA into a protein are described in Example 2 and FIG. 6 herein.

In certain embodiments, a method for the generation of a stable cell line for the incorporation of a UAA into a protein comprises one or more of the following steps: (i) transfecting cells with one or more plasmids encoding a suppressor tRNA and an aminoacyl-tRNA synthetase, wherein the one or more plasmids include a selectable marker (e.g., an antibiotic resistance gene) (ii) selecting cells that contain the one or more plasmids using the selectable marker, (iii) transiently transfecting cells with a reporter construct (e.g., a fluorescent reporter construct) that gives a detectable signal upon UAA incorporation into a protein, (iv) selecting cells that are capable of UAA incorporation using the reporter construct, and (v) further propagating the cells. In certain embodiments, the method further comprises (vi) transiently transfecting cells with the reporter construct again, and selecting cells that have maintained capability of UAA incorporation using the reporter construct.

### VI. Proteins Including Unnatural Amino Acids (UAAs) and Methods of Making the Same

Also encompassed by the invention are proteins including unnatural amino acids (UAAs) and methods of making the same.

The incorporation of an unnatural amino acid can be done for a variety of purposes, including tailoring changes in protein structure and/or function, changing size, acidity, nucleophilicity, hydrogen bonding, hydrophobicity, accessibility of protease target sites, targeting to a moiety (e.g., for a protein array), adding a biologically active molecule, attaching a polymer, attaching a radionuclide, modulating serum half-life, modulating tissue penetration (e.g. tumors), modulating active transport, modulating tissue, cell or organ specificity or distribution, modulating immunogenicity, modulating protease resistance, etc. Proteins that include an unnatural amino acid can have enhanced or even entirely new catalytic or biophysical properties. For example, the following properties are optionally modified by inclusion of an unnatural amino acid into a protein: toxicity, biodistribution, structural properties, spectroscopic properties, chemical and/or photochemical properties, catalytic ability, half-life (including but not limited to, serum half-life), ability to react with other molecules, including but not limited to, covalently or noncovalently, and the like. The compositions including proteins that include at least one unnatural amino acid are useful for, including but not limited to, novel therapeutics, diagnostics, enzymes, and binding proteins (e.g., therapeutic antibodies).

A protein may have at least one, for example, at least two, at least three, at least four, at least five, at least six, at least seven, at least eight, at least nine, or at least ten or more UAAs. The UAAs can be the same or different. For example, there can be 1, 2, 3, 4, 5, 6, 7, 8, 9, or 10 or more different sites in the protein that comprise 1, 2, 3, 4, 5, 6, 7, 8, 9, or 10 or more different UAAs. A protein may have at least one, but fewer than all, of a particular amino acid present in the protein substituted with the UAA. For a given protein with more than one UAA, the UAA can be identical or different (for example, the protein can include two or more different types of UAAs, or can include two of the same UAA). For a given protein with more than two UAAs, the UAAs can be the same, different or a combination of a multiple unnatural amino acid of the same kind with at least one different UAA.

In certain embodiments, the protein is an antibody (or a fragment thereof), bispecific antibody, nanobody, affibody, viral protein, chemokine, antigen, blood coagulation factor, hormone, growth factor, enzyme, or any other polypeptide or protein.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

Additional examples of therapeutic, diagnostic, and other proteins that can be modified to comprise one or more unnatural amino acids are described in U.S. Patent Application Publication Nos. 2003/0082575 and 2005/0009049.

tRNAS, aminoacyl-tRNA synthetases, and/or unnatural amino acids disclosed herein may be used to incorporate an unnatural amino acid into a protein of interest using any appropriate translation system.

The term “translation system” refers to a system including components necessary to incorporate an amino acid into a growing polypeptide chain (protein). Components of a translation system can include, e.g., ribosomes, tRNA's, synthetases, mRNA and the like. Translation systems may be cellular or cell-free, and may be prokaryotic or eukaryotic. For example, translation systems may include, or be derived from, a non-eukaryotic cell, e.g., a bacterium (such as E. coli), a eukaryotic cell, e.g., a yeast cell, a mammalian cell, a plant cell, an algae cell, a fungus cell, or an insect cell.

Translation systems include host cells or cell lines, e.g., host cells or cell lines contemplated herein. To express a polypeptide of interest with an unnatural amino acid in a host cell, one may clone a polynucleotide encoding the polypeptide into an expression vector that contains, for example, a promoter to direct transcription, a transcription/translation terminator, and if for a nucleic acid encoding a protein, a ribosome binding site for translational initiation.

Translation systems also include whole cell preparations such as permeabilized cells or cell cultures wherein a desired nucleic acid sequence can be transcribed to mRNA and the mRNA translated. Cell-free translation systems are commercially available and many different types and systems are well-known. Examples of cell-free systems include, but are not limited to, prokaryotic lysates such as Escherichia coli lysates, and eukaryotic lysates such as wheat germ extracts, insect cell lysates, rabbit reticulocyte lysates, rabbit oocyte lysates and human cell lysates. Reconstituted translation systems may also be used. Reconstituted translation systems may include mixtures of purified translation factors as well as combinations of lysates or lysates supplemented with purified translation factors such as initiation factor-1 (IF-1), IF-2, IF-3 (α or β), elongation factor T (EF-Tu), or termination factors. Cell-free systems may also be coupled transcription/translation systems wherein DNA is introduced to the system, transcribed into mRNA and the mRNA is translated.

The invention provides methods of expressing a protein containing an unnatural amino acid and methods of producing a protein with one, or more, unnatural amino acids at specified positions in the protein. The methods comprise incubating a translation system (e.g., culturing or growing a host cell or cell line, e.g., a host cell or cell line disclosed herein) under conditions that permit incorporation of the unnatural amino acid into the protein being expressed in the cell. The translation system may be contacted with (e.g. the cell culture medium may be contacted with) one, or more, unnatural amino acids (e.g., leucyl or tryptophanyl analogs) under conditions suitable for incorporation of the one, or more, unnatural amino acids into the protein.

In certain embodiments, the protein is expressed from a nucleic acid sequence comprising a premature stop codon. The translation system (e.g., host cell or cell line) may, for example, contain a leucyl-tRNA synthetase mutein (e.g., a leucyl-tRNA synthetase mutein disclosed herein) capable of charging a suppressor leucyl tRNA (e.g., a suppressor leucyl tRNA disclosed herein) with an unnatural amino acid (e.g., a leucyl analog) which is incorporated into the protein at a position corresponding to the premature stop codon. In certain embodiments, the leucyl suppressor tRNA comprises an anticodon sequence that hybridizes to the premature stop codon and permits the unnatural amino to be incorporated into the protein at the position corresponding to the premature stop codon.

In certain embodiments, the protein is expressed from a nucleic acid sequence comprising a premature stop codon. The translation system (e.g., host cell or cell line) may, for example, contain a tryptophanyl-tRNA synthetase mutein (e.g., a tryptophanyl-tRNA synthetase mutein disclosed herein) capable of charging a suppressor tryptophanyl tRNA (e.g., a suppressor tryptophanyl tRNA disclosed herein) with an unnatural amino acid (e.g., a tryptophan analog) which is incorporated into the protein at a position corresponding to the premature stop codon. In certain embodiments, the tryptophanyl suppressor tRNA comprises an anticodon sequence that hybridizes to the premature stop codon and permits the unnatural amino to be incorporated into the protein at the position corresponding to the premature stop codon.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

SEQUENCE LISTING

| SEQ | | |
|---|---|---|
| ID | Descrip- | |
| NO | tion | Sequence |
| 1 | E. coli | MQEQYRPEEIESKVQLHWDEKRTFEVTEDESKEKYYCLSMLPYPSGR |
| | LeuRS | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDTFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWYYARYTCPQYKEGMLDSEA |
| | | ANYWLPVDIYIGGIEHAIMHLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 1) |
| 2 | LeuRSN1 | MEEQYRPEEIESKVQLHWDKKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVA |
| | | AGHPLAQKAAENNPELAAFIDECRNTKVAEAEMATMEKKGVDTGFKA |
| | | VHPLTGEEIPVWAANFVLMEYGTGAVMAVPGHDQRDYEFASKYGLNI |
| | | KPVILAADGSEPDLSQQALTEKGVLFNSGEFNGLDHEAAFNAIADKL |
| | | TAMGVGERKVNYRLRDWGVSRQRYWGAPIPMVTLEDGTVMPTPDDQL |
| | | PVILPEDVVMDGITSPIKADPEWAKTTVNGMPALRETDTFDTFMESS |
| | | WIYARYTCPQYKEGMLDSEAANYWLPVDIAIGGIEHAIMGLLYFRFF |
| | | HKLMRDAGMVNSDEPAKQLLCQGMVLADAFYYVGENGERNWVSPVDA |
| | | IVERDEKGRIVKAKDAAGHELVYTGMSKMSKSKNNGIDPQVMVERYG |
| | | ADTVRLFMMFASPADMTLEWQESGVEGANRFLKRVWKLVYEHTAKGD |
| | | VAALNVDALTENQKALRRDVHKTIAKVTDDIGRRQTFNTAIAAIMEL |
| | | MNKLAKAPTDGEQDRALMQEALLAVVRMLNPFTPHICFTLWQELKGE |
| | | GDIDNAPWPVADEKAMVEDSTLVVVQVNGKVRAKITVPVDATEEQVR |
| | | ERAGQEHLVAKYLDGVTVRKVIYVPGKLLNLVVGGPV |
| | | (SEQ ID NO: 2) |
| 3 | LeuRSN2 | MEEQYRPEEIESKVQLHWDKKRTFEVTEDESKEKYYCLSVSPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDRFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWSYARYTCPQYKEGMLDSEA |
| | | ANYWLPVDILIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 3) |
| 4 | LeuRSN3 | MEEQYRPEEIESKVQLHWDEKRTFEVTEDESKEKYYCLSILPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 4) |
| 5 | V9 | MEEQYRPEEIESKVQLHWDMKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 5) |
| 6 | V10 | MEEQYRPEEIESKVQLHWDVKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 6) |
| 7 | V26 | MEEQYRPEEIESKVQLHWDKKRTFEVTEDESKEKYYCLSIVPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 7) |
| 8 | V29 | MEEQYRPEEIESKVQLHWDKKRTFEVTEDESKEKYYCLSIAPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIAIGGIEHAIMGLLYFRFFHKLMRDAGMVNSDEPAKQLL |
| | | CQGMVLADAFYYVGEN |
| | | GERNWVSPVDAIVERDEKGRIVKAKDAAGHELVYTGMSKMSKSKNNG |
| | | IDPQVMVERYGADTVRLFMMFASPADMTLEWQESGVEGANRELKRVW |
| | | KLVYEHTAKGDVAALNVDALTENQKALRRDVHKTIAKVTDDIGRRQT |
| | | FNTAIAAIMELMNKLAKAPTDGEQDRALMQEALLAVVRMLNPFTPHI |
| | | CFTLWQELKGEGDIDNAPWPVADEKAMVEDSTLVVVQVNGKVRAKIT |
| | | VPVDATEEQVRERAGQEHLVAKYLDGVTVRK |
| | | VIYVPGKLLNLVVGGPV |
| | | (SEQ ID NO: 8) |
| 9 | V48 | MEEQYRPEEIESKVQLHWDkKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWAYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFKRVWKLVYEHTAKGDVAALNVDALTENQKALRRDVH |
| | | KTIAKVTDDIGRRQTFNTAIAAIMELMNKLAKAPTDGEQDRALMQEA |
| | | LLAVVRMLNPFTPHICFTLWQELKGEGDIDNAPWPVADEKAMVEDST |
| | | LVVVQVNGKVRAKITVPVDATEEQVRERAGQEHLVAKYLDGVTVRKV |
| | | IYVPGKLLNLVVGGPV |
| | | (SEQ ID NO: 9) |
| 10 | V50 | MEEQYRPEEIESKVQLHWDkKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWHYARYTCPQYKEGMLDSEA |
| | | ANYWLPVDIAIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 10) |
| 11 | V54 | MEEQYRPEEIESKVQLHWDkKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIIIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 11) |
| 12 | V55 | MEEQYRPEEIESKVQLHWDKKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIVIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 12) |
| 13 | V56 | MEEQYRPEEIESKVQLHWDKKRTFEVTEDESKEKYYCLSISPYPSGR |
| | | LHMGHVRNYTIGDVIARYQRMLGKNVLQPIGWDAFGLPAEGAAVKNN |
| | | TAPAPWTYDNIAYMKNQLKMLGFGYDWSRELATCTPEYYRWEQKFFT |
| | | ELYKKGLVYKKTSAVNWCPNDQTVLANEQVIDGCCWRCDTKVERKEI |
| | | PQWFIKITAYADELLNDLDKLDHWPDTVKTMQRNWIGRSEGVEITFN |
| | | VNDYDNTLTVYTTRPDAFMGCTYLAVAAGHPLAQKAAENNPELAAFI |
| | | DECRNTKVAEAEMATMEKKGVDTGFKAVHPLTGEEIPVWAANFVLME |
| | | YGTGAVMAVPGHDQRDYEFASKYGLNIKPVILAADGSEPDLSQQALT |
| | | EKGVLFNSGEFNGLDHEAAFNAIADKLTAMGVGERKVNYRLRDWGVS |
| | | RQRYWGAPIPMVTLEDGTVMPTPDDQLPVILPEDVVMDGITSPIKAD |
| | | PEWAKTTVNGMPALRETDTFDTFMESSWIYARYTCPQYKEGMLDSEA |
| | | ANYWLPVDIGIGGIEHAIMGLLYFRFFHKLMRDAGMVNSDEPAKQLL |
| | | CQGMVLADAFYYVGENGERNWVSPVDAIVERDEKGRIVKAKDAAGHE |
| | | LVYTGMSKMSKSKNNGIDPQVMVERYGADTVRLFMMFASPADMTLEW |
| | | QESGVEGANRFLKRVWKLVYEHTAKGDVAALNVDALTENQKALRRDV |
| | | HKTIAKVTDDIGRRQTFNTAIAAIMELMNKLAKAPTDGEQDRALMQE |
| | | ALLAVVRMLNPFTPHICFTLWQELKGEGDIDNAPWPVADEKAMVEDS |
| | | TLVVVQVNGKVRAKITVPVDATEEQVRERAGQEHLVAKYLDGVTVRK |
| | | VIYVPGKLLNLVVGGPV |
| | | (SEQ ID NO: 13) |
| 14 | | MX₂EQYRPEEIESKVQLHWDX₂₀KRTFEVTEDESKEKYYCLSX₄₀X₄₁P |
| | | YPSGRLHMGHVRNYTIGDVIARYQRMLGKNVLQPIGWDAFGLPAEGA |
| | | AVKNNTAPAPWTYDNIAYMKNQLKMLGFGYDWSRELATCTPEYYRWE |
| | | QKFFTELYKKGLVYKKTSAVNWCPNDQTVLANEQVIDGCCWRCDTKV |
| | | ERKEIPQWFIKITAYADELLNDLDKLDHWPDTVKTMQRNWIGRSEGV |
| | | ElTFNVNDYDNTLTVYTTRPDX₂₅₂FMGCTYLAVAAGHPLAQKAAENN |
| | | PELAAFIDECRNTKVAEAEMATMEKKGVDTGFKAVHPLTGEEIPVWA |
| | | ANFVLMEYGTGAVMAVPGHDQRDYEFASKYGLNIKPVILAADGSEPD |
| | | LSQQALTEKGVLFNSGEFNGLDHEAAFNAIADKLTAMGVGERKVNYR |
| | | LRDWGVSRQRYWGAPIPMVTLEDGTVMPTPDDQLPVILPEDVVMDGI |
| | | TSPIKADPEWAKTTVNGMPALRETDTFDTFMESSWX₄₉₉YARYTCPQY |
| | | KEGMLDSEAANYWLPVDIX₅₂₇IGGIEHAIMX₅₃₇LLYFRFFHKLMRDA |
| | | GMVNSDEPAKQLLCQGMVLADAFYYVGENGERNWVSPVDAIVERDEK |
| | | GRIVKAKDAAGHELVYTGMSKMSKSKNNGIDPQVMVERYGADTVRLF |
| | | MMFASPADMTLEWQESGVEGANRFLKRVWKLVYEHTAKGDVAALNVD |
| | | ALTENQKALRRDVHKTIAKVTDDIGRRQTFNTAIAAIMELMNKLAKA |
| | | PTDGEQDRALMQEALLAVVRMLNPFTPHICFTLWQELKGEGDIDNAP |
| | | WPVADEKAMVEDSTLVVVQVNGKVRAKITVPVDATEEQVRERAGQEH |
| | | LVAKYLDGVTVRKVIYVPGKLLNLVVGGPV |
| | | wherein X₂ is Q or E, X₂₀ is E, K, V or M, X₄₀ is |
| | | M, I, or V, X₄₁ is L, S, V, or A, X₂₅₂ is T, A, |
| | | or R, X₄₉₉ is Y, A, I, H, or S, X₅₂₇ is Y, A, I, |
| | | L, or V, and X₅₃₇ is H or G |
| | | (SEQ ID NO: 14) |
| 15 | | MEEQYRPEEIESKVQLHWDX₂₀KRTFEVTEDESKEKYYCLSIX₄₁PYP |
| | | SGRLHMGHVRNYTIGDVIARYQRMLGKNVLQPIGWDAFGLPAEGAAV |
| | | KNNTAPAPWTYDNIAYMKNQLKMLGFGYDWSRELATCTPEYYRWEQK |
| | | FFTELYKKGLVYKKTSAVNWCPNDQTVLANEQVIDGCCWRCDTKVER |
| | | KEIPQWFIKITAYADELLNDLDKLDHWPDTVKTMQRNWIGRSEGVEI |
| | | TFNVNDYDNTLTVYTTRPDAFMGCTYLAVAAGHPLAQKAAENNPELA |
| | | AFIDECRNTKVAEAEMATMEKKGVDTGFKAVHPLTGEEIPVWAANFV |
| | | LMEYGTGAVMAVPGHDQRDYEFASKYGLNIKPVILAADGSEPDLSQQ |
| | | ALTEKGVLFNSGEFNGLDHEAAFNAIADKLTAMGVGERKVNYRLRDW |
| | | GVSRQRYWGAPIPMVTLEDGTVMPTPDDQLPVILPEDVVMDGITSPI |
| | | KADPEWAKTTVNGMPALRETDTFDTEMESSWX₄₉₉YARYTCPQYKEGM |
| | | LDSEAANYWLPVDIX₅₂₇IGGIEHAIMGLLYFRFFHKLMRDAGMVNSD |
| | | EPAKQLLCQGMVLADAFYYVGENGERNWVSPVDAIVERDEKGRIVKA |
| | | KDAAGHELVYTGMSKMSKSKNNGIDPQVMVERYGADTVRLFMMFASP |
| | | ADMTLEWQESGVEGANRFLKRVWKLVYEHTAKGDVAALNVDALTENQ |
| | | KALRRDVHKTIAKVTDDIGRRQTFNTAIAAIMELMNKLAKAPTDGEQ |
| | | DRALMQEALLAVVRMLNPFTPHICFTLWQELKGEGDIDNAPWPVADE |
| | | KAMVEDSTLVVVQVNGKVRAKITVPVDATEEQVRERAGQEHLVAKYL |
| | | DGVTVRKVIYVPGKLLNLVVGGPV |
| | | wherein X₂₀ is K, V or M, X₄₁ is S, V, or A, X₄₉₉ |
| | | is A, I, or H, and X₅₂₇ is A, I, or V |
| | | (SEQ ID NO: 15) |
| 16 | Lcu- | GCCCGGATGGTGGAATCGGTAGACACAAGGGATTctaAATCCCTCGG |
| | tRNA.wtC | CGTTCGCGCTGTGCGGGTTCAAGTCCCGCTCCGGGtA |
| | UA | (SEQ ID NO: 16) |
| 17 | Leu- | GCCCGGATGGTGGAATCGGTAGACACAAGGGATTtcaAATCCCTCGG |
| | tRNA.wtU | CGTTCGCGCTGTGCGGGTTCAAGTCCCGCTCCGGGtA |
| | CA | (SEQ ID NO: 17) |
| 18 | Lcu- | GCCCGGATGGTGGAATCGGTAGACACAAGGGATTttaAATCCCTCGG |
| | tRNA.wtU | CGTTCGCGCTGTGCGGGTTCAAGTCCCGCTCCGGGtA |
| | UA | (SEQ ID NO: 18) |
| 19 | Lcu- | GCCCGGATGGTGGAATCGGTAGACACAAGGGACTctaAATCCCTCGG |
| | tRNA.h1C | CGTTCGCGCTGTGCGGGTTCAAGTCCCGCTCCGGGcA |
| | UA | (SEQ ID NO: 19) |
| 20 | Leu- | GCCCGGATGGTGGAATCGGTAGACACAAGGGACTtcaAATCCCTCGG |
| | tRNA.h1U | CGTTCGCGCTGTGCGGGTTCAAGTCCCGCTCCGGGcA |
| | CA | (SEQ ID NO: 20) |
| 21 | Lcu- | GCCCGGATGGTGGAATCGGTAGACACAAGGGACTttaAATCCCTCGG |
| | tRNA.h1U | CGTTCGCGCTGTGCGGGTTCAAGTCCCGCTCCGGGcA |
| | UA | (SEQ ID NO: 21) |
| 22 | Lcu- | GCCCGGATGGTGGAATCGGTAGACACAAGGGATTctaAATCCCTCGG |
| | tRNA.wtC | CGTTCGCGCTGTGCGGGTTCAAGTCCCGCTCCGGGtACCA |
| | UA cca | (SEQ ID NO: 22) |
| 23 | Leu- | GCCCGGATGGTGGAATCGGTAGACACAAGGGATTtcaAATCCCTCGG |
| | tRNA.wtU | CGTTCGCGCTGTGCGGGTTCAAGTCCCGCTCCGGGtACCA |
| | CA cca | (SEQ ID NO: 23) |
| 24 | Lcu- | GCCCGGATGGTGGAATCGGTAGACACAAGGGATTttaAATCCCTCGG |
| | tRNA.wtU | CGTTCGCGCTGTGCGGGTTCAAGTCCCGCTCCGGGtACCA |
| | UA cca | (SEQ ID NO: 24) |
| 25 | Lcu- | GCCCGGATGGTGGAATCGGTAGACACAAGGGACTctaAATCCCTCGG |
| | tRNA.h1C | CGTTCGCGCTGTGCGGGTTCAAGTCCCGCTCCGGGcACCA |
| | UA cca | (SEQ ID NO: 25) |
| 26 | Leu- | GCCCGGATGGTGGAATCGGTAGACACAAGGGACTtcaAATCCCTCGG |
| | tRNA.h1U | CGTTCGCGCTGTGCGGGTTCAAGTCCCGCTCCGGGcACCA |
| | CA cca | (SEQ ID NO: 26) |
| 27 | Lcu- | GCCCGGATGGTGGAATCGGTAGACACAAGGGACTttaAATCCCTCGG |
| | tRNA.h1U | CGTTCGCGCTGTGCGGGTTCAAGTCCCGCTCCGGGcACCA |
| | UA cca | (SEQ ID NO: 27) |
| 28 | Leu- | GCCCGGATGGTGGAATCGGTAGACACAAGGGACTnnnAATCCCTCGG |
| | tRNA.003. | CGTTCGCGCTGTGCGGGTTCAAGTCCCGCTCCGGGCACCA |
| | 30 | wherein nnn is CTA, TCA, or TTA |
| | | (SEQ ID NO: 27) |
| 29 | Lcu- | GGGCGTGTGGTGGAATCGGTAGACACAAGGGATTnnnAATCCCTCGG |
| | tRNA.003. | CGTTCGCGCTGTGCGGGTTCAAGTCCCGCCGCGCCCACCA |
| | 31 | wherein nnn is CTA, TCA, or TTA |
| | | (SEQ ID NO: 28) |
| 30 | Lcu- | GGGCGCGTGGTGGAATCGGTAGACACAAGGGATTnnnAATCCCTCGG |
| | tRNA.003. | CGTTCGCGCTGTGCGGGTTCAAGTCCCGCCGCGCCCACCA |
| | 32 | wherein nnn is CTA, TCA, or TTA |
| | | (SEQ ID NO: 29) |
| 31 | Leu- | GGGCATGTGGTGGAATCGGTAGACACAAGGGATTnnnAATCCCTCGG |
| | tRNA.003. | CGTTCGCGCTGTGCGGGTTCAAGTCCCGCCATGCCCACCA |
| | 33 | wherein nnn is CTA, TCA, or TTA |
| | | (SEQ ID NO: 30) |
| 32 | Lcu- | GGGCACGTGGTGGAATCGGTAGACACAAGGGATTnnnAATCCCTCGG |
| | tRNA.003. | CGTTCGCGCTGTGCGGGTTCAAGTCCCGCCGTGCCCACCA |
| | 34 | wherein nnn is CTA, TCA, or TTA |
| | | (SEQ ID NO: 31) |
| 33 | Leu- | GGGGGTGTGGTGGAATCGGTAGACACAAGGGATTnnnAATCCCTCGG |
| | tRNA.003. | CGTTCGCGCTGTGCGGGTTCAAGTCCCGCCGCCCCCACCA |
| | 35 | wherein nnn is CTA, TCA, or TTA |
| | | (SEQ ID NO: 32) |
| 34 | Leu- | GGGGGCGTGGTGGAATCGGTAGACACAAGGGATTnnnAATCCCTCGG |
| | t RNA.003. | CGTTCGCGCTGTGCGGGTTCAAGTCCCGCCGTCCCCACCA |
| | 36 | wherein nnn is CTA, TCA, or TTA |
| | | (SEQ ID NO: 33) |
| 35 | Lcu- | GGGGATGTGGTGGAATCGGTAGACACAAGGGATTnnnAATCCCTCGG |
| | tRNA.003. | CGTTCGCGCTGTGCGGGTTCAAGTCCCGCCGTCCCCACCA |
| | 37 | wherein nnn is CTA, TCA, or TTA |
| | | (SEQ ID NO: 34) |
| 36 | Leu- | GGGGACGTGGTGGAATCGGTAGACACAAGGGATTnnnAATCCCTCGG |
| | tRNA.003. | CGTTCGCGCTGTGCGGGTTCAAGTCCCGCCGTCCCCACCA |
| | 38 | wherein nnn is CTA, TCA, or TTA |
| | | (SEQ ID NO: 35) |
| 37 | Leu- | GCCCGTATGGTGGAATCGGTAGACACAAGGGATTnnnAATCCCTCGG |
| | tRNA.003. | CGTTCGCGCTGTGCGGGTTCAAGTCCCGCTGCGGGCACCA |
| | 39 | wherein nnn is CTA, TCA, or TTA |
| | | (SEQ ID NO: 36) |
| 38 | Lcu- | GGGATAGTGGTGGAATCGGTAGACACAAGGGATTnnnAATCCCTCGG |
| | tRNA.003. | CGTTCGCGCTGTGCGGGTTCAAGTCCCGCCTATCCCACCA |
| | 40 | wherein nnn is CTA, TCA, or TTA |
| | | (SEQ ID NO: 37) |
| 39 | Lcu- | GGGCATGTGGTGGAATCGGTAGACACAAGGGATTnnnAATCCCTCGG |
| | tRNA.003. | CGTTCGCGCTGTGCGGGTTCAAGTCCCGCCGTGCCCACCA |
| | 41 | wherein nnn is CTA, TCA, or TTA |
| | | (SEQ ID NO: 38) |
| 40 | Leu- | GGGCAGATGGTGGAATCGGTAGACACAAGGGATTnnnAATCCCTCGG |
| | tRNA.003. | CGTTCGCGCTGTGCGGGTTCAAGTCCCGCTCTGCCCACCA |
| | 42 | wherein nnn is CTA, TCA, or TTA |
| | | (SEQ ID NO: 39) |
| 41 | Lcu- | GGGCGTATGGTGGAATCGGTAGACACAAGGGATTnnnAATCCCTCGG |
| | tRNA.003. | CGTTCGCGCTGTGCGGGTTCAAGTCCCGCTGCGCCCACCA |
| | 43 | wherein nnn is CTA, TCA, or TTA |
| | | (SEQ ID NO: 40) |
| | Leu- | GGGCAAGTGGTGGAATCGGTAGACACAAGGGATTnnnAATCCCTCGG |
| | tRNA.003. | CGTTCGCGCTGTGCGGGTTCAAGTCCCGCCTTGCCCACCA |
| | 44 | wherein nnn is CTA, TCA, or TTA |
| | | (SEQ ID NO: 41) |
| 42 | Leu- | GCACACATGGTGGAATCGGTAGACACAAGGGATTnnnAATCCCTCGG |
| | tRNA.003. | CGTTCGCGCTGTGCGGGTTCAAGTCCCGCTGTGTGCACCA |
| | 45 | wherein nnn is CTA, TCA, or TTA |
| | | (SEQ ID NO: 42) |
| 43 | E. coli | MTKPIVFSGAQPSGELTIGNYMGALRQWVNMQDDYHCIYCIVDQHAI |
| | TrpRS.wt | TVRQDAQKLRKATLDTLALYLACGIDPEKSTIFVQSHVPEHAQLGWA |
| | | LNCYTYFGELSRMTQFKDKSARYAENINAGLFDYPVLMAADILLYQT |
| | | NLVPVGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| | | (SEQ ID NO: 43) |
| 44 | TrpRS.h14 | MTKPIVFAGAQPSGELTIGNYMGALRQWVNMQDDYHCIYCIVDQHAI |
| | | TVRQDAQKLRKATLDTLALYLACGIDPEKSTIFVQSHVPEHAQLGWA |
| | | LNCYTYFGELSRMTQFKDKSARYAENINAGLFDYPVLMAADILLYQT |
| | | NLGPCGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| | | (SEQ ID NO: 44) |
| 45 | TrpRS.h13 | MTKPIVFAGAQPSGELTIGNYMGALRQWVNMQDDYHCIYCIVDQHAI |
| | | TVRQDAQKLRKATLDTLALYLACGIDPEKSTIFVQSHVPEHAQLGWA |
| | | LNCYTYFGELSRMTQFKDKSARYAENINAGLFDYPVLMAADILLYQT |
| | | NLAPAGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| | | (SEQ ID NO: 45) |
| 46 | TrpRS.h9 | MTKPIVFAGAQPSGELTIGNYMGALRQWVNMQDDYHCIYCIVDQHAI |
| | | TVRQDAQKLRKATLDTLALYLACGIDPEKSTIFVQSHVPEHAQLGWA |
| | | LNCYTYFGELSRMTQFKDKSARYAENINAGLFDYPVLMAADILLYQT |
| | | NLSPAGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| | | (SEQ ID NO: 46) |
| 474 | TrpRS.h10 | MTKPIVFAGAQPSGELTIGNYMGALRQWVNMQDDYHCIYCIVDQHAI |
| | | TVRQDAQKLRKATLDTLALYLACGIDPEKSTIFVQSHVPEHAQLGWA |
| | | LNCYTYFGELSRMTQFKDKSARYAENINAGLFDYPVLMAADILLYQT |
| | | NLGPAGEDQKQHLELSRDIAQRFNALYGEIFKVPEPFIPKSGARVMS |
| | | LLEPTKKMSKSDDNRNNVIGLLEDPKSVVKKIKRAVTDSDEPPVVRY |
| | | DVQNKAGVSNLLDILSAVTGQSIPELEKQFEGKMYGHLKGEVADAVS |
| | | GMLTELQERYHRFRNDEAFLQQVMKDGAEKASAHASRTLKAVYEAIG |
| | | FVAKP |
| | | (SEQ ID NO: 47) |
| 48 | E. coli | AGGGGCGTAGTTCAATTGGTAGAGCACCGGTCTccaAAACCGGGTGT |
| | Trp- | TGGGAGTTCGAGTCTCTCCGCCCCTGCCA |
| | tRNA | (SEQ ID NO: 48) |
| 49 | Trp- | AGGGGCGTAGTTCAATTGGTAGAGCACCGGTCTctaAAACCGGGTGT |
| | tRNACUA | TGGGAGTTCGAGTCTCTCCGCCCCTGCCA |
| | | (SEQ ID NO: 49) |
| 50 | Trp- | AGGGGCGTAGTTCAATTGGTAGAGCACCGGTCTtcaAAACCGGGTGT |
| | tRNAUCA | TGGGAGTTCGAGTCTCTCCGCCCCTGCCA |
| | | (SEQ ID NO: 50) |
| 51 | Trp- | AGGGGCGTAGTTCAATTGGTAGAGCACCGGTCTttaAAACCGGGTGT |
| | tRNAUUA | TGGGAGTTCGAGTCTCTCCGCCCCTGCCA |
| | | (SEQ ID NO: 51) |
| 52 | Trp- | TGGGGTATCGCCAAGCGGTAAGGCACCGGATTCnnnTTCCGGCATTC |
| | tRNADOL | CGAGGTTCGAATCCTCGTACCCCAGCCA |
| | 4 | wherein nnn is CTA, TCA, or TTA |
| | | (SEQ ID NO: 52) |
| 53 | Trp- | AGGGGCATAGCTCAAGCGGTAAAGCACCGGACTnnnAAACCGGCAGT |
| | tRNADOL | CCGAAGTTCGAATCCCCCCACCCCAGCCA |
| | 5 | wherein nnn is CTA, TCA, or TTA |
| | | (SEQ ID NO: 53) |
| 54 | E. coli | ATGCAGGAACAGTATCGCCCGGAAGAAATTGAAAGCAAAGTGCAGCT |
| LeuRS | | GCATTGGGATGAAAAACGCACCTTTGAAGTGACCGAAGATGAAAGCA |
| | | AAGAAAAATATTATTGCCTGAGCATGCTGCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TACCTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGTATTATGCGC |
| | | GCTATACCTGCCCGCAGTATAAAGAAGGCATGCTGGATAGCGAAGCG |
| | | GCGAACTATTGGCTGCCGGTGGATATTTATATTGGCGGCATTGAACA |
| | | TGCGATTATGCATCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 54) |
| 55 | LeuRS. | ATGGAAGAACAGTATCGCCCGGAAGAAATTGAAAGCAAAGTGCAGCT |
| | v1 | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 55) |
| 56 | LeuRS. | ATGGAAGAACAGTATCGCCCGGAAGAAATTGAAAGCAAAGTGCAGCT |
| | v2 | GCATTGGGATAAAAAACGCACCTTTGAAGTGACCGAAGATGAAAGCA |
| | | AAGAAAAATATTATTGCCTGAGCGTGAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TCGCTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGAGCTATGCGC |
| | | GCTATACCTGCCCGCAGTATAAAGAAGGCATGCTGGATAGCGAAGCG |
| | | GCGAACTATTGGCTGCCGGTGGATATTCTGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 56) |
| 57 | LeuRS. | ATGGAAGAACAGTATCGCCCGGAAGAAATTGAAAGCAAAGTGCAGCT |
| | v3 | GCATTGGGATGAAAAACGCACCTTTGAAGTGACCGAAGATGAAAGCA |
| | | AAGAAAAATATTATTGCCTGAGCATTCTGCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 57) |
| 58 | V9 | ATGGAAGAACAGTATCGCCCGGAAGAAATTGAAAGCAAAGTGCAGCT |
| | | GCATTGGGATATGAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 58) |
| 59 | V10 | ATGGAAGAACAGTATCGCCCGGAAGAAATTGAAAGCAAAGTGCAGCT |
| | | GCATTGGGATGTGAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 59) |
| 60 | V26 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTGTGCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 60) |
| 61 | V29 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTGCGCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 61) |
| 62 | V48 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGGCGTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTAAACGCGTGTGGAA |
| | | ACTGGTGTATGAACATACCGCGAAAGGCGATGTGGCGGCGCTGAACG |
| | | TGGATGCGCTGACCGAAAACCAGAAAGCGCTGCGCCGCGATGTGCAT |
| | | AAAACCATTGCGAAAGTGACCGATGATATTGGCCGCCGCCAGACCTT |
| | | TAACACCGCGATTGCGGCGATTATGGAACTGATGAACAAACTGGCGA |
| | | AAGCGCCGACCGATGGCGAACAGGATCGCGCGCTGATGCAGGAAGCG |
| | | CTGCTGGCGGTGGTGCGCATGCTGAACCCGTTTACCCCGCATATTTG |
| | | CTTTACCCTGTGGCAGGAACTGAAAGGCGAAGGCGATATTGATAACG |
| | | CGCCGTGGCCGGTGGCGGATGAAAAAGCGATGGTGGAAGATAGCACC |
| | | CTGGTGGTGGTGCAGGTGAACGGCAAAGTGCGCGCGAAAATTACCGT |
| | | GCCGGTGGATGCGACCGAAGAACAGGTGCGCGAACGCGCGGGCCAGG |
| | | AACATCTGGTGGCGAAATATCTGGATGGCGTGACCGTGCGCAAAGTG |
| | | ATTTATGTGCCGGGCAAACTGCTGAACCTGGTGGTGGGCGGCCCGGT |
| | | G |
| | | (SEQ ID NO: 62) |
| 63 | V50 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGCATTATGCGC |
| | | GCTATACCTGCCCGCAGTATAAAGAAGGCATGCTGGATAGCGAAGCG |
| | | GCGAACTATTGGCTGCCGGTGGATATTGCGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 63) |
| 64 | V54 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTATTATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 64) |
| 65 | V55 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGTGATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 65) |
| 66 | V56 | ATGGAAGAACAGTATCGCCCGGAAGAAATTGAAAGCAAAGTGCAGCT |
| | | GCATTGGGATAAAAAACGCACCTTTGAAGTGACCGAAGATGAAAGCA |
| | | AAGAAAAATATTATTGCCTGAGCATTAGCCCGTATCCGAGCGGCCGC |
| | | CTGCATATGGGCCATGTGCGCAACTATACCATTGGCGATGTGATTGC |
| | | GCGCTATCAGCGCATGCTGGGCAAAAACGTGCTGCAGCCGATTGGCT |
| | | GGGATGCGTTTGGCCTGCCGGCGGAAGGCGCGGCGGTGAAAAACAAC |
| | | ACCGCGCCGGCGCCGTGGACCTATGATAACATTGCGTATATGAAAAA |
| | | CCAGCTGAAAATGCTGGGCTTTGGCTATGATTGGAGCCGCGAACTGG |
| | | CGACCTGCACCCCGGAATATTATCGCTGGGAACAGAAATTTTTTACC |
| | | GAACTGTATAAAAAAGGCCTGGTGTATAAAAAAACCAGCGCGGTGAA |
| | | CTGGTGCCCGAACGATCAGACCGTGCTGGCGAACGAACAGGTGATTG |
| | | ATGGCTGCTGCTGGCGCTGCGATACCAAAGTGGAACGCAAAGAAATT |
| | | CCGCAGTGGTTTATTAAAATTACCGCGTATGCGGATGAACTGCTGAA |
| | | CGATCTGGATAAACTGGATCATTGGCCGGATACCGTGAAAACCATGC |
| | | AGCGCAACTGGATTGGCCGCAGCGAAGGCGTGGAAATTACCTTTAAC |
| | | GTGAACGATTATGATAACACCCTGACCGTGTATACCACCCGCCCGGA |
| | | TGCGTTTATGGGCTGCACCTATCTGGCGGTGGCGGCGGGCCATCCGC |
| | | TGGCGCAGAAAGCGGCGGAAAACAACCCGGAACTGGCGGCGTTTATT |
| | | GATGAATGCCGCAACACCAAAGTGGCGGAAGCGGAAATGGCGACCAT |
| | | GGAAAAAAAAGGCGTGGATACCGGCTTTAAAGCGGTGCATCCGCTGA |
| | | CCGGCGAAGAAATTCCGGTGTGGGCGGCGAACTTTGTGCTGATGGAA |
| | | TATGGCACCGGCGCGGTGATGGCGGTGCCGGGCCATGATCAGCGCGA |
| | | TTATGAATTTGCGAGCAAATATGGCCTGAACATTAAACCGGTGATTC |
| | | TGGCGGCGGATGGCAGCGAACCGGATCTGAGCCAGCAGGCGCTGACC |
| | | GAAAAAGGCGTGCTGTTTAACAGCGGCGAATTTAACGGCCTGGATCA |
| | | TGAAGCGGCGTTTAACGCGATTGCGGATAAACTGACCGCGATGGGCG |
| | | TGGGCGAACGCAAAGTGAACTATCGCCTGCGCGATTGGGGCGTGAGC |
| | | CGCCAGCGCTATTGGGGCGCGCCGATTCCGATGGTGACCCTGGAAGA |
| | | TGGCACCGTGATGCCGACCCCGGATGATCAGCTGCCGGTGATTCTGC |
| | | CGGAAGATGTGGTGATGGATGGCATTACCAGCCCGATTAAAGCGGAT |
| | | CCGGAATGGGCGAAAACCACCGTGAACGGCATGCCGGCGCTGCGCGA |
| | | AACCGATACCTTTGATACCTTTATGGAAAGCAGCTGGATTTATGCGC |
| | | GCTATACCTGCCCGCAGTATAAAGAAGGCATGCTGGATAGCGAAGCG |
| | | GCGAACTATTGGCTGCCGGTGGATATTGGCATTGGCGGCATTGAACA |
| | | TGCGATTATGGGCCTGCTGTATTTTCGCTTTTTTCATAAACTGATGC |
| | | GCGATGCGGGCATGGTGAACAGCGATGAACCGGCGAAACAGCTGCTG |
| | | TGCCAGGGCATGGTGCTGGCGGATGCGTTTTATTATGTGGGCGAAAA |
| | | CGGCGAACGCAACTGGGTGAGCCCGGTGGATGCGATTGTGGAACGCG |
| | | ATGAAAAAGGCCGCATTGTGAAAGCGAAAGATGCGGCGGGCCATGAA |
| | | CTGGTGTATACCGGCATGAGCAAAATGAGCAAAAGCAAAAACAACGG |
| | | CATTGATCCGCAGGTGATGGTGGAACGCTATGGCGCGGATACCGTGC |
| | | GCCTGTTTATGATGTTTGCGAGCCCGGCGGATATGACCCTGGAATGG |
| | | CAGGAAAGCGGCGTGGAAGGCGCGAACCGCTTTCTGAAACGCGTGTG |
| | | GAAACTGGTGTATGAACATACCGCGAAAGGCGATGTGGCGGCGCTGA |
| | | ACGTGGATGCGCTGACCGAAAACCAGAAAGCGCTGCGCCGCGATGTG |
| | | CATAAAACCATTGCGAAAGTGACCGATGATATTGGCCGCCGCCAGAC |
| | | CTTTAACACCGCGATTGCGGCGATTATGGAACTGATGAACAAACTGG |
| | | CGAAAGCGCCGACCGATGGCGAACAGGATCGCGCGCTGATGCAGGAA |
| | | GCGCTGCTGGCGGTGGTGCGCATGCTGAACCCGTTTACCCCGCATAT |
| | | TTGCTTTACCCTGTGGCAGGAACTGAAAGGCGAAGGCGATATTGATA |
| | | ACGCGCCGTGGCCGGTGGCGGATGAAAAAGCGATGGTGGAAGATAGC |
| | | ACCCTGGTGGTGGTGCAGGTGAACGGCAAAGTGCGCGCGAAAATTAC |
| | | CGTGCCGGTGGATGCGACCGAAGAACAGGTGCGCGAACGCGCGGGCC |
| | | AGGAACATCTGGTGGCGAAATATCTGGATGGCGTGACCGTGCGCAAA |
| | | GTGATTTATGTGCCGGGCAAACTGCTGAACCTGGTGGTGGGCGGCCC |
| | | GGTG |
| | | (SEQ ID NO: 66) |

### EXAMPLES

The following Examples are merely illustrative and are not intended to limit the scope or content of the invention in any way.

Example 1—Construction and Selection of Improved Leucyl-tRNA Synthetase Muteins

This example describes the construction of leucyl tRNA-synthetase muteins.

Wild-type E. coli leucyl tRNA-synthetase (SEQ ID NO:1) was cloned into a plasmid under control of a CMV promoter. The plasmid also contained a 4× U6-LeutRNA_{CUA} DNA cassette encoding a suppressor tRNA (E. coli leucyl tRNA h1 with a CUA anticodon, SEQ ID NO: 19). The plasmid, encoding the leucyl-trRNA synthetase and leucyl suppressor tRNA was used as a library template construct and is referred to as pBBK-LeuRS.wt-LtR-TAG.

Leucyl synthetase muteins V2 (SEQ ID NO:3) and V3 (SEQ ID NO:4) were generated via standard mutagenesis of wild-type active site residues (see, Meng et al, (2018) supra). Leucyl synthetase mutein V1 (SEQ ID NO:2) was designed by combining distinct active site mutations of the V2 and V3 muteins.

The plasmid encoding leucyl tRNA-synthetase mutein V1 (SEQ ID NO:2; referred to herein as LeuRS.v1) was then used as a template for the generation of a library of plasmids encoding additional leucyl tRNA-synthetases variants. The library included plasmids encoding leucyl tRNA-synthetase variants with individual substitutions of each of Q2, E20, M40, L41, T252, Y499, Y527, and H537 with each of the twenty natural amino acids.

A sGFP-39TAG reporter fluorescence assay, which utilizes a reporter plasmid encoding the GFP protein with an amber codon at Y39 and fused to a His-tag at the C-terminal (GFP39-TAG), was used to assess the leucyl synthetase mutein activity in mammalian cells. HEK293T cells were cultured in Dulbecco's modified Eagle's Medium (DMEM) supplemented with 10% FBS and 0.5× penicillin-streptomycin in a humidified incubator at 37° C. in the presence of 8% CO₂. 0.7×10⁶ HEK293T cells were seeded per well 24 hours prior to transfection in a 12 well plate. Polyethylamine and DNA were mixed at a ratio of 4 μL PEI (1 mg/mL) to 1 μg DNA in DMEM. For each transfection mixture, 500 ng GFP39-TAG reporter plasmid was mixed with pBBK-LeuRS.v#-LtR-TAG. Unnatural amino acids (UAAs) were added or excluded from the media at concentrations of 0.5 mM LCA, 1 mM LKET, or 1 mM ACA. Fluorescence images were obtained at 48 hours with an Olympus microscope through a 488 bandpass filter set. To obtain GFP39-TAG expression data, cells were harvested at 12,000×g. Cells were washed with PBS and lysed with CelLytic M lysis buffer supplemented with 1× Halt protease inhibitor and 0.01% Pierce universal nuclease. After a 20 minute incubation, the lysate was clarified at 15,000×g for 10 minutes and transferred to a clear bottom 96-well assay plate. Fluorescence was measured using a Fluoroskan Ascent II (Ex. 485 nm; Em. 535 nm).

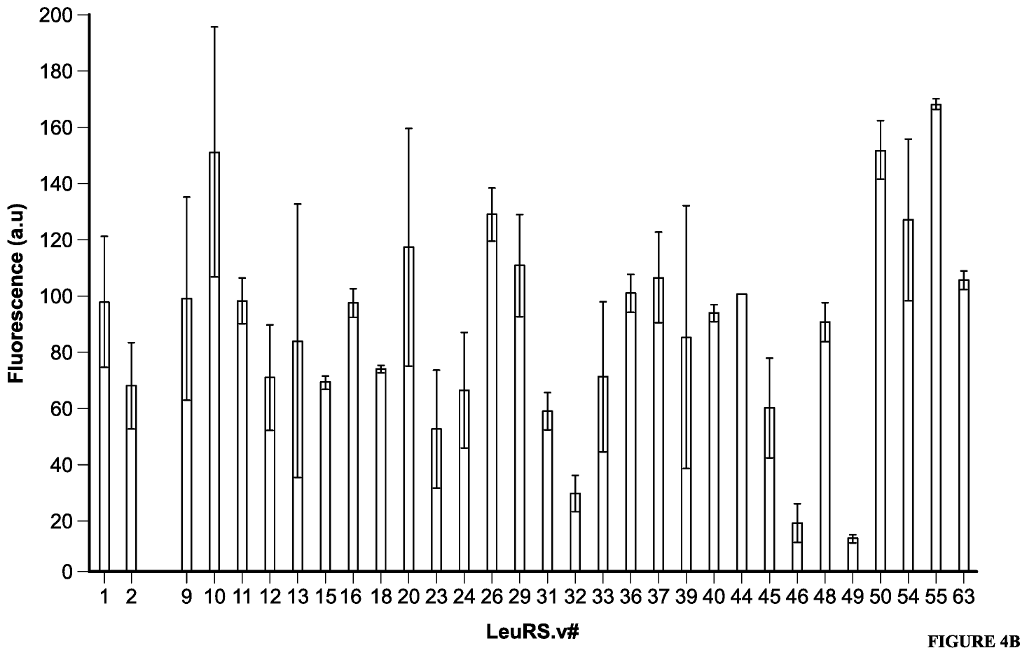

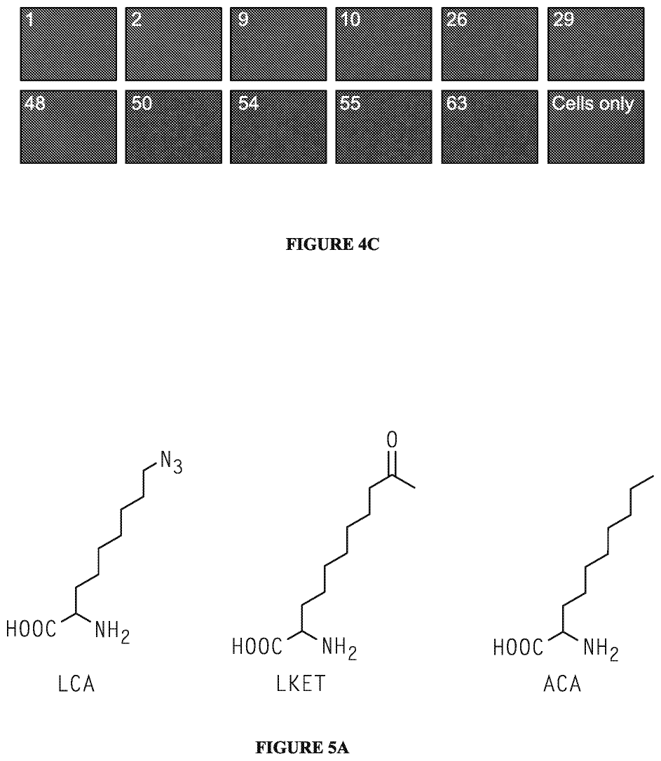

Variants of LeuRS.v1 (SEQ ID NOs: 5-14) were assayed for LCA incorporation as described above. Point mutations in variants of LeuRS.v1 are shown in FIG. 4A, and corresponding fluorescent activity in the GFP-39TAG reporter assay is shown in FIG. 4B. As seen in FIG. 4B, the leucyl tRNA-synthetases that were empirically selected from the high-throughput screen screened had varying activity compared to parent LeuRS.v1 in the presence of 0.5 mM LCA. Over 50 variants were tested; representative fluorescent images for select LeuRS variants (LeuRS variants v1, v2, v9, v10, v26, v29, v48, v50, v54, v55, and v63, with amino acid sequences and mutations as shown in TABLE 1 below) in the GFP-39TAG reporter assay are shown in FIG. 4C. Variants v9, v10, v26, v29, v48, v50, v54, v55, and v63 were found to have enhanced activity for LCA incorporation when compared to the parent sequence, LeuRS.v1.

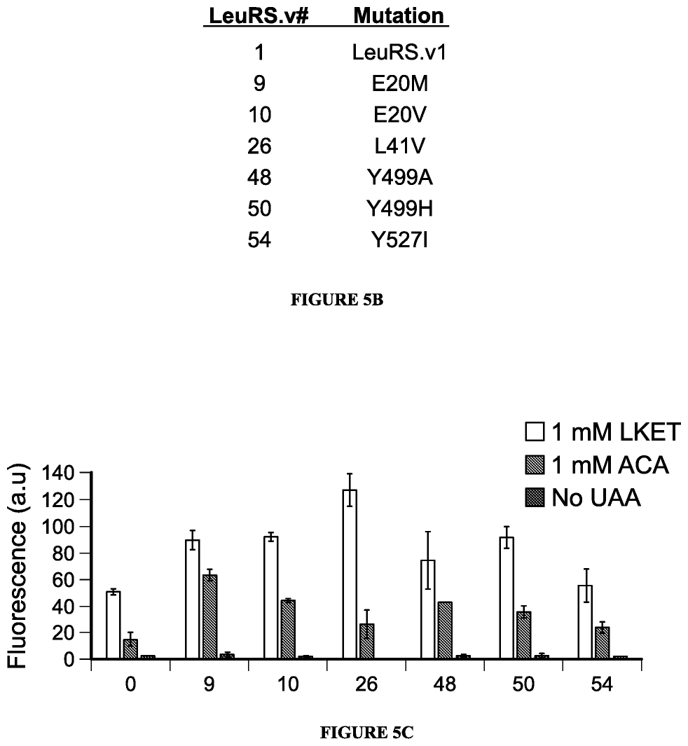

The LeuRS variants depicted in FIGS. 4B-4C were subjected to polyspecificity analysis to test whether they would accept UAA substrates in addition to LCA. GFP-39TAG expression analysis was performed as described above in the presence of 0.5 mM LCA, 1 mM LKET, or 1 mM ACA (FIG. 5A). Activity was measured as described above, and results are depicted in FIG. 5B.

Leucyl synthetase muteins described in this Example with enhanced activity to wild-type are summarized in TABLE 1.

TABLE 1

| LeuRS | Q2 | E20 | M40 | L41 | T252 | Y499 | Y527 | H537 |
|---|---|---|---|---|---|---|---|---|
| WT (SEQ ID NO: 1) | Q | E | M | L | T | Y | Y | H |
| V1 (SEQ ID NO: 2) | E | K | I | S | A | I | A | G |
| V2 (SEQ ID NO: 3) | E | K | V | S | R | S | L | G |
| V3 (SEQ ID NO: 4) | E | E | I | L | A | I | A | G |
| V9 (SEQ ID NO: 5) | E | M | I | S | A | I | A | G |
| V10 (SEQ ID NO: 6) | E | V | I | S | A | I | A | G |
| V26 (SEQ ID NO: 7) | E | K | I | V | A | I | A | G |
| V29 (SEQ ID NO: 8) | E | K | I | A | A | I | A | G |
| V48 (SEQ ID NO: 9) | E | K | I | S | A | A | A | G |
| V50 (SEQ ID NO: 10) | E | K | I | S | A | H | A | G |
| V54 (SEQ ID NO: 11) | E | K | I | S | A | I | I | G |
| V55 (SEQ ID NO: 12) | E | K | I | S | A | I | V | G |
| V63 (SEQ ID NO: 13) | E | K | I | S | A | I | G | G |

### Example 2—Construction of Stable Cell Lines Expressing Leucyl tRNAs and Leucyl tRNA-Synthetase Muteins

This example describes the construction of cells lines, e.g., stable cell lines, expressing leucyl suppressor tRNAs and leucyl tRNA-synthetases (schematically depicted in

FIGS. 6A-6B).

CHO-dhFr adherent cells were acquired from ATCC. CHO-dhFr cells were chosen as a parental cell line due to the flexibility of their inherent metabolic dhFr selection strategy for future integration of target genes of interest (i.e., post-platform cell line generation). CHO-dhFr cells were maintained according to the ATCC protocol in Gibco™ IMDM, supplemented with 10% Fetal Bovine Serum, 0.1 mM hypoxanthine, 0.016 mM thymidine, and 0.002 mM Methotrexate. For stable cell line generation, CHO-dhFr cells under passage 15 were cultured for pCLD plasmid transfection either using Lipofectamine 2000 (Thermo Fisher Scientific) or Nucleofector 4D-X unit and associated kits (Lonza).

For Lipofectamine 2000 transfections, 2 mL of 2.5×10⁵ per mL CHO-dhFr cells were plated per well of a 6 well plate 24 hours prior to transfection. 24 hours later, cells were transfected with 3 μg of a plasmid containing (i) a 4×U6 promoter, leucyl suppressor tRNA h1 repeat cassette and (ii) an EF1α promoter, LeuRS.v1-IRES-puromycin cassette (plasmid 2, TABLE 2) using Lipofectamine 2000 following the Thermo Fisher Scientific standard protocol.

For Lonza Nucleofector 4D-X transfections, 1×10⁶ CHO-dhFr cells transfected with 2-4 μg of plasmid containing (i) a 4×U6 promoter, leucyl suppressor tRNA repeat cassette and (ii) an EF1α promoter, LeuRS.v1-IRES-puromycin cassette (plasmid 2, TABLE 2) using the Lonza kit SE according to the manufacturer's instructions. Cells post-transfection were plated into a 6 well plate. After Lipofectamine 2000-based transfection or Nucleofection, plates were incubated at 37° C. with 5% CO₂ to recover for 24-48 hours prior to application of 1.5 μg-6 μg puromycin for selection (steps 1-2 of FIG. 6A).

The leucyl suppressor tRNA in plasmid 2 is depicted in SEQ ID NO: 19 and the LeuRS.v1 leucyl-tRNA synthetase in plasmid 2 is depicted in SEQ ID NO: 2.

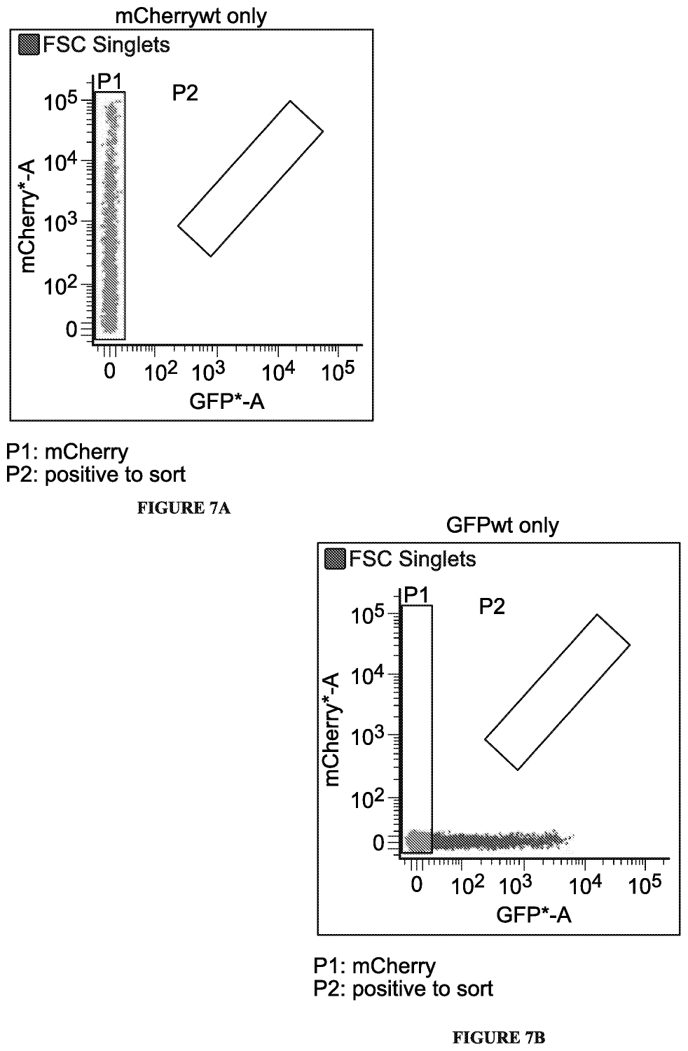

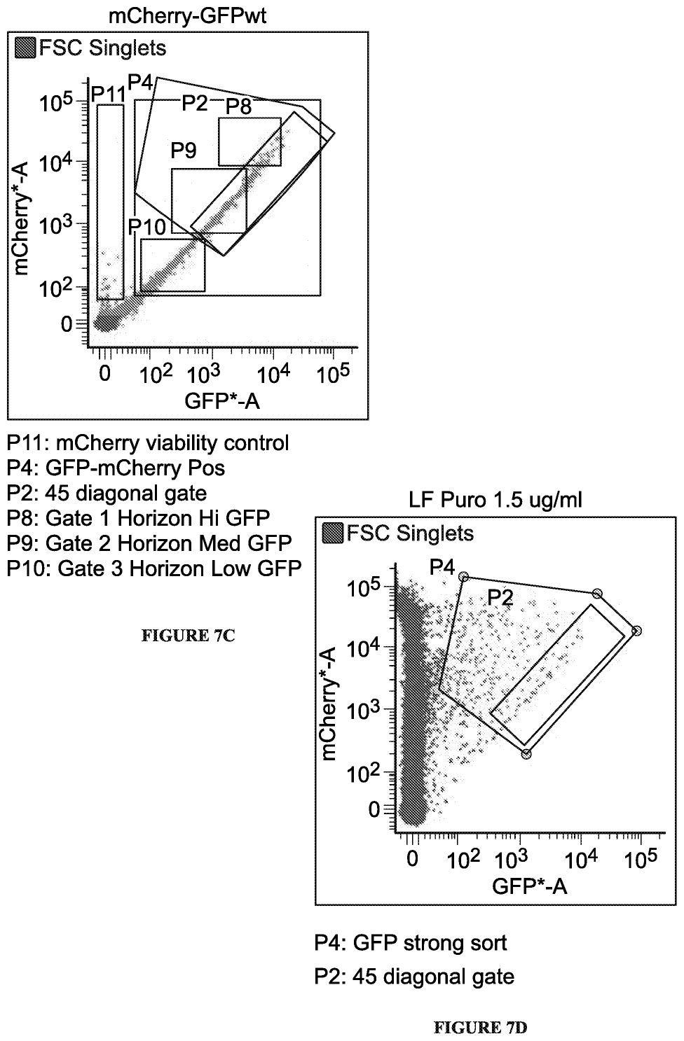

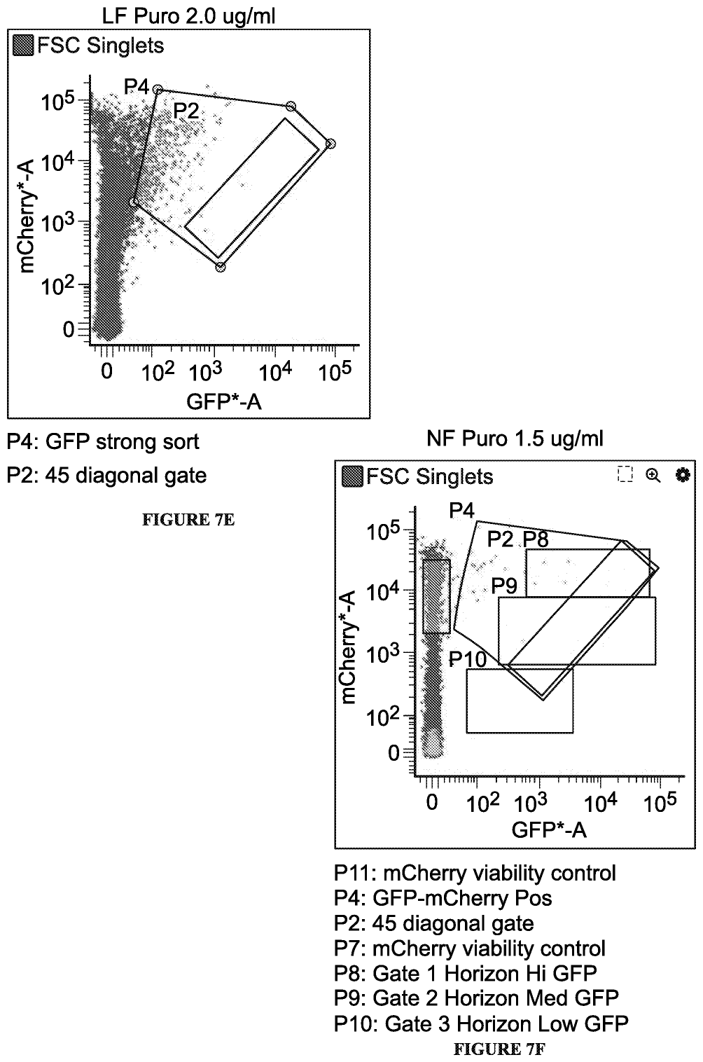

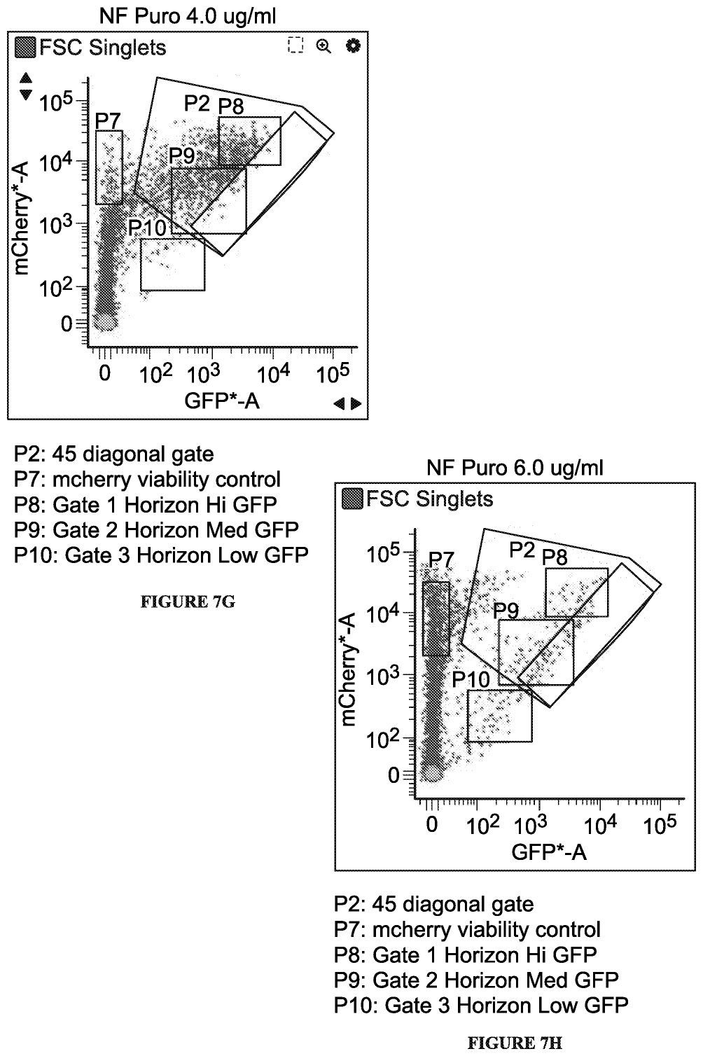

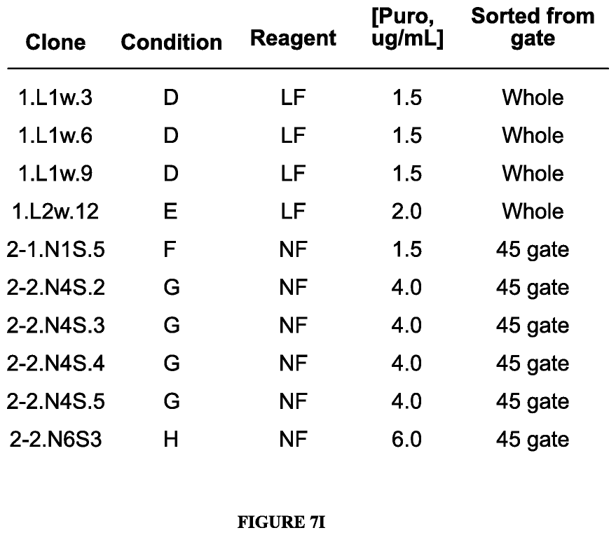

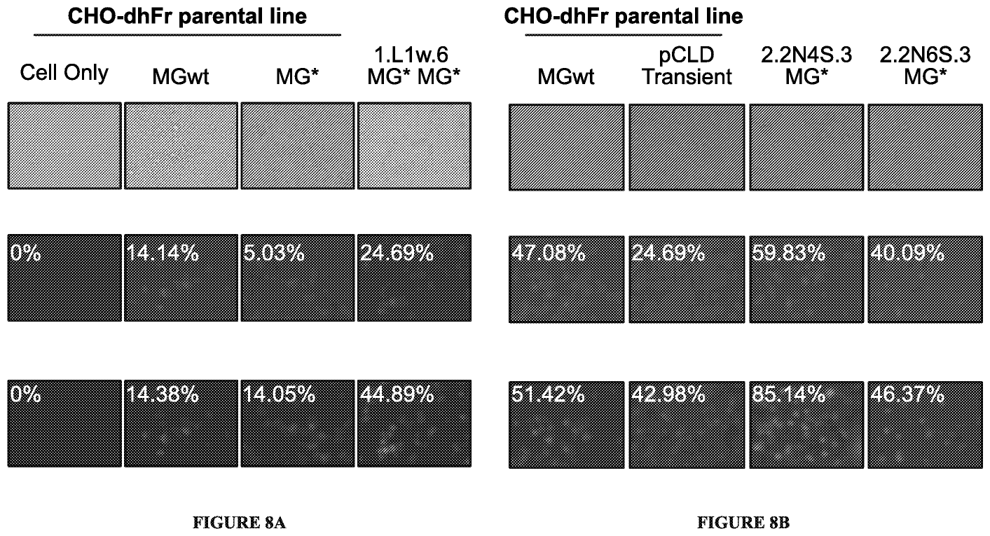

Two weeks post-puromycin selection, the cells were transfected with a dual reporter construct comprising both GFP and mCherry fluorescent reporters using Lipofectamine 2000, as described in the manufacturer's protocol. Said dual reporter constitutively produces mCherry (red) and is connected via a linker comprising a stop codon to GFP, such that the reporter conditionally produces GFP (green) if the tRNA/aaRS pair are active (step 3 of FIG. 6A). Transfected cells were cultured in the presence of 0.25 mM LCA for 15-48 hours before aseptic single cell sorting on a BD Melody FACS sorter (BD). UAA incorporation activity was determined by fluorescence activated cell sorting (FACS) and fluorescent microscopy (FIGS. 7-8). FIGS. 7A-7C demonstrate the expected phenotypes of fluorescent reporter positive controls, demonstrating mCherry-GFP wildtype results in cells populated along the 45 degree axis. FIGS. 7D-7E represent stable cell populations obtained using lipofectamine-based transfection that were selected with puromycin as described above, while FIGS. 7F-7H represent stable cell populations obtained using nucleofection-based transfection that were selected with puromycin as described above. As shown in FIGS. 7A-7H, a population shift into the 45 degree axis, indicative of changes in the conditional GFP signal, demonstrated that nucleofector transfections have the greatest shift, and accordingly, the greatest activation of the conditional dual reporter. In summary, each dot along the 45 degree axis is a stable cell line and can be selected for isolation, propagation, and recharacterization (FIG. 7). During the FACS pool analysis, mCherry and GFP dual positive clones were selected and single clones were sorted into 96 well plates via the “whole” gate, which is the broadest selection depicted in FIG. 7, or the 45 gate along the 45 degree axis (FIG. 7I). These clones were cultured for recovery prior to recharacterization via the conditional dual reporter (step 4 of FIG. 6A).

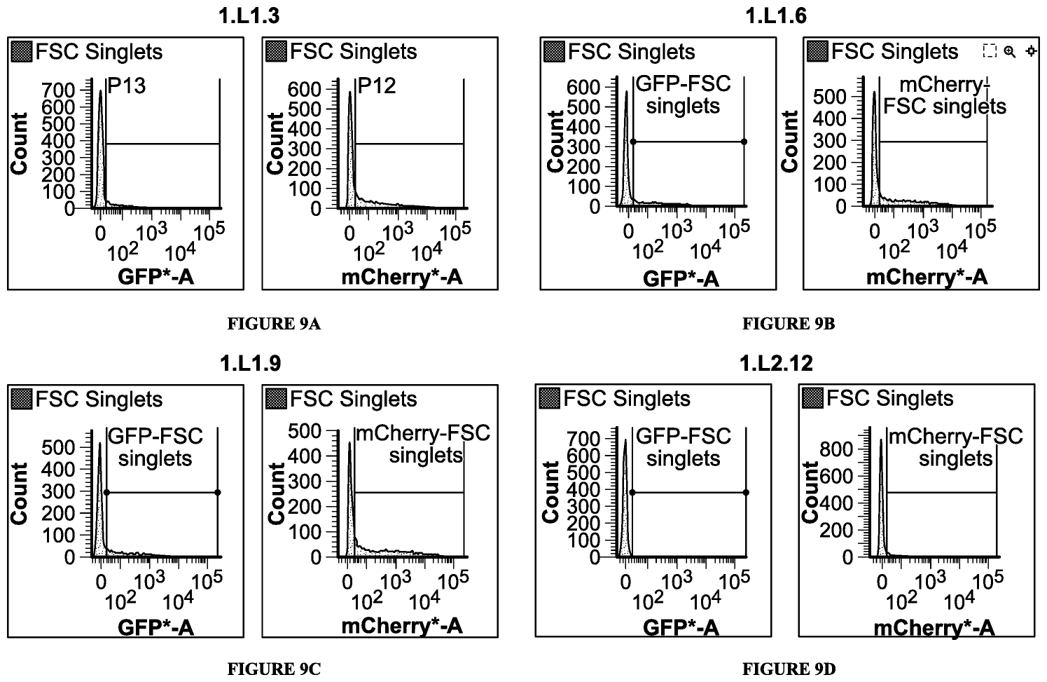

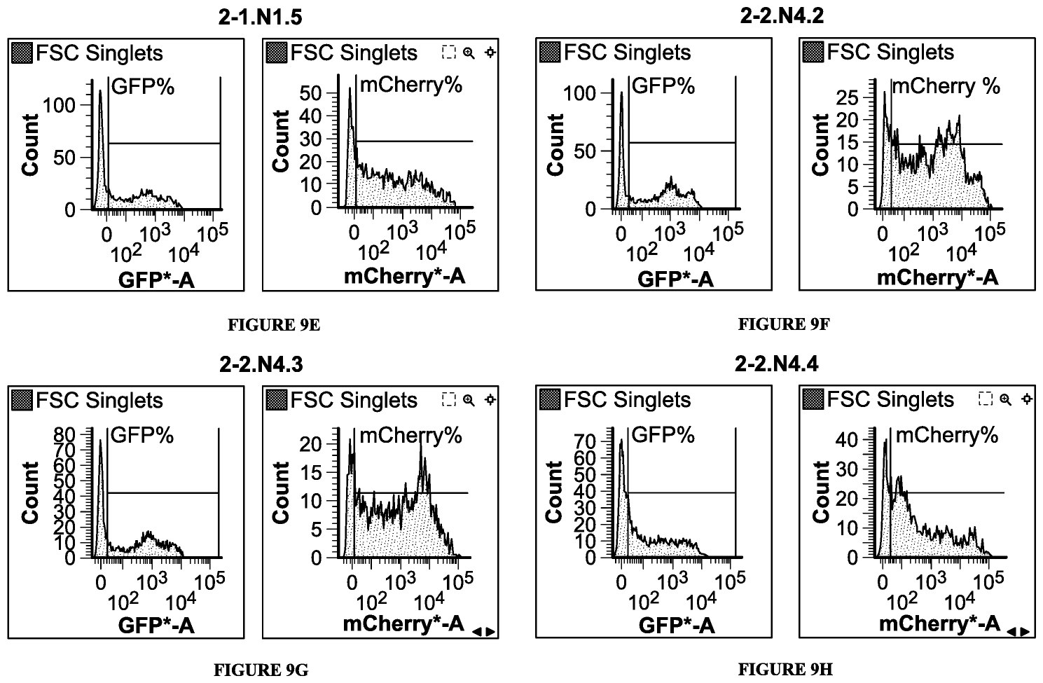

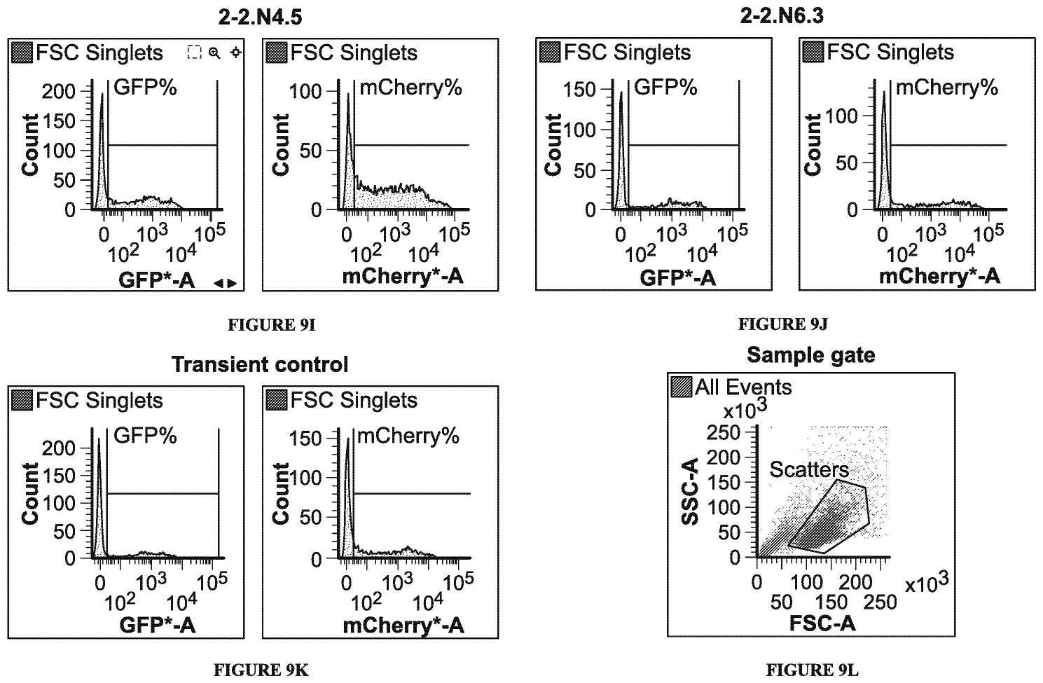

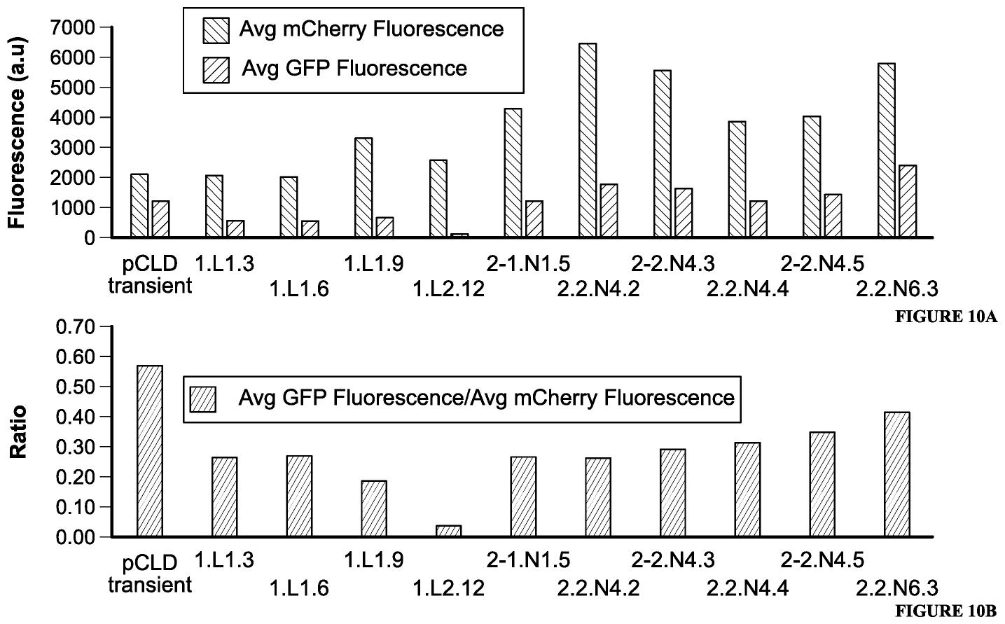

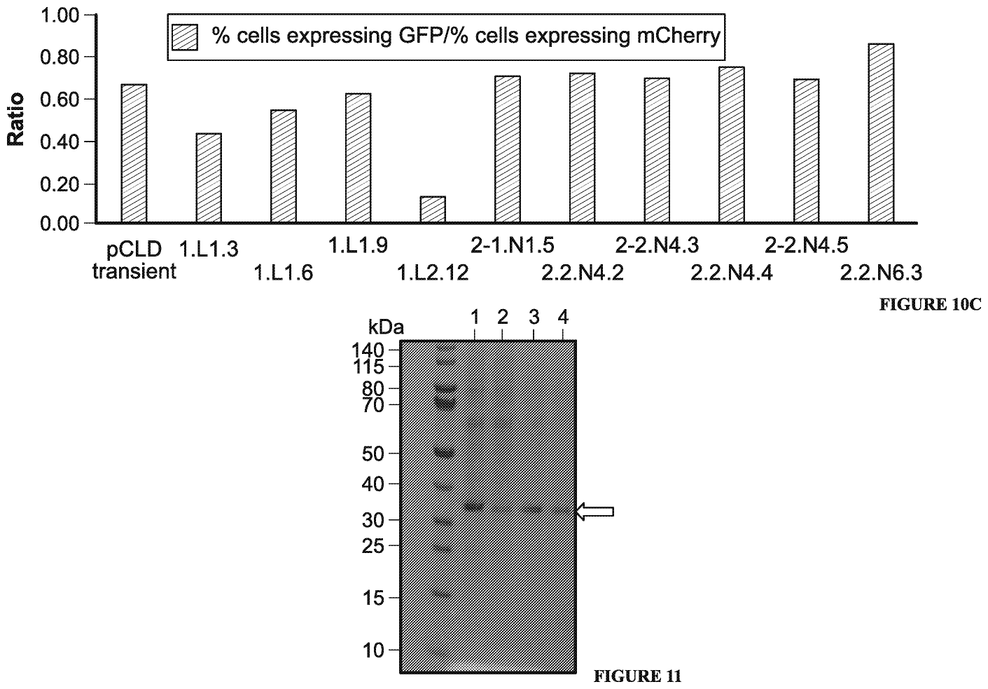

Isolated clones were then individually prepared for transfection and recharacterization via the same mCherry-GFP conditional reporter described above, which served as a representative readout of UAA incorporation (step 5 of FIG. 6A). Clonal populations were transiently transfected as described above using LCA (unless otherwise described) with the fluorescent reporter and screened by fluorescence microscopy (FIG. 8), FACS (FIG. 9). Quantification of suppression efficiency and protein production from the FACS results are shown in FIG. 10. In FIG. 8, clonal isolates were initially compared via fluorescence microscopy to parental cells expressing mCherry-GFPwt (abbreviated as MGwt) or parental cells co-transfected with mCherry-GFP* (abbreviated as MG*, * referring to a TAG mutant) and the pCLD suppressor plasmid originally used to generate the stable cell lines, referred to as “Transient control” or “pCLD transient” (plasmid 2, TABLE 2). FIG. 8A and FIG. 8B depict the standard lipofectamine based characterization assay described above (analyzed at 48 hours) for clones isolated from either lipofectamine or nucleofection based cell line generation. Representative images for clones 1.L1w.6, 2.2N4S.3, and 2.2N6S.3 demonstrated that these clones had overall higher protein expression and a higher percentage of cells showing readthrough of the MG* reporter than parental lines.

The fluorescence intensity of the transfected cells was quantified by FACS. Histogram analysis of the MG* reporter expressed in clonal populations (FIG. 9) facilitates insight into the overall percentage of cells in the population which can incorporate UAAs as well as allow for comparison of protein expression productivity and suppression efficiency. Histogram analysis of MG* expression in stable clones (FIGS. 9A-9J) relative to the transient control of CHO-dhFr containing reporter and suppressor plasmid (FIG. 9K; plasmid 2 of TABLE 2) showed the stable clones had an overall higher cell transfection efficiency, and therefore, higher reporter expression in the presence of 0.25 mM LCA, gated as shown in FIG. 9I. Clones carrying the Leu-tR-RS gene delivered by Nucleofector showed higher cell transfection efficiency, as demonstrated in FIG. 9.

The histogram depicted in FIG. 10 was determined using the BD Melody Software. Quantification of the average mCherry or GFP fluorescence, shown in FIG. 10A, depicted the same trend as seen in FIG. 9. To gain an understanding of the relative suppression efficiency, the ratio of average GFP fluorescence divided by the average mCherry fluorescence was compared across cell lines. The stable cell lines depicted in FIG. 10B displayed a reasonable suppression efficiency and demonstrated the non-trivial capability of the 4×Leu-tRNA/1×LeuRS.v1 ratio to generate stable cell lines which can incorporate LCA, without the requirement of additional suppressor plasmid DNA. Additionally, FIG. 10C depicts a higher ratio of the percentage of cells expressing GFP over the percentage of cells expressing mCherry, which confirmed a higher frequency of UAA incorporation among the stable population as compared to transient transfection. Further experimentation and analysis of the ratio of tRNA:aaRS and the site of integration may improve these characteristics.

Productivity analysis of stable clones was performed with the use of a GFP* reporter, comparing the parental cell line or clone 1.L1w.6 (FIG. 11). 2 ml of cell culture were transfected with 3 μg of reporter plasmid encoding GFP protein with an amber codon at Y39 and fused with a His tag at the C-terminal (GFP39-TAG) using Nucleofection in the presence of 0.25 mM LCA and allowed to express for 72 hours post transfection in incubator conditions as described above. Cells were harvested and lysed in 150 μL CelLytic M lysis buffer supplemented with 1× Halt protease inhibitor and 0.01% Pierce universal nuclease and affinity captured on 50 μL Ni-NTA beads following the manufacturer's protocol. Ni-NTA beads were subjected to 4 washes with PBS plus 20 mM imidazole followed by 50 μL elutions with PBS plus 300 mM imidazole. Each sample was denatured in 4X-SDS sample buffer and analyzed by Coomassie gel. Equal loading volume of 14 μL from each sample was resolved on 4-12% Bis-Tris gel in 1X MES running buffer (as shown in FIG. 11). Lane 1 contains GFPwt transfected in parental line CHO-DhFr, Lane 2 contains GFP-TAG and pCLD suppressor plasmid (plasmid 2, TABLE 2) transfected in parental line CHO-DhFr, Lane 3 contains GFPwt transfected in clone 1.L1.6, Lane 4 contains GFP-TAG without additional plasmid transfected in clone 1.L1.6 (FIG. 11). The correct size of GFP was observed and indicated by the arrow. Comparable levels of protein were shown to be expressed in the parental and clonal lines, with an apparently higher relative ratio of UAA containing protein versus wild-type (e.g., lane 3 vs 4 compared to lane 2 vs 1).

Stability of the clonal cell lines for UAA incorporation capability is further assayed via the above methods over a 30 day period. Clones are thawed and cultured according to the protocol as described above. At the end of the culture period, cells are thawed and fluorescence analysis is repeated as described above for each timepoint, followed by the general productivity analysis as described above (step 6 of FIG. 6A). For cell lines that maintain UAA incorporation capability, production lines are generated, where a gene of interest is stably integrated into the genome of the platform line (step 7 of FIG. 6A).

A summary of the constructs for construction of stable cell lines expressing leucyl suppressor tRNAs and leucyl-tRNA synthetases described in this Example is depicted in TABLE 2.

TABLE 2

| | | | | Resistance |
|---|---|---|---|---|
| # | Plasmid | # tRNAs | # aaRS | gene |
| 1 | pCLD-4xLeutRwt-LeuRS.v1-Puro | 4 | 1 | Puromycin |
| 2 | pCLD-4xLeutR.h1-LeuRS.v1-Puro | 4 | 1 | Puromycin |

### Example 3—Comparison of Stable Cell Line Pools Generated with WT Leucyl tRNA Amber Suppressor Versus H1 Leucyl tRNA Amber Suppressor

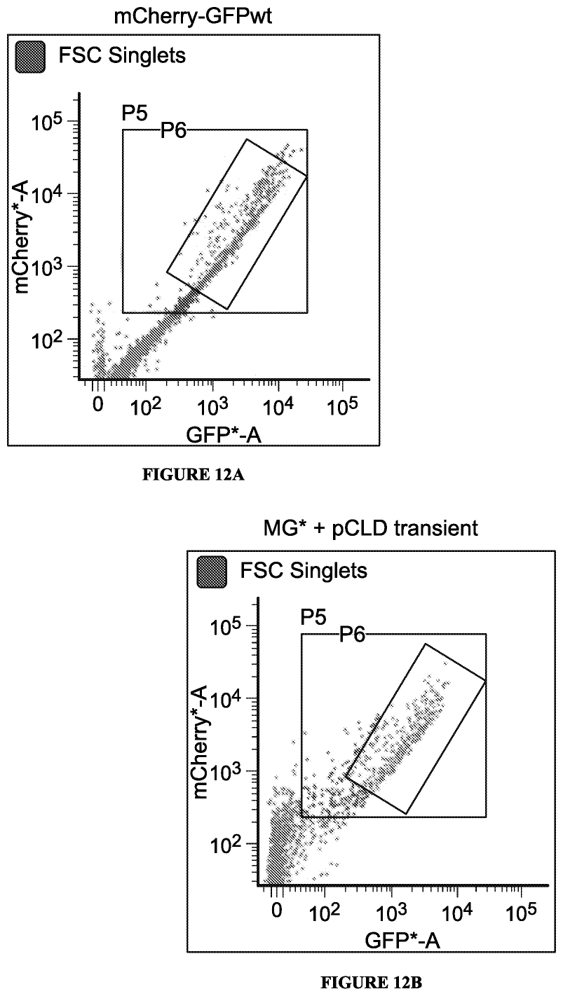

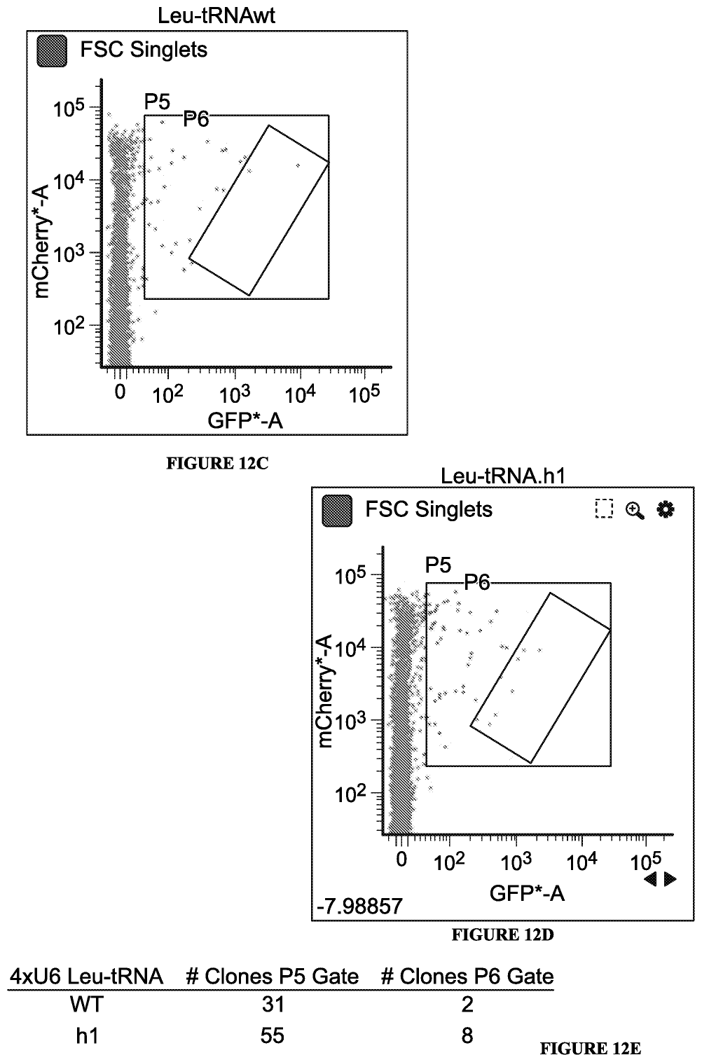

Parallel cell line pools were generated with nucleofection as described in Example 2 above using pCLD-4×LeutRwt-LeuRS.v1-Puro (plasmid 1 of TABLE 2) or pCLD-4×LeutR.h1-LeuRS.v1-Puro (plasmid 1 of TABLE 2), with wildtype or mutein tRNA h1 engineered to contain the CUA anticodon, in order to compare the effect of the wildtype versus h1 leucyl tRNAs, respectively, on the efficiency of stable clone generation (the process as shown in steps 1-3 of FIG. 6A and FIG. 6B). The leucyl suppressor tRNA LeutRwt in plasmid 1 is depicted in SEQ ID NO: 16, the leucyl suppressor tRNA LeutR.h1 in plasmid 2 is depicted in SEQ ID NO: 19, and the LeuRS.v1 leucyl-tRNA synthetase in plasmids 1 and 2 is depicted in SEQ ID NO: 2. Both pools were subjected to the same selection conditions and analyzed via FACS analysis using an MG* reporter as described in Example 2 above. A transient transfection using a pCLD plasmid expressing the h1 tRNA was used as a control to identify the target gate, P6. Approximately 2× more clones were identified within the 45 degree gate (P6) and 2× more positive clones were identified (P5) when cells were transfected with the h1 tRNA rather than the “wild-type” suppressor (FIG. 12).

### Example 4—Construction of Stable Cell Lines Expressing Tryptophanyl tRNAs and Tryptophanyl tRNA-Synthetase Muteins

Cell line pools were generated by nucleofection as described above in Example 2 using pCLD-4×TrptR-TGA-TrpRS.h14-Puro. This version of the pCLD plasmid contains (i) a 4×U6 promoter, Trp-tRNA-UCA repeat cassette and (ii) an EF1α promoted TrpRS.h14-IRES-puromycin cassette. The tryptophanyl suppressor tRNA Trp-tRNA-UCA in plasmid 1 is depicted in SEQ ID NO: 50, and the TrpRS.h14 tryptophanyl-tRNA synthetase in plasmid 1 is depicted in SEQ ID NO: 44.

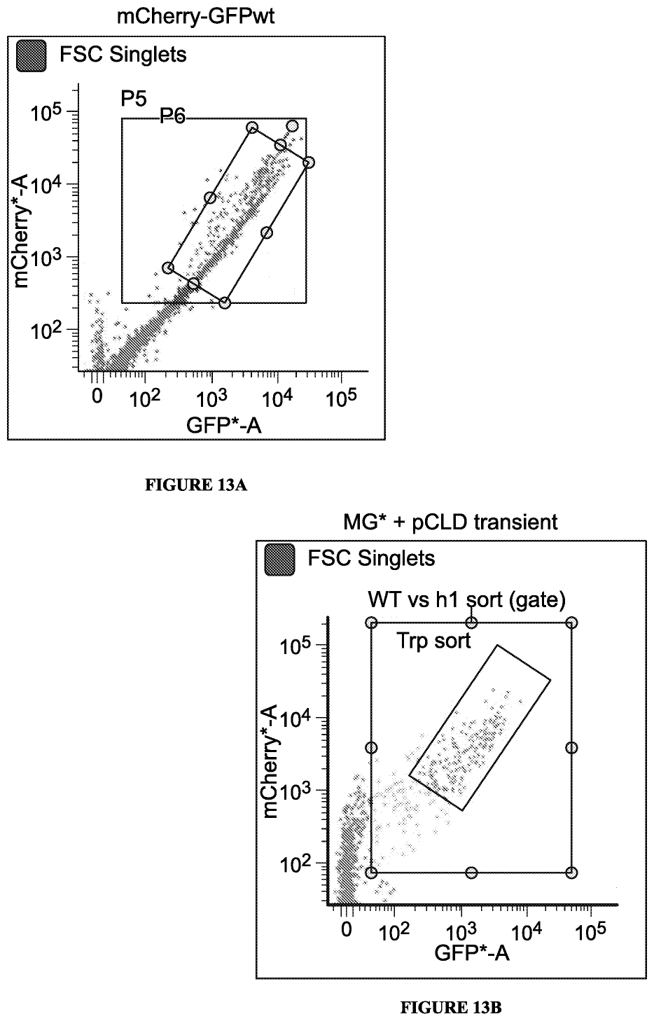

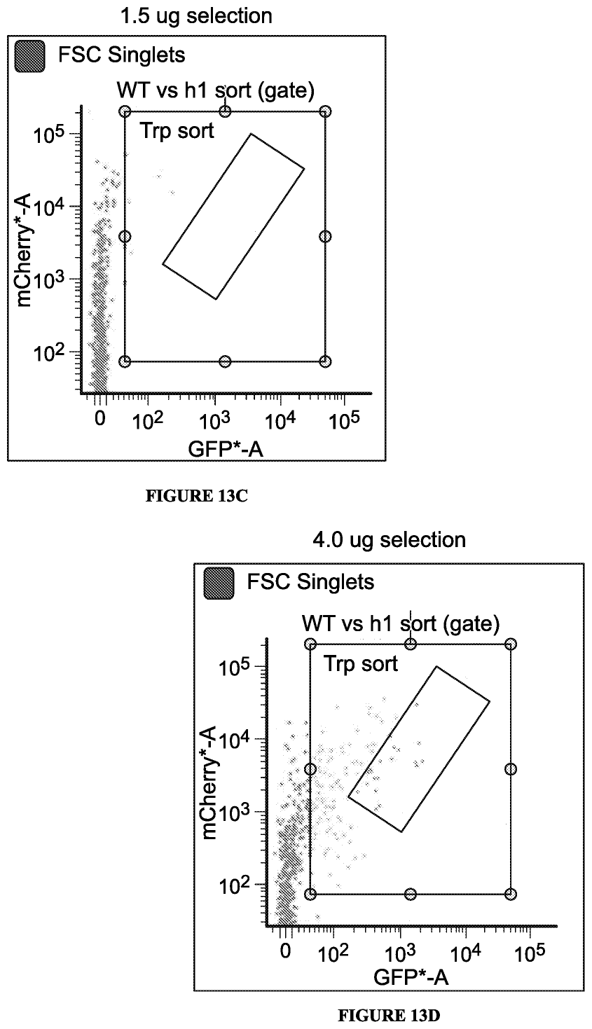

A shortened puromycin selection of 7-10 days, in either 1.5 μg/mL or 4 μg/mL, was used, and an mCherry-GFP* reporter was used, with a TGA stop codon rather than a TAG stop codon. The TGA stop codon displays higher efficiency than the TAG stop codon in mammalian cells for tryptophanyl pairs. Pools of stable tryptophan cell lines were subjected to the same selection conditions and analyzed via FACS analysis using the modified MG* (TGA) reporter as described above in Example 2 in the presence of 1 mM 5-hydroxytryptophan, HTP, the UAA for the tryptophanyl pair (FIG. 13). Stable clones were identified within the target 45 degree gate and sorted into clonal isolates. The identification and sorting of these stable clones confirms the 4:1 tRNA:aaRS ratio as viable for the generation of stable tryptophanyl cell lines.

Further characterization of clonal isolates, for example, as was performed with the leucyl clonal isolates described in Example 2 above, is conducted to determine protein production and stability characteristics of the stable tryptophanyl cell lines.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A prokaryotic leucyl tRNA synthetase mutein capable of charging a tRNA with an unnatural amino acid for incorporation into a protein, the tRNA synthetase mutein comprising the amino acid sequence of SEQ ID NO: 1 and (i) at least one substitution (e.g., a substitution with a hydrophobic amino acid) at a position corresponding to His537, (ii) at least one amino acid substitution selected from E20V, E20M, L41V, L41A, Y499H, Y499A, Y527I, Y527V, Y527G, and any combination thereof, (iii) at least one amino acid substitution selected from E2OK and L41S and any combination thereof and at least one amino acid substitution selected from M40I, T252A, Y499I, and Y527A, and any combination thereof, or (iv) a combination of two or more of (i), (ii) and (iii).

2. The tRNA synthetase mutein of claim 1, wherein the tRNA synthetase mutein comprises E20K, M40I, L41S, T252A, Y499I, Y527A, or H537G, or a combination thereof.

3. The tRNA synthetase mutein of claim 2, wherein the tRNA synthetase mutein comprises E20K, M40I, L41S, T252A, Y499I, Y527A, and H537G.

4. The tRNA synthetase mutein of any one of claims 1-3, wherein the tRNA synthetase mutein comprises a substitution at position 20 with an amino acid other than a Glu or Lys.

5. The tRNA synthetase mutein of claim 4, wherein the substitution is with a hydrophobic amino acid (e.g., Leu, Val, or Met).

6. The tRNA synthetase mutein of claim 5, wherein the tRNA synthetase mutein comprises E20M, M40I, L41S, T252A, Y499I, Y527A, and H537G.

7. The tRNA synthetase mutein of claim 5, wherein the tRNA synthetase mutein comprises E20V, M40I, L41S, T252A, Y499I, Y527A, and H537G.

8. The tRNA synthetase mutein of any one of claims 1-3, wherein the tRNA synthetase mutein comprises a substitution at position 41 with an amino acid other than a Leu or Ser.

9. The tRNA synthetase mutein of claim 8, wherein the substitution is with a hydrophobic amino acid other than Leu (e.g., Gly, Ala, Val, or Met).

10. The tRNA synthetase mutein of claim 9, wherein the tRNA synthetase mutein comprises E20K, M40I, L41V, T252A, Y499I, Y527A, and H537G.

11. The tRNA synthetase mutein of claim 9, wherein the tRNA synthetase mutein comprises E20K, M40I, L41A, T252A, Y499I, Y527A, and H537G.

12. The tRNA synthetase mutein of any one of claims 1-3, wherein the tRNA synthetase mutein comprises a substitution at position 499 with an amino acid other than Tyr, Ile, or Ser.

13. The tRNA synthetase mutein of claim 12, wherein the substitution is with a small hydrophobic amino acid (e.g., Gly, Ala, or Val).

14. The tRNA synthetase mutein of claim 13, wherein the tRNA synthetase mutein comprises E20K, M40I, L41S, T252A, Y499A, Y527A, and H537G.

15. The tRNA synthetase mutein of claim 12, wherein the substitution is with a positively charged amino acid (e.g., Lys, Arg, or His).

16. The tRNA synthetase mutein of claim 15, wherein the tRNA synthetase mutein comprises E20K, M40I, L41S, T252A, Y499H, Y527A, and H537G.

17. The tRNA synthetase mutein of any one of claims 1-3, wherein the tRNA synthetase mutein comprises a substitution at position 527 with a hydrophobic amino acid other than Ala or Leu (e.g., Gly, Ile, Met, or Val).

18. The tRNA synthetase mutein of claim 17, wherein the tRNA synthetase mutein comprises E20K, M40I, L41S, T252A, Y499I, Y527I, and H537G.

19. The tRNA synthetase mutein of claim 17, wherein the tRNA synthetase mutein comprises E20K, M40I, L41S, T252A, Y499I, Y527V and H537G.

20. The tRNA synthetase mutein of claim 17, wherein the tRNA synthetase mutein comprises E20K, M40I, L41S, T252A, Y499I, Y527G and H537G.

21. A nucleic acid encoding the tRNA synthetase mutein of any one of claims 1-20.

22. A transfer vector comprising the nucleic acid of claim 21.

23. The transfer vector of claim 22, wherein the vector is capable of introducing the nucleic acid into a cell, whereupon the nucleic acid can integrate into the genome of the cell.

24. An engineered cell comprising the tRNA synthetase mutein of any one of claims 1-20.

25. An engineered cell comprising the nucleic acid of claim 21.

26. An engineered cell comprising the transfer vector of claim 22 or 23.

27. The cell of claim 25, wherein nucleic acid is stably integrated into the genome of the cell.

28. The cell of claim 27, wherein the nucleic acid is capable of being expressed in the cell to produce a corresponding tRNA synthetase mutein.

29. The cell of any one of claims 24-28, further comprising a suppressor leucyl-tRNA capable of incorporating an unnatural amino acid into a protein undergoing expression in the cell.

30. The cell of claim 29, wherein the suppressor leucyl-tRNA is selected from any one of SEQ ID NOs: 16-42.

31. The cell of claim 29 or 30, wherein a nucleic acid encoding the suppressor leucyl-tRNA is stably integrated into the genome of the cell.

32. The cell of claim 31, wherein the nucleic acid is capable of being expressed in the cell to produce a corresponding suppressor tRNA.

33. The cell of any one of claims 24-32, wherein the unnatural amino acid is a leucine analog.

34. The cell of claim 33, wherein the leucine analog is selected from linear alkyl halides and linear aliphatic chains comprising an alkyne, azide, cyclopropene, alkene, ketone, aldehyde, diazirine, or tetrazine functional group.

35. The cell of any one of claims 24-34, wherein the protein is expressed from a nucleic acid sequence comprising a premature stop codon.

36. The cell of claim 35, wherein the tRNA synthetase mutein is capable of charging a suppressor leucyl tRNA with an unnatural amino acid which is incorporated into the protein at a position corresponding to the premature stop codon.

37. The cell of claim 35, wherein the suppressor tRNA comprises an anticodon sequence that hybridizes to the premature stop codon and permits the unnatural amino to be incorporated into the protein at the position corresponding to the premature stop codon.

38. The cell of any one of claims 24-37, wherein the protein expressed in the cell is an antibody (or a fragment thereof), bispecific antibody, nanobody, affibody, viral protein, chemokine, antigen, blood coagulation factor, hormone, growth factor, enzyme, or any other polypeptide or protein.

39. The cell of any one of claims 24-41, wherein the cell is a prokaryotic cell (e.g., a bacterial cell).

40. The cell of any one of claims 24-41, wherein the cell is a eukaryotic cell (e.g., a mammalian cell).

41. A method of expressing a protein containing an unnatural amino acid, the method comprising culturing or growing a cell of any one of claims 24-43 under conditions that permit incorporation of the unnatural amino acid into the protein being expressed in the cell.

42. The method of claim 44, wherein the protein is expressed for at least 5 days.

43. The method of claim 44, wherein the protein is expressed for at least 10 days.

44. The method of claim 45, wherein the protein is expressed for at least 15, 20, 25, 30, or 35 days.
